(19) United States
(12) Patent Application Publication
(10) Pub. No.: US 2016/0002718 A1
(43) Pub. Date: Jan. 7, 2016

(54) SYSTEM AND METHOD FOR GENERATING OR ANALYZING A BIOLOGICAL SAMPLE

(71) Applicant: ILLUMINA, INC., San Diego, CA (US)

(72) Inventors: Dale Buermann, San Diego, CA (US); Sebastian Bohm, San Diego, CA (US)

(73) Assignee: IIIumina, Inc., San Diego, CA (US)

(21) Appl. No.: 14/771,026

(22) PCT Filed: Mar. 15, 2013
(86) PCT No.: PCT/US2013/032309

§ 371 Date: Aug. 27, 2015

(51) Int. Cl. C12Q 1/68; B01L 3/00; G01N 35/10

(52) U.S. Cl. CPC C12Q 1/6869; G01N 35/1097; B01L 3/502738; B01L 3/502715; B01L 2300/0803; B01L 2300/0864; B01L 2300/0877; B01L 2400/0487; B01L 2400/0644; B01L 2400/065

(57) ABSTRACT

A system including a reaction valve having a mating side and a valve inlet. The reaction valve includes a sample chamber, and the valve inlet is in fluid communication with the sample chamber. The system also has a manifold having an engagement surface. The manifold may include first and second manifold ports at the engagement surface. The engagement surface and the mating side of the reaction valve may be positioned adjacent to each other along an interface. The system may also include a positioning assembly that is operatively coupled to at least one of the reaction valve or the manifold. The positioning assembly is configured to move at least one of the reaction valve or the manifold along the interface to fluidly disconnect the valve inlet from the first manifold port and to fluidly connect the valve inlet to the second manifold port.

### BACKGROUND

The subject matter herein relates generally to systems for analyzing or generating biological or chemical substances and, more specifically, to systems having fluidic devices for conducting designated reactions therein.

Various protocols used for biological or chemical research include the execution of a large number of controlled reactions. The reactions may be carried out in accordance with a predetermined protocol by automated systems that have, for example, suitable fluidics, optics, and electronics. The systems may be used, for example, to generate a biological or chemical product for subsequent use or to analyze a sample to detect certain properties/characteristics of the sample. When analyzing a sample in some cases, a chemical moiety that includes an identifiable label (e.g., fluorescent label) may be delivered to a chamber where the sample is located and selectively bind to another chemical moiety of the sample. These chemical reactions may be observed or confirmed by exciting the labels with radiation and detecting light emissions from the labels. Such light emissions may also be provided through other means, such as chemiluminescence.

Some known systems use a fluidic device, such as a flowcell, that includes a flow channel (e.g., interior chamber) defined by one or more interior surfaces of the flowcell. The reactions may be carried out along the interior surfaces. The flowcell is typically positioned proximate to an optical assembly that includes a device for imaging the sample within the flow channel. The optical assembly may include an objective lens and/or a solid state imaging device (e.g., CCD or CMOS). In some cases, an objective lens is not used and the solid state imaging device is positioned immediately adjacent to the flowcell for imaging the flow channel.

Before imaging the flow channel, it may be necessary to conduct a number of reactions with the sample. For example, in one sequencing-by-synthesis (SBS) technique, one or more surfaces of the flow channel have arrays of nucleic acid clusters (e.g., clonal amplicons) that are formed through bridge PCR. After generating the clusters, the nucleic acids are “linearized” to provide single stranded DNA (sstDNA). To complete a cycle of sequencing, a number of reaction components are flowed into the flow channel according to a predetermined schedule. For example, each sequencing cycle includes flowing one or more nucleotides (e.g., A, T, G, C) into the flow channel for extending the sstDNA by a single base. A reversible terminator attached to the nucleotides may ensure that only a single nucleotide is incorporated by the sstDNA per cycle. Each nucleotide has a unique fluorescent label that emits a color when excited (e.g., red, green, blue, and the like) that is used to detect the corresponding nucleotide. With the newly-incorporated nucleotides, an image of numerous clusters is taken in four channels (i.e., one for each fluorescent label). After imaging, another reaction component is flowed into the flow channel to chemically cleave the fluorescent label and the reversible terminator from the sstDNA. The sstDNA is then ready for another cycle. Accordingly, a number of different reaction components are provided to the flow channel for each cycle. A single sequencing session may include numerous cycles, such as 100, 300, or more.

The fluids that include the reaction components are typically held in a storage device (e.g., tray or cartridge) in which different fluids are stored in different reservoirs. Due to the number of reaction components and the large number of cycles, however, the total volume of fluid that is used during one session can be quite large. In fact, for some applications, it is impractical to supply the total volume of reaction components in a single cartridge. For such applications, it may be necessary to use a larger system, multiple systems, or to execute numerous sessions with a single system. These solutions can be costly, inconvenient, or unreasonable in some circumstances.

In addition to the above, the reaction components may be delivered through fluid lines that extend from the storage device to the flowcell. During operation of the system, a reaction component flows from one of the reservoirs in the storage device through the corresponding fluid line to the flowcell. The volume within the fluid line alone can be substantial. For example, the volume within a portion of a single fluid line that extends from the storage device to the flowcell may be greater than the entire volume of the flow channel. At times, however, the volume within this portion of the fluid line is not necessary because the reactions have already occurred. This volume may be referred to as “swept volume” or “dead volume.” When the system utilizes a number of cycles, as described above, the total swept volume that accumulates is not an insignificant amount and can significantly increase the cost and/or space required for operating the system.

### BRIEF DESCRIPTION

Embodiments described herein may include various systems, methods, assemblies, devices, apparatuses, and the like that can be used in connection with generating a substance and/or analyzing a substance. In particular embodiments, the substance is a biological or chemical substance.

For example, in one embodiment, a system is provided that includes a reaction valve having a mating side and a valve inlet. The reaction valve includes a sample chamber, and the valve inlet is in fluid communication with the sample chamber. The system also has a manifold having an engagement surface. The manifold may include first and second manifold ports at the engagement surface. The engagement surface and the mating side of the reaction valve may be positioned adjacent to each other along an interface. The valve inlet and the first and second manifold ports open to the interface. The valve inlet may be configured to separately align with the first and second manifold ports. The system may also include a positioning assembly that is operatively coupled to at least one of the reaction valve or the manifold. The positioning assembly is configured to move at least one of the reaction valve or the manifold along the interface to fluidly disconnect the valve inlet from the first manifold port and to fluidly connect the valve inlet to the second manifold port.

In some aspects, the reaction valve includes a cell stage and, optionally, a flowcell that is coupled in a fixed position to the cell stage. The cell stage may include the valve inlet. The flowcell may be a discrete component that is coupled (e.g., affixed or removably coupled) to the cell stage. The flowcell may include an optically transparent layer. The sample chamber may be at least partially defined by the transparent layer.

The reaction valve may include a valve passage that fluidly couples the valve inlet and the sample chamber. The valve passage may have a swept volume that is at most 20 μl. In particular configurations, the valve passage may be at most 10 μl or, more particularly, at most 6 μl.

In certain embodiments, at least one of the reaction valve or the manifold is rotatable about an axis of rotation by the positioning assembly. The positioning assembly may rotate either or both of the reaction valve and the manifold to fluidly disconnect the valve inlet from the first manifold port and to fluidly connect the valve inlet to the second manifold port. The reaction valve may also include a valve outlet that is in fluid communication with the valve inlet through the sample chamber. In some cases, the axis of rotation can extend through the valve outlet.

The sample chamber may include a flow channel that extends along a channel flow path. The flow path may extend along (e.g., may coincide with or extend parallel to) an imaging plane. In certain embodiments, the flow path includes an arcuate portion. In more particular embodiments, the arcuate portion has a radius of curvature in which a center of the radius of curvature extends through the aforementioned axis of rotation.

The valve outlet may be in fluid communication with the valve inlet through the sample chamber. Like the valve inlet, the valve outlet may open to the interface. The manifold may include a waste port that opens to the interface and is in fluid communication with the valve outlet.

In some embodiments, at least one of the reaction valve or the manifold is movable by the positioning assembly in a linear direction. For example, either or both of the reaction valve and the manifold can slide along the interface to fluidly disconnect the valve inlet from the first manifold port and to fluidly connect the valve inlet to the second manifold port.

In certain embodiments, the system also includes a detector assembly that is configured to obtain data relating to a sample within the sample chamber. Examples of such data include images and/or measurements of certain properties of the sample in the sample chamber, such as electrical properties. For such embodiments in which the detector assembly includes an imaging assembly, the imaging assembly may be positioned proximate to the sample chamber and configured to image the sample chamber.

The imaging assembly can include an objective lens having an optical axis. In some cases, the reaction valve may be movable by the positioning assembly in a direction that is transverse to the optical axis. The objective lens may be located proximate to the reaction valve and have a fixed position as the reaction valve is moved transverse to the optical axis. The positioning assembly may move the reaction valve in a common direction to (a) fluidly disconnect the valve inlet from the first manifold port and fluidly connect the valve inlet to the second manifold port and to (b) position the sample chamber relative to the objective lens for imaging the sample chamber. The sample chamber may be positioned for imaging when the valve inlet is fluidly connected to the second manifold port. In particular embodiments, each of (a) and (b) are accomplished with a single motive stroke.

The imaging assembly may be configured to obtain an image of a first portion of the sample chamber when the valve inlet is in fluid communication with the first manifold port and may be configured to obtain an image of a second portion of the sample chamber when the valve inlet is fluidly disconnected from the first manifold port. In some cases, the imaging assembly may include first and second objective lenses that are positioned with respect to the sample chamber. The first and second objective lenses may be configured to image different portions of the sample chamber.

In certain embodiments, the manifold may include a third manifold port. The positioning assembly may be configured to move and align the valve inlet with the first, second, and third manifold ports a plurality of times in accordance with a predetermined protocol. The predetermined protocol may be a sequencing-by-synthesis (SBS) protocol for sequencing nucleic acids.

In another embodiment, a method of providing fluids to a reaction valve that includes a sample chamber is provided. The reaction valve may be positioned adjacent to a manifold along an interface between the reaction valve and the manifold. The method includes moving the manifold and the reaction valve relative to each other along the interface. The reaction valve has a valve inlet fluidly connected to the sample chamber, and the manifold has first and second manifold ports. The moving operation includes moving the valve inlet along the interface to fluidly disconnect the valve inlet with the first manifold port and to fluidly connect the valve inlet with the second manifold port. The method also includes flowing a first fluid through the second manifold port and into the sample chamber of the reaction valve and moving the manifold and the reaction valve relative to each other to fluidly disconnect the valve inlet with the second manifold port and fluidly connect the valve inlet with one of the first manifold port or a third manifold port. The method may also include flowing a second fluid through said one of the first manifold port or the third manifold port and into the sample chamber of the reaction valve.

At least one of the moving operations may include rotating at least one of the reaction valve or the manifold about an axis of rotation. The sample chamber may include a flow channel that extends parallel to an imaging plane during each of the moving operations. In some embodiments, at least one of the moving operations may include a single motive stroke that fluidly connects the valve inlet to the corresponding port and positions the sample chamber for imaging. In some cases, at least one of the moving operations includes moving the reaction valve in a linear direction.

In particular embodiments, an objective lens having an optical axis that intersects the sample chamber is positioned proximate to the sample chamber. The objective lens may, but need not, be moved along a plane that is orthogonal to the optical axis between obtaining the first image and the second image.

In another embodiment, a system is provided that includes a reaction valve having a sample chamber and a positioning assembly that is operatively coupled to the reaction valve and configured to rotate the reaction valve about an axis of rotation. The system also includes an imaging assembly that is configured to obtain at least one image of the sample chamber. The positioning assembly is configured to rotate the reaction valve to move the sample chamber relative to the imaging assembly. The positioning assembly may be configured to at least one of (a) rotate the reaction valve after the imaging assembly has imaged the sample chamber or (b) rotate the reaction valve as the imaging assembly images the sample chamber.

The imaging assembly may have an imaging plane. The sample chamber moves along the imaging plane as the reaction valve is rotated. In some embodiments, the at least one image includes first, second, and third images and the imaging assembly is configured to sequentially obtain the first, second, and third images. The positioning assembly may be configured to rotate the reaction valve after the first image is obtained to position the sample chamber for the second image and also configured to rotate the reaction valve after the second image is obtained to position the sample chamber for the third image. The images may be acquired during rotational motion that is, for example, a step-and-shoot motion, or a continuous scanning motion.

In some cases, the at least one image may include first and second images. The reaction valve may be in the first rotational position when the first image is obtained and in a different rotational position when the second image is obtained. The at least one image may include a wide-field image.

In another embodiment, a method of imaging a sample in a reaction valve is provided. The method includes providing a reaction valve having a sample chamber and an imaging assembly configured to image the sample chamber. The method also includes rotating the reaction valve about an axis of rotation to a first rotational position. The sample chamber moves relative to the imaging assembly. The method also includes imaging the sample chamber and rotating the reaction valve about the axis of rotation to a second rotational position.

The imaging assembly may include an objective lens for imaging the sample chamber. In particular embodiments, the objective lens may be fixed in a lateral direction along the imaging plane when the sample chamber is moved. If necessary, the reaction valve may be moved by a positioning assembly. In some embodiments, more than one objective lens may be used and the imaging operation includes imaging multiple portions of the sample chamber concurrently.

In yet another embodiment, a fluid-selector assembly is provided that includes a cell stage configured to have a flowcell coupled thereto. The cell stage includes a valve passage that is configured to be in fluid communication with a sample chamber of the flowcell when the flowcell is coupled thereto. The cell stage has a mating side and a valve inlet that opens to the mating side. The valve inlet is in fluid communication with the valve passage. The fluid-selector assembly may also include a manifold having an engagement surface that includes first and second manifold ports. The engagement surface and the mating side of the cell stage are positioned adjacent to each other along an interface. The cell stage and the manifold may be slidably engaged such that at least one of the cell stage and the manifold may be moved to fluidly disconnect the valve inlet from the first manifold port and to fluidly connect the valve inlet to the second manifold port.

In some embodiments, the fluid-selector assembly may include a positioning assembly that is operatively coupled to at least one of the cell stage or the manifold. The positioning assembly may be configured to move at least one of the cell stage or the manifold to fluidly disconnect the valve inlet from the first manifold port and to fluidly connect the valve inlet to the second manifold port.

The cell stage may include a cell-receiving surface that is configured with a flowcell coupled thereto. In some embodiments, the cell-receiving surface defines a chamber recess that is in fluid communication with the valve inlet. The sample chamber may be formed when the flowcell is coupled to the cell-receiving surface. The sample chamber may be in fluid communication with the valve inlet.

In yet another embodiment, a reaction valve is provided that includes a cell stage having a cell-receiving surface and first and second passages that extend through the cell stage and have respective ports located at the cell-receiving surface. The reaction valve also includes a flowcell having a sample chamber (e.g., flow channel). The flowcell is coupled to the cell-receiving surface and positioned thereon such that the ports of the first and second passages are in fluid communication through the sample chamber.

The flow channel may have a non-linear channel flow path along a sample region. The flow path may have a uniform radius of curvature along the sample region. The cell stage and the flowcell may be affixed to each other such that the cell stage and the flowcell constitute a single unitary body.

In yet another embodiment, a reaction valve is provided that includes a cell stage having a cell-receiving surface and first and second passages that extend through the cell stage and have respective ports located at the cell-receiving surface. The cell-receiving surface defines a channel recess. The reaction valve may also include a flowcell coupled to the cell-receiving surface over the channel recess thereby defining a flow channel of the reaction valve. The ports of the first and second passages are in fluid communication through the flow channel.

The flow channel may have a non-linear channel flow path along a sample region, wherein the flow path has a uniform radius of curvature along the sample region. In some embodiments, the cell stage and the flowcell are affixed to each other such that cell stage and the flowcell constitute a single unitary body.

In another embodiment, a method of at least one of generating or analyzing a biological or chemical sample using a reaction valve is provided. The reaction valve includes a sample chamber and is positioned adjacent to a manifold along an interface between the reaction valve and the manifold. The reaction valve has a valve inlet, and the manifold has a plurality of ports. The valve inlet and the plurality of ports open to the interface. The method includes: (a) flowing, through a first port of the manifold, a first fluid into the sample chamber, the first fluid including a reaction component; (b) moving the manifold and the reaction valve relative to each other along the interface to fluidly disconnect the valve inlet from the first port and to fluidly connect the valve inlet with the second port; (c) flowing a second fluid through the second port and into the sample chamber; and (d) imaging at least a portion of the sample chamber.

The method may include repeating (a)-(d) a plurality of times. The reaction component may include at least one labeled nucleotide configured to react with the sample. The method may also include flowing a third fluid into the sample chamber after (d) to remove a component of the sample. Flowing a third fluid into the sample chamber may include cleaving the component of the sample, wherein the component is a reversible terminator. Element (d) of the method may include imaging a first portion of the sample chamber before or during step (b), and imaging a second portion of the sample chamber during or after step (b).

In some embodiments, the sample includes nucleic acids and the method includes performing sequencing-by-synthesis (SBS) to obtain sequencing information about the sample.

It is understood that the various embodiments described herein are intended to be illustrative, and not restrictive. For example, the embodiments described herein (and/or aspects thereof) may be used in combination with each other. In addition, many modifications may be made to adapt a particular situation or material to the teachings herein.

### BRIEF DESCRIPTION OF THE DRAWINGS

FIG. 1 is a diagram of a system for performing biological or chemical analysis in accordance with one embodiment.

FIG. 2 is an exploded view of a fluid-selector assembly formed in accordance with one embodiment that may be used with the system of FIG. 1.

FIG. 3 is a perspective view of a cross-section of the fluid-selector assembly of FIG. 2.

FIG. 4 is a perspective view of a portion of the fluid-selector assembly and an imaging assembly formed in accordance with one embodiment.

FIG. 5 is a plan view of a reaction valve in accordance with one embodiment at two different rotational positions. The reaction valve may be used with the fluid-selector assembly of FIG. 2 and the system of FIG. 1.

FIG. 6 is a plan view of another reaction valve in accordance with one embodiment at two different rotational positions and also illustrates two objective lenses for imaging the reaction valve.

FIG. 7 includes plan views of a number of different reaction valves that are each formed in accordance with one embodiment. The reaction valves may be used with the fluid-selector assembly of FIG. 2 and the system of FIG. 1.

FIG. 8 includes plan view of different reaction valves that are each formed in accordance with one embodiment and may be used with the system of FIG. 1

FIG. 9 is side cross-section of a portion of a fluid-selector assembly formed in accordance with one embodiment that may be used with the system of FIG. 1.

FIG. 10 is side cross-section of a portion of another fluid-selector assembly formed in accordance with one embodiment that may be used with the system of FIG. 1.

FIG. 11 is the side cross-section of the fluid-selector assembly in FIG. 9 illustrating a removably coupled reaction valve.

FIG. 12 is the side cross-section of the fluid-selector assembly in FIG. 10 illustrating a removably coupled flow cell.

FIG. 13 is a perspective view of a portion of a fluid-selector assembly formed in accordance with one embodiment and illustrates fluid lines that are coupled to the fluid-selector assembly.

FIG. 14 is a perspective view of another fluid-selector assembly formed in accordance with one embodiment.

FIGS. 15A and 15B illustrate a series of rotation stages of a reaction valve formed in accordance with one embodiment during a designated assay protocol.

FIG. 16 is a plan view of a reaction valve formed in accordance with one embodiment having a plurality of flow channels or “spokes” that extend radially between a center and a periphery of the reaction valve.

FIG. 17 is a side cross-section of a fluid-selector assembly formed in accordance with one embodiment including the reaction valve of FIG. 16.

FIG. 18 is a plan view of a reaction valve formed in accordance with another embodiment having a plurality of flow channels or “spokes.”

FIG. 19 includes plan views of a fluid-selector assembly formed in accordance with another embodiment in which a reaction valve is moved in a linear direction.

FIG. 20 includes plan views of a fluid-selector assembly formed in accordance with another embodiment in which a manifold is moved in a linear direction.

FIG. 21 is a schematic view of an imaging assembly formed in accordance with one embodiment.

FIG. 22 is a perspective view of an imaging assembly formed in accordance with one embodiment.

FIG. 23 is a perspective view of an imaging assembly formed in accordance with another embodiment.

FIG. 24 is a perspective view of an imaging assembly formed in accordance with another embodiment.

FIG. 25 is a perspective view of an imaging assembly formed in accordance with another embodiment.

FIG. 26 is a schematic view of a system configured for biological or chemical analysis formed in accordance with one embodiment.

FIG. 27 is a flowchart of a method in accordance with one embodiment.

FIG. 28 is a perspective view of a fluid-selector assembly in accordance with one embodiment.

FIG. 29 is an end view of the fluid-selector assembly of FIG. 28.

FIG. 30 is another end view of the fluid-selector assembly of FIG. 28.

FIG. 31 is a perspective view of a fluid-selector assembly in accordance with one embodiment.

FIG. 32 is a perspective view of a fluid-selector assembly in accordance with one embodiment.

### DETAILED DESCRIPTION

Embodiments set forth herein may include various systems, methods, assemblies, devices, apparatuses, and the like that are used in connection with generating a substance and/or analyzing a substance. The substance may be a sample and, in particular embodiments, a biological or chemical sample. The generation or analysis of the substance may be carried out by flowing one or more reaction components into a chamber of a reaction valve that includes the substance. The reaction components are delivered through fluid lines that are in flow communication with the reaction valve. The reaction components may be provided to the sample chamber (or reaction chamber) according to a predetermined schedule or protocol. As such, reactions may be conducted within the sample chamber for generating a substance and/or analyzing a substance.

In particular embodiments, this disclosure provides a sample chamber that is (transiently or permanently) combined with a valve, thereby forming a reaction valve. The resulting physical connection and alignment between the valve and sample chamber in this integrated apparatus can be advantageous and useful. For example, the sample chamber can be fluidly coupled to several reservoirs or other fluidic devices and the sample chamber can also be observable by a detection device. The reaction valve can be moved according to a schedule that accommodates fluidic manipulations and detection of the sample. For example, movement can allow the valve to access alternative fluid lines, thereby causing sequential flow of desired fluids (e.g. reagents, wash solutions used in a nucleic acid sequencing reaction), while analytes attached to the surface of the sample chamber (e.g. nucleic acid clusters to be sequenced) pass into a detection zone. Thus, an advantage of the reaction valve is the provision of efficient motion and a relatively small number of moving parts for an analytical process that utilizes multiple fluidic steps and multiple detection steps.

Other advantages are provided as well. For example, some embodiments may be configured to reduce an amount of swept or dead volume within the fluid lines as compared to other known systems that utilize fluidic devices. Alternatively or additionally, embodiments may reduce the complexity of some optical assemblies and/or a likelihood of misalignment occurring within the optical assemblies. Embodiments set forth herein may also provide systems that require less space than known systems.

Various embodiments may enable the execution of numerous reactions in the sample chamber. Some reactions can be preparative in nature, and detection of reaction products in the chamber is not necessary. Other reactions may be analytical in nature and products can be detected or the reaction otherwise monitored in the chamber. The reactions may be detected as the reactions occur, between steps of multi-step reactions or after the reactions occur. For some applications, the reaction components (e.g., reagents, enzymes, buffer solution, and the like) are delivered in separate cycles in which each cycle includes flowing a number of fluids in a predetermined order into the sample chamber. During a single session, particular embodiments may repeat each cycle numerous times (e.g., 100, 300 or more cycles) over an extended period (e.g., hours or days). One particularly useful application may be nucleic acid sequencing through, for example, sequencing by synthesis (SBS). Some embodiments may execute predetermined protocols, whereas others may be more adaptive. For example, some protocols may be changed or reconfigured during a session based on data gathered in the session.

To this end, some embodiments may include a detector assembly that is positioned relative to the sample chamber or that is operatively coupled to the sample chamber so that a characteristic or quality of the substance can be detected. By way of example, the detector assembly may be an imaging assembly that is configured to detect optical signals from the sample chamber. In such cases, the sample chamber may be at least partially defined by an optically transparent surface that permits the optical signals to propagate therethrough. Alternatively or additionally, the detector assembly may be configured to determine other properties or characteristics of the substance (e.g., electrical properties through ion concentration). For those embodiments that detect optical signals, movement of an objective lens or a solid-state detector that is proximate to the sample chamber is not necessary and may be limited. More specifically, an objective lens may have a fixed position or may only be permitted to move along the z-axis, which is unlike many other known systems in which the objective lens must be moved along a lateral plane that is orthogonal to the z-axis.

At least some embodiments may benefit from a reaction valve that includes a stage and a flowcell that is coupled to the stage. In addition to supporting the flowcell, the stage may also operate as part of a valve system. More specifically, the stage may be movably mounted to a manifold having a number of ports. The stage may include one or more passages that are fluidly connected to one or more of the ports based on a position of the stage relative to the manifold. In particular embodiments, the stage may be moved to fluidly disconnect the flowcell from one port and fluidly connect the flowcell to another port while also repositioning the flowcell for imaging. In some embodiments, the system may be configured so that a predetermined area of the flowcell is positioned for imaging when a the flowcell is in a predetermined position relative to the ports and/or when a predetermined fluid is, or has been, provided to the flowcell.

Such embodiments may be particularly useful for applications in which numerous cycles (e.g. each cycle including a sequence of two or more steps) are repeated. For example, embodiments set forth herein can be used to obtain images of nucleic acid features that are present in nucleic acid arrays, such as those used in nucleic acid sequencing applications. A variety of nucleic acid sequencing techniques that utilize optically detectable samples and/or reagents can be used. These techniques may be particularly well suited to the embodiments described herein and therefore may highlight various advantages for particular embodiments. Although nucleic acid sequencing applications are exemplified, such advantages can be extended to other biological or chemical applications. Examples of other biological or chemical applications include, but are not limited to, nucleic acid synthesis, protein synthesis, protein sequencing, polymer synthesis, combinatorial library synthesis, cell-based assays, enzyme assays and the like. Alternative embodiments may also be suitable for applications that are not biological or chemical. Examples of other applications include but are not limited to environmental screening, detection of encoded particles, and the like.

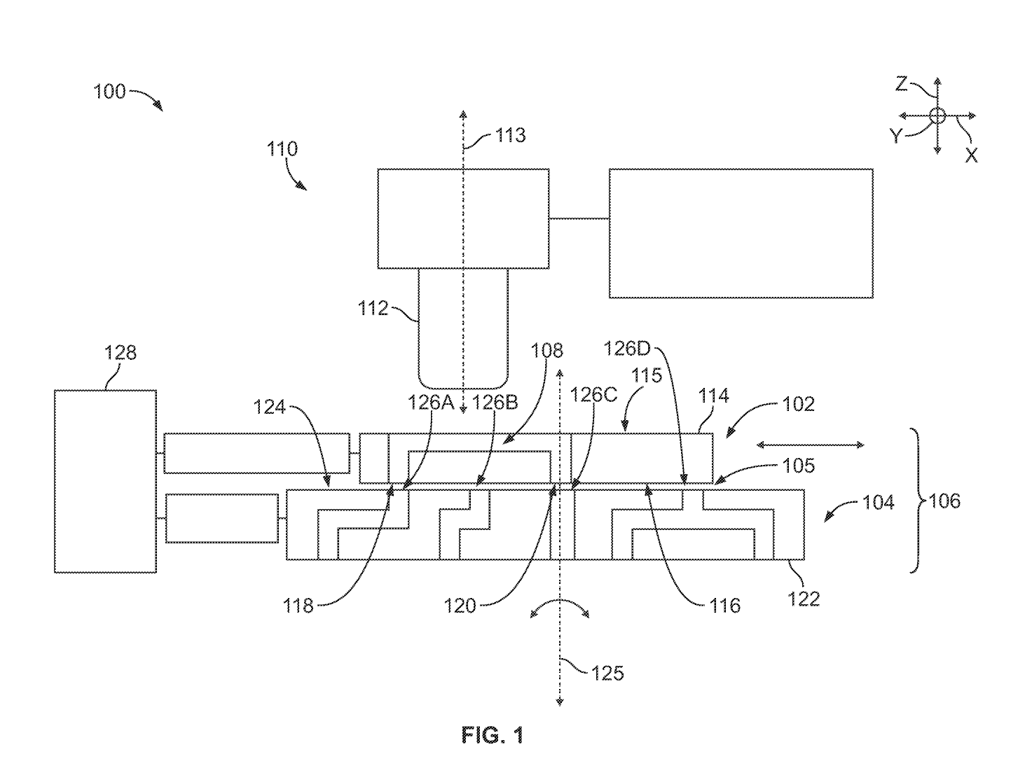

FIG. 1 is a block diagram of a system 100 formed in accordance with one embodiment. The system 100 is particularly configured for biological and/or chemical analysis of a sample, but the system 100 may have other applications apparent from the exemplary components and methods set forth herein. As shown in FIG. 1, the system 100 is oriented with respect to mutually perpendicular x-, y-, and z-axes. The system 100 may include a reaction valve 102 and a manifold 104 that are operatively engaged to each other with an interface 105 therebetween. The reaction valve 102 and the manifold 104 may be referenced collectively as a fluid-selector assembly 106. In some cases, the fluid-selector assembly 106 also includes a positioning assembly 128 as described below.

The fluid-selector assembly 106 is configured to selectively transfer designated fluids between the reaction valve 102 and the manifold 104. For example, the manifold 104 may include a number of passages (e.g., conduits, fluid lines, etc.) that are in fluid communication with one or more reservoirs (not shown) holding chemical components. Different fluids containing reaction components may be delivered through the manifold 104 and across the interface 105 into the reaction valve 102. In FIG. 1, the reaction valve 102 and the manifold 104 appear to have a gap therebetween along the interface 105. However, this is shown to more clearly illustrate the various components. The interface 105 may be a sealed interface to impede or prevent inadvertent leakage of fluid outside of a designated pathway. Optionally, a gasket layer (not shown) may extend along all or part of the interface 105.

The reaction valve 102 is configured to hold a substance (not shown) within a sample or reaction chamber 108, which may be referred to as a flow channel for some embodiments. The substance may be a biological or chemical sample, such as nucleic acids. During operation of the system 100, the reaction valve 102 and the manifold 104 may be moved relative to each other to selectively connect the sample chamber 108 to different passages of the manifold 104. Moving the reaction valve 102 and the manifold 104 with respect to each other to selectively connect the sample chamber 108 to different passages may also be described as adjusting the fluid-selector assembly 106. The relative movement may entail movement of the valve 102 relative to a stationary manifold 104, moving the manifold 104 relative to a stationary valve 102, or movement of both the valve 102 and the manifold 104.

Optionally, the sample chamber 108 may be located proximate to and/or operatively coupled to a detector assembly 110. In the illustrated embodiment, the detector assembly 110 is an imaging assembly that includes at least one objective lens 112. The objective lens 112 has an optical or imaging axis 113 that extends longitudinally through a center of the objective lens 112 and intersects the reaction valve 102. The optical axis 113 extends parallel to the z-axis. In some embodiments, the detector assembly 110 is configured to move the objective lens 112 in a z-direction to move the objective lens 112 closer to or further from the reaction valve 102. In some embodiments, the detector assembly 110 may also move the objective lens along the lateral plane defined by the x- and y-axes.

The detector assembly 110 may also include an imaging assembly that has one or more solid-state imaging devices (e.g., CCD or CMOS sensor) (not shown) positioned immediately adjacent to the reaction valve 102. For instance, a solid-state imaging device may be pressed against the reaction valve 102. In some cases, the solid-state imaging device can receive the optical signals without the use of optics for focusing the optical signals. However, other detector assemblies may use optics (e.g., objective lens 112, a collection of microlenses, etc.) to direct the optical signals toward the solid-state imaging device or, more specifically, designated pixels of the solid-state imaging device.

In some embodiments, a detector assembly may include one or more detectors (e.g., electrodes, sensors, or transducers) that are proximate to or within the sample chamber and are configured to detect a property or characteristic of the substance. Non-limiting examples of such properties or characteristics include optical characteristics of the substance (e.g. fluorescence, luminescence, chemiluminescence, electrochemical luminescence, or absorbance), electrical properties of the substance (e.g., voltage, current, conductivity, impedance), thermal properties of the substance, a mass of the substance, or a pressure of the sample chamber. Many of these detectors may be manufactured using techniques associated with micro-electro-mechanical systems (MEMS). In such embodiments, the detector assembly 110 may be integrated as part of the reaction valve 102. For instance, a flowcell may be mounted to or formed with a solid-state device that includes microfabricated detectors. As evident from these examples, the detector assembly 110 can detect optical signals, but is not required to detect optical signals. For embodiments that detect optical signals, an excitation radiation source is optional. For example, chemiluminescence reactions can be detected without the need for an excitation radiation source. However, an excitation radiation source can be useful for some modes of detection, such as fluorescence or luminescence. An excitation source can be located external or internal to the reaction valve and can be configured to illuminate a sample therein. Particularly useful biosensors are described in greater detail in international application no. PCT/US2011/057111 (published as WO 2012/058096), filed on Oct. 20, 2011, which is incorporated herein by reference in its entirety. As another example, the detector assembly 110 may include a microcircuit arrangement as described in U.S. Pat. No. 7,595,883, which is incorporated herein by reference in the entirety.

Yet in some embodiments, the system 100 may not include a detector assembly. In such cases, the system 100 may be exclusively dedicated to generating a substance in which it may not be necessary to monitor the substance. The substance may then be subsequently used in another application. By way of example only, the system 100 may be used to generate a field of primers immobilized to a surface in the sample chamber or to synthesize chemical molecules.

As shown in FIG. 1, the reaction valve 102 includes a device body 114 that has mounting and mating sides 115, 116, which may also be referred to as first and second outer surfaces, respectively. In the illustrated embodiment, the mounting and mating sides 115, 116 are sides of the device body 114 that face in opposite directions such that a thickness of the device body 114 extends therebetween. As shown, the mating side 116 is positioned adjacent to the manifold 104. The device body 114 may include a valve inlet 118 and a valve outlet 120 that are in fluid communication with each other through the sample chamber 108. In the illustrated embodiment, the valve inlet 118 and the valve outlet 120 open to the mating side 116 and the interface 105. However, in other embodiments, only one of the valve inlet 118 or the valve outlet 120 opens to the mating side 116, and the other may open to a different surface, such as the mounting side 115.

The manifold 104 includes a base substrate 122 and has an engagement surface 124. The base substrate 122 may include an array of ports 126, such as the ports 126A-126D in FIG. 1. Each of the ports 126A-126D may open to the engagement surface 124 and the interface 105. As shown, the mating side 116 of the reaction valve 102 and the engagement surface 124 of the manifold 104 are positioned adjacent to each other along the interface 105. The interface 105 may be characterized as a slidable interface in that embodiments may include sliding the reaction valve 102 and the manifold 104 alongside each other. In some cases, the mating side 116 and the engagement surface 124 have designated characteristics or properties that facilitate forming a sealed interface so that fluid is prevented (or significantly impeded) from leaking along the interface 105. For example, the mating side 116 and the engagement surface 124 may be hydrophobic, when the fluid that is transferred to the sample chamber is a hydrophilic liquid, or may be hydrophilic when the fluid that is transferred is a non-polar liquid (e.g., oil).

The system 100 may also include a positioning assembly 128 that is operatively coupled to at least one of the reaction valve 102 or the manifold 104. The positioning assembly 128 may be configured to move the reaction valve 102 and the manifold 104 with respect to each other or, in other words, adjust the fluid-selector assembly 106. To this end, the positioning assembly 128 may include one or more actuators, structural elements, and/or motors that operatively engage the reaction valve 102 and/or the manifold 104 in a designated manner. For example, the positioning assembly 128 may be configured to move the reaction valve 102 and/or the manifold 104 to fluidly connect the valve inlet 118 with the first manifold port 126A and then move the reaction valve 102 and/or the manifold 104 to fluidly connect the valve inlet 118 with another port, such as the port 126B.

In particular configurations, the reaction valve 102 and/or the manifold 104 are rotated about an axis of rotation 125. The axis of rotation 125 may extend parallel to the optical axis 113. For example, the reaction valve 102 may be rotated about the axis of rotation 125 to move the valve inlet 118 of the reaction valve 102 relative to the ports 126. The axis of rotation 125 may extend through the port 126C and the valve outlet 120 as shown in FIG. 1. The port 126C and the valve outlet 120 may remain fluidly connected with each other when the reaction valve 102 is rotated about the axis of rotation 125. In such embodiments, the port 126C may be a waste port that receives fluid from the sample chamber 108.

In other embodiments, the reaction valve 102 and/or the manifold 104 may be moved in one or more linear directions. For example, the positioning assembly 128 may move the reaction valve 102 in a linear direction along a lateral plane defined by the x- and y-axes to fluidly connect the valve inlet 118 with one of the ports 126 and/or to fluidly connect the valve outlet 120 with one of the ports 126.

When the positioning assembly 128 repositions the reaction valve 102 relative to the manifold 104, the fluid within a particular port 126 that is not in fluid communication with the sample chamber 108 may be primed. For example, the port 126A may be sealed (e.g., blocked) by the mating side 116 when the reaction valve 102 and the manifold 104 are moved with respect to each other. As such, when the valve inlet 118 returns and fluidly connects with the port 126A, the fluid is ready (e.g., primed) to immediately flow into the reaction valve 102. Due to the proximity of the sample chamber 108 to the port 126A, the swept or dead volume may be significantly reduced or substantially eliminated relative to other known systems.

In accordance with a designated protocol, the positioning assembly 128 may adjust the fluid-selector assembly 106 so that fluids may be provided to the sample chamber 108 in a particular order for carrying out designated reactions. The designated protocol may include a number of coordinated actions that are executed by the system 100 for completing a biological application. The actions may include, for example, movements of the reaction valve 102 and/or the manifold 104, pumping certain fluids through the system 100 in a predetermined order or sequence, detecting a characteristic of the sample (e.g., imaging the sample), and, optionally, analyzing data obtained about the sample. For instance, the reaction components for SBS sequencing may be delivered to the sample chamber 108 in an order as described herein. Optical signals from the sample chamber 108 may be detected by the detector assembly 110 and the data obtained may be analyzed to gather information about a sample.

In some embodiments, various factors are configured so that the detector assembly 110 may image the sample chamber 108 (or detect a certain property) without moving along the lateral plane defined by the x- and y-axes. In particular embodiments, the factors may be configured so that the sample chamber 108 may be fluidly connected to designated ports 126 while the images are obtained or while the property is detected. Such factors may include a spatial location of the detector assembly 110, locations of the ports 126 along the engagement surface 124, dimensions and shape of the sample chamber 108, the fluids that are to be delivered through the ports 126, and/or an order that different fluids are to be delivered to the sample chamber 108. Other factors may also be considered.

Accordingly, when the sample chamber 108 is fluidly connected to the port 126A, the detector assembly 110 may image a portion of the sample chamber 108. The fluid-selector assembly 106 may then be adjusted by the positioning assembly 128 such that (a) the reaction valve 102 has a different spatial relationship with respect to the detector assembly 110, whereby a different portion of the sample chamber 108 is imaged by the detector assembly 110, and (b) the sample chamber 108 is not in fluid communication with the port 126A, optionally, being in fluid communication with a new port instead. Fluid may be directed into the sample chamber 108 through the new port, and the detector assembly 110 may image another portion of the sample chamber 108. In this manner, the sample chamber 108 may have designated fluids delivered into the sample chamber 108 in a predetermined order while the detector assembly 110 obtains information about the designated reactions occurring within the sample chamber 108.

In alternative embodiments, the direction of flow may be in the opposite direction described above. More specifically, the direction of flow in an alternative embodiment may be from the device outlet 120 to the device inlet 118. In such cases, the reaction valve 102 may be used to deliver designated volumes of fluid to one or more of the ports 126. For example, the reaction valve 102 may be used to aliquot a predetermined volume of fluid in the sample chamber 108 with one or more of the ports 126. In this alternative embodiment, the ports 126A and 126B may provide access to chambers or wells. After a designated reaction is carried out within the sample chamber 108, the device inlet 118 may be fluidly connected to one of the ports 126 and a predetermined volume of the substance in the sample chamber 108 may flow through the device inlet 118 and into the port 126. In this alternative embodiment, the port 126C may deliver the reaction components to the sample chamber 108.

As used herein, a “sample chamber” or a “reaction chamber” is a space where a fluid can be contained during a synthetic or analytical process. The process can be, for example, a designated biological or chemical reaction. Exemplary reactions are synthesis reactions, binding reactions, degradation reactions and sequencing reactions. The space is defined by one or more surfaces and, in some cases, the designated reactions may occur immediately along the surface(s), such as when oligonucleotides or sstDNA are immobilized to one or more of the surface(s). In some cases, the sample chamber is defined by surfaces. For example, the sample chamber may be defined between top and bottom surfaces in which each surface is planar and extends parallel to the other. The planar surfaces may substantially coincide with an imaging plane of an objective lens. In other cases, however, one or more surfaces of the sample chamber may not be planar. For instance, the sample chamber may include an array of recesses or wells where the designated reactions occur. Furthermore, the term sample chamber is not limited to a single continuous space, but may include multiple spaces that are separated from each other (e.g., by walls).

In many cases, the surfaces of the flowcell will be modified to facilitate conducting or performing the designated reactions within the sample chamber. For instance, an interior or exterior surface of the flow cell can be chemically changed and/or physically changed. In physical modification, a surface may be roughened, pitted, patterned, shaped, etched, or smoothed to facilitate holding a substance within the sample chamber. For example, a surface may be physically modified to facilitate immobilizing desired biomolecules thereon or to facilitate deterring immobilization of unwanted biomolecules. The exterior and/or interior surfaces of the flow cell may also be roughened, smoothed, pitted, patterned, etched, or shaped to produce desired effects on light transmission through the flow cell. For example, exterior and/or interior surfaces may be shaped to increase radiation of light energy onto predetermined portions of the sample chamber. A surface of the sample chamber may have induced modulations in the index of refraction of the material, ridges or grooves (e.g., gratings) formed thereon to increase an intensity of radiation on biomolecules attached to the interior surface.

In chemical modification, a surface that defines the sample chamber may be chemically modified to couple to a designated chemical moiety (e.g., biomolecule, linker, etc.) that facilitates carrying out the assay. For example, a surface of the sample chamber can have a moiety that acts as a chemical linker or precursor to a chemical linker. Any of a variety of linker moieties and precursor moieties known in the art can be used, examples of which include, but are not limited to, those described in U.S. Patent Publication No. 2006/0057729 A1, and U.S. Pat. No. 7,504,499, each of which is incorporated herein by reference in its entirety. A surface of the sample chamber can be chemically modified to incorporate a linker precursor or linker moiety using methods known to those skilled in the art or readily ascertainable based on the properties of the surface, linkage chemistry, and substance to be linked to the surface. In particular embodiments, the surfaces that define the sample chamber are planar surfaces that include primers immobilized thereto. The planar surface may have multiple planar areas in which each of the planar areas is captured entirely within a single image by an imaging assembly. The multiple planar areas may form one continuous planar area.

Chemical modification may also include selectively immobilizing desired biomolecules to at least one of the interior and exterior surfaces. As used herein, the term “immobilized,” when used with respect to a biomolecule, includes substantially attaching the biomolecule at a molecular level to a surface. For example, biomolecules may be immobilized to a surface of the microbody using adsorption techniques including non-covalent interactions (e.g., electrostatic forces, van der Waals, and dehydration of hydrophobic interfaces) and covalent binding techniques where functional groups or linkers facilitate attaching the biomolecules to the surface. Immobilizing biomolecules to a surface of a microbody may be based upon the properties of the microbody surface, the liquid medium carrying the biomolecules, and the properties of the biomolecules themselves. In some cases, a surface may be first modified to have functional groups bound to the surface. The functional groups may then bind to biomolecules to immobilize the biomolecules to the surface. In some embodiments, biomolecules that are immobilized to one or more surfaces that define the sample chamber include labels that emit optical signals.

Nucleic acids can be immobilized to the surface using a solid phase amplification technique. For example, a nucleic acid can be attached to a surface and amplified using bridge amplification. Useful bridge amplification methods are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420. Another useful method for amplifying nucleic acids on a surface is rolling circle amplification (RCA), for example, using methods set forth in further detail below.

The sample chamber may be configured such that the designated reactions are detectable within the sample chamber. For example, the sample chamber may be partially defined by a transparent layer of material that permits optical signals to propagate therethrough. As another example, the space of the sample chamber may be located along sensors, actuators, or detectors so that one or more properties of the reactions may be detected.

For those cases in which the fluid flows into and exits the sample chamber, the sample chamber may be part of a flow channel. A flow channel fluidly connects an incoming (or upstream) passage that provides fluid and an outgoing (or downstream) passage through which the fluid exits. The flow channel may have different dimensions than the incoming and outgoing passages. For example, cross-sections taken transverse to the direction of flow for the incoming passage and the flow channel may have different heights or widths. However, in other cases, the cross-sections have the same or nearly identical dimensions. In one or more embodiments, the flow channel has a single inlet and a single outlet. However, other embodiments may include more than one inlet and/or more than one outlet.

A sample chamber or flow channel may have any of a variety of shapes. For example, the sample chamber may have a channel flow path that has a curved shape. The shape can include a single curve or a number of curves. In other cases, the sample chamber may be a linear channel that extends along a straight line between the inlet and the outlet, for example, having chambers that run in a direction akin to the direction of spokes in a wheel.

As used herein, the terms “inlet” and “outlet” may be substituted with the term “port.” Many of the ports described herein open to an interface between two components. For example, a cell stage may be movably mounted to a manifold along an interface. Each of the cell stage and the manifold may have one or more ports that open to the interface. When the cell stage and/or the manifold is moved, one or more ports along the cell stage may be aligned with one or more ports along the manifold. Some ports may open to a sample chamber.

As used herein, two features of a channel or fluid network are “in fluid communication” with each other if at least a portion of the fluid (e.g., liquid or gas) flowing through one of the features in a designated direction is configured to be transferred to the other feature. A feature may be a structurally defined feature, such as a port (e.g., inlet or outlet), a chamber or channel, a designated portion of a chamber or channel, a fluid line, and the like. To provide specific examples, the valve inlet 118 and the valve outlet 120 in FIG. 1 are in fluid communication with each other through the sample chamber 108. As shown in FIG. 1, the valve inlet 118 may be in fluid communication with the port 126A along the interface 105. As such, the valve inlet 118 is also in fluid communication with the fluid line associated with the port 126A. However, the valve inlet 118 is not in fluid communication with the port 126B along the interface 105 as shown in FIG. 1 since the positions of the reaction valve 102 and the manifold 104 do not permit fluid to flow therebetween.

In some cases, a channel may branch or divide into multiple channels or a plurality of channels may converge into a single channel. As such, a single feature prior to the channel diverging can be in fluid communication with multiple features that are not in fluid communication with each other based on the direction of flow. Likewise, in the case of separate channels converging, multiple features that are not in fluid communication with each other may be in fluid communication with a single feature after the channels converge based on the direction of flow.

As used herein, the phrase “to fluidly connect” means to bring together, align open or otherwise connect two features such that liquid or gas can flow between the two features. As used herein, the term “to fluidly disconnect” means to separate, misalign or obstruct or otherwise disconnect two features that were fluidly connected such that liquid or gas will no longer flow between the two features. As one example, when the reaction valve 102 as shown in FIG. 1 is relatively moved by the positioning assembly 128, the valve inlet 118 and the port 126A are fluidly disconnected. The valve inlet 118 may then be, for example, fluidly connected to the port 126B and its associated fluid line. If two features are in fluid communication then the two features are fluidly connected. Likewise, if two features are not in fluid communication then the two features are fluidly disconnected.

As used herein, a “designated reaction” includes or causes a change in at least one of a chemical, electrical, physical, or optical property or quality of a substance. For example, the designated reaction may be a chemical transformation, chemical change, or chemical interaction. In some embodiments, the designated reactions may be detected by an imaging assembly. The imaging assembly may include an optical assembly that directs optical signals to a sensor (e.g., CCD or CMOS). However, in other embodiments, the imaging assembly may detect the optical signals directly. For example, a flowcell may be mounted onto a solid state device (e.g., CMOS sensor). However, the designated reactions may also cause a change in electrical properties. For example, the designated reaction may be a change in ion concentration within a solution.

Exemplary reactions include, but are not limited to, chemical reactions such as reduction, oxidation, addition, elimination, rearrangement, esterification, amidation, etherification, cyclization, or substitution; binding interactions in which a first chemical binds to a second chemical; dissociation reactions in which two or more chemicals detach from each other; fluorescence; luminescence; chemiluminescence; and biological reactions, such as nucleic acid replication, nucleic acid amplification, nucleic acid hybridization, nucleic acid ligation, phosphorylation, enzymatic catalysis, receptor binding, or ligand binding. The designated reaction can also be addition or elimination of a proton, for example, detectable as a change in pH of a surrounding solution or environment.

The designated reactions may be facilitated by or in response to a stimulus. A stimulus can be at least one of physical, optical, electrical, magnetic, and chemical. For example, the stimulus may be an excitation light that excites fluorophores in a substance. The stimulus may also be a change in a surrounding environment, such as a change in concentration of certain biomolecules (e.g., enzymes or ions) in a solution. The stimulus may also be an electrical current applied to a solution within a predefined volume. In addition, the stimulus may be provided by shaking, vibrating, or moving a sample chamber where the substance is located to create a force (e.g., centripetal force). As used herein, the phrase “facilitated by or in response to a stimulus” includes more direct responses to a stimulus (e.g., when a fluorophore emits energy of a specific wavelength after absorbing incident excitation light) and more indirect responses to a stimulus in that the stimulus initiates a chain of events that eventually results in the response (e.g., incorporation of a base in pyrosequencing eventually resulting in chemiluminescence). The stimulus may be immediate (e.g., excitation light incident upon a fluorophore) or gradual (e.g., change in temperature of the surrounding environment).

Various embodiments include providing a reaction component to a sample. As used herein, a “reaction component” or “reactant” includes any substance that is capable of reacting with a sample or that may be used to carry out an assay or protocol to obtain the designated reactions. Non-limiting examples of a “reaction component” or “reactant” include one or more of reagents, samples, targets, analytes, polymerases, primers, denaturants, linearization mixes for linearizing DNA, enzymes suitable for a particular assay (e.g., cluster amplification or SBS), nucleotides, oligonucleotide primers, nucleic acids, cleavage mixes, oxidizing protectants, other biological or chemical molecules, buffer solution, and wash solution. The reaction components are typically delivered to a sample region (e.g., area where a sample is located) in a solution and/or immobilized to the sample region. The reaction components may interact directly or indirectly with the sample.

In particular embodiments, the designated reactions are detected optically through an optical assembly. The optical assembly may include an optical train of optical components that cooperate with one another to direct the optical signals to an imaging device (e.g., CCD, CMOS, or photomultiplier tubes). However, in alternative embodiments, the sample region may be positioned immediately adjacent to an activity detector (e.g., solid state device) that detects the designated reactions without the use of an optical train. The activity detector may be able to detect designated events, properties, qualities, or characteristics within a predefined volume or area. For example, an activity detector may be able to capture an image of the predefined volume or area. An activity detector may be able detect an ion concentration within a predefined volume of a solution or along a predefined area. Exemplary activity detectors include charged-coupled devices (CCD's) (e.g., CCD cameras); photomultiplier tubes (PMT's); molecular characterization devices or detectors, such as those used with nanopores; microcircuit arrangements, such as those described in U.S. Pat. No. 7,595,883, which is incorporated herein by reference in the entirety; and CMOS-fabricated sensors having field effect transistors (FET's), including chemically sensitive field effect transistors (chemFET), ion-sensitive field effect transistors (ISFET), and/or metal oxide semiconductor field effect transistors (MOSFET).

In certain embodiments, the optical assembly includes an objective lens that is located adjacent to the sample chamber. The objective lens may be configured to direct excitation radiation (e.g., from one or more lasers) onto one or more of the surfaces of the sample chamber and receive light emissions (e.g., fluorescence) from the one or more surfaces of the sample chamber. By way of example only, a working distance that exists between the surface of the sample chamber that is being imaged and the objective lens may be less than 5000 microns or, more specifically, less than 2000 microns. In particular embodiments, the working distance between the surface and the objective lens may be less than 1000 microns. However larger working distances may be used in other embodiments.

When imaging, the optical assembly may be configured for diffraction-limited focusing and imaging of only one of the surfaces that defines the sample chamber. For example, the sample chamber may be defined by first and second opposite surfaces. The first and second surfaces may be planar and extend parallel to each other along the sample chamber. During an imaging session, the optical assembly may be configured to image only the first surface or only the bottom surface. In some embodiments, the optical assembly may be configured to image one of the first and second surfaces and then image the other of the first and second surfaces. In such embodiments, a compensator may be selectively used by the optical assembly to image both of the surfaces. Optical assemblies that may be suitable for one or more embodiments are described in U.S. Patent Appl. Publ. Nos. 2013/0023422 and 2011/0220775, which are each hereby incorporated by reference in its entirety.

Certain embodiments include objective lenses having high numerical aperture (NA) values. Exemplary high NA ranges for which embodiments may be particularly useful include NA values of at least about 0.6. For example, the NA may be at least about 0.65, 0.7, 0.75, 0.8, 0.85, 0.9, 0.95, or higher. Those skilled in the art will appreciate that NA, being dependent upon the index of refraction of the medium in which the lens is working, may be higher including, for example, up to 1.0 for air, 1.33 for pure water, or higher for other media such as oils. However, other embodiments may have lower NA values than the examples listed above. Image data obtained by the optical assembly may have a resolution that is between 0.1 and 50 microns or, more particularly, between 0.1 and 10 microns. Optical assemblies may have a resolution that is sufficient to individually resolve the features or sites that are separated by a distance of less than 15 μm.

In general, the NA value of an objective lens is a measure of the breadth of angles for which the objective lens may receive light. The higher the NA value, the more light that may be collected by the objective lens for a given fixed magnification. This is because the collection efficiency and the resolution increase. As a result, multiple objects may be distinguished more readily when using objectives lenses with higher NA values because a higher feature density may be possible. Therefore, in general, a higher NA value for the objective lens may be beneficial for imaging. However, as the NA value increases, its sensitivity to focusing and imaging-through media thickness variation also increases. In other words, lower NA objectives lenses have longer depth of field and are generally not as sensitive to changes in imaging-through media thickness.

Certain embodiments may detect features on a surface at a rate of at least about 0.01 mm/sec. Depending upon the particular application, faster rates can also be used including, for example, in terms of the area scanned or otherwise detected, a rate of at least about 0.02 mm2/sec, 0.05 mm2/sec, 0.1 mm2/sec, 1 mm2/sec, 1.5 mm2/sec, 5 mm2/sec, 10 mm2/sec, 50 mm2/sec, 100 mm2/sec, or faster. If desired, for example, to reduce noise, the detection rate can have an upper limit of about 0.05 mm2/sec, 0.1 mm2/sec, 1 mm2/sec, 1.5 mm2/sec, 5 mm2/sec, 10 mm2/sec, 50 mm2/sec, or 100 mm2/sec.

As used herein, the term “optical signals” or “light signals” includes electromagnetic energy capable of being detected. The term includes light emissions from labeled biological or chemical substances and also includes transmitted light that has been refracted, reflected, or partially absorbed by the substance. Optical or light signals, including excitation radiation that is incident upon the sample and light emissions that are provided by the sample, may have one or more spectral patterns. For example, more than one type of label may be excited in an imaging session. In such cases, the different types of labels may be excited by a common excitation light source or may be excited by different excitation light sources at different times or at the same time. Each type of label may emit optical signals having a spectral pattern that is different from the spectral pattern of other labels. For example, the spectral patterns may have different emission spectra. The light emissions may be filtered to separately detect the optical signals from other emission spectra.

Different elements and components described herein may be removably coupled. As used herein, when two or more elements or components are “removably coupled” (or “removably mounted,” and other like terms) the elements are readily separable without destroying the coupled components. Elements can be readily separable when the elements may be separated from each other without undue effort, without the use of a tool (i.e. by hand instead), or without a significant amount of time spent in separating the components. By way of example, a flowcell may be removably coupled to a cell stage. As another example, a reaction valve that includes a flowcell and a cell stage may be removably coupled to a manifold. In such embodiments, the flowcell or the reaction valve may be previously prepared in some manner and then mounted to the manifold to conduct an assay protocol. For example, oligonucleotides may be immobilized to at least one of the surfaces that define the sample chamber before the flowcell and/or the reaction valve are mounted to the manifold.

It is noted that terms, such as “coupled” or “mounted,” do not define or include a particular relationship with respect to gravity. For example, a flowcell may be mounted to a cell stage without the cell stage being located below (with respect to gravity) the flowcell. Unless explicitly claimed otherwise, structural components described herein do not have a specific relationship with respect to the direction of gravity.

As used herein, when elements are “operatively coupled,” “operatively engaged,” “operably coupled” and the like, the two elements are coupled or engaged such that the elements can perform a designated function or achieve a designated result including, for example, any of a variety of the functions or results set forth herein. Being operatively or operably coupled includes the elements being directly coupled or indirectly coupled.

During a detecting session, properties of the sample are detected or obtained. With respect to imaging sessions, various types of imaging may be used with embodiments described herein. For example, embodiments described herein may utilize a “step and shoot” procedure in which different portions of a sample area are individually detected or imaged between (or after) relative movements of the detector and sample. Embodiments may also be configured to perform at least one of epi-fluorescent imaging and total-internal-reflectance-fluorescence (TIRF) imaging. Embodiments described herein may utilize a “scanning” procedure in which different portions of a sample area are detected or imaged during movement between the sample and detector. In some embodiments, the imaging assembly includes a scanning time-delay integration (TDI) system. Furthermore, the imaging sessions may include “line scanning” one or more samples such that a linear focal region of light is scanned across the sample(s). Some methods of line scanning are described, for example, in U.S. Pat. No. 7,329,860 and U.S. Pat. Pub. No. 2009/0272914, each of which is incorporated herein by reference in its entirety. Imaging sessions may also include moving a point focal region of light in a raster pattern across the sample(s). In alternative embodiments, imaging sessions may include detecting light emissions that are generated, without illumination, and based entirely on emission properties of a label within the sample (e.g., a radioactive or chemiluminescent component in the sample). In alternative embodiments, flowcells may be mounted onto an imager (e.g., CCD or CMOS) that detects the designated reactions.

As used herein, the term “sample” or “sample-of-interest” can include various materials or substances. In particular embodiments, a sample may include one or more biological or chemical substances. As used herein, the term “biological or chemical substances” may include any of a variety of biological or chemical substances that are suitable for being reacted, imaged or examined or have properties that are suitable for being detected. For example, biological or chemical substances include biomolecules, such as nucleosides, nucleic acids, polynucleotides, oligonucleotides, proteins, enzymes, polypeptides, antibodies, antigens, ligands, receptors, polysaccharides, carbohydrates, polyphosphates, nanopores, organelles, lipid layers, cells, tissues, organisms, and biologically active chemical compound(s) such as analogs or mimetics of the aforementioned species. Other chemical substances include labels that can be used for identification, examples of which include fluorescent labels and others set forth in further detail below.

With respect to optical imaging, different types of samples may be held by different optical substrates or support structures that affect incident light in different manners. In particular embodiments, samples to be detected can be attached to one or more surfaces of a substrate or support structure, such as a flowcell. Flowcells may include one or more flow channels. In flowcells, the flow channels may be separated from the surrounding environment by top and bottom layers of the flowcell. Thus, optical signals to be detected are projected from within the support structure and may transmit through multiple layers of material having different refractive indices. For example, when detecting optical signals from an interior bottom surface of a flow channel, the optical signals that are desired to be detected may propagate through a fluid having an index of refraction, through one or more layers of the flowcell having different indices of refraction, and through the ambient environment having a different index of refraction. Optical signals can also be detected from the interior top surface, whereby the optical signals are detected after propagating through the top layer of the flow cell. An optical system of the present disclosure can include an optical compensator allowing detection from the interior surfaces at both the top and bottom of the flow cell by compensating for the difference in the two propagation paths. Thus, a flowcell or other support structure used in an apparatus or method of the present disclosure can include sample components (e.g. nucleic acid features or clusters) on one or more interior surfaces of the support structure. Exemplary configurations for such support structures and optical devices for detecting samples on multiple surfaces, for example, using an optical compensator, are set forth in U.S. Pat. No. 8,039,817, which is incorporated herein by reference in its entirety.

As used herein, a “reaction valve” is a fluidic device that includes one or more sample chambers where designated reactions may occur. The reaction valve may include one or more ports that provide fluidic access to the sample chamber(s). The reaction valve is configured to be fluidly coupled to a fluidic network of a system. By way of example, a reaction valve may be a flowcell and/or lab-on-chip device. Flowcells may hold a sample along a surface for imaging by an imaging assembly. Lab-on-chip devices may hold the sample and perform additional functions, such as detecting the designated reaction using an integrated detector. Reaction valves may optionally include additional components, such as housings or imagers, that are operatively coupled to the sample chambers. In particular embodiments, the sample chambers may have interior surfaces where a sample is located, and the reaction valve can include a transparent material that permits the sample to be imaged after a designated reaction occurs.

In particular embodiments, the reaction valves have sample chambers or channels with microfluidic dimensions. In such chambers or channels having microfluidic dimensions, the surface tension and cohesive forces of the liquid flowing therethrough and the adhesive forces between the liquid and the surfaces of the channel have at least a substantial effect on the flow of the liquid. For example, a cross-sectional area (taken perpendicular to a flow direction) of a microfluidic channel may be about 10 μm² or less.

The sample region of some embodiments may include one or more microarrays. A microarray may include a population of different probe molecules that are attached to one or more substrates such that the different probe molecules can be differentiated from each other according to relative location. An array can include different probe molecules, or populations of the probe molecules, that are each located at a different addressable location on a substrate. Alternatively, a microarray can include separate optical substrates, such as beads, each bearing a different probe molecule, or different population of the probe molecules (each population being, for example, homogeneous or heterogeneous with respect to the species of probe molecule(s) present), that can be identified according to the locations of the optical substrates on a surface to which the substrates are attached or according to the locations of the substrates in a liquid. Exemplary arrays in which separate substrates are located on a surface include, without limitation, a BeadChip Array available from Illumina®, Inc. (San Diego, Calif.) or others including beads in wells such as those described in U.S. Pat. Nos. 6,266,459, 6,355,431, 6,770,441, 6,859,570, and 7,622,294; and PCT Publication No. WO 00/63437, each of which is hereby incorporated by reference. Other arrays having particles on a surface include those set forth in US 2005/0227252; WO 05/033681; and WO 04/024328, each of which is hereby incorporated by reference.

Any of a variety of microarrays known in the art can be used. A typical microarray contains sites, sometimes referred to as features, each having a population of probes. The population of probes at each site is typically homogenous having a single species of probe, but in some embodiments the populations can each be heterogeneous. Sites or features of an array are typically discrete, being separated. The probe can be a known sequence, for example produced by a synthetic procedure, or an unknown sequence, for example, deposited from a sample that is to be sequenced or otherwise analyzed. Thus, in some contexts a probe may be referred to as a “target” or vice versa. The terms are intended to be capable of use interchangeably herein unless explicitly indicated to the contrary. The separate sites can be contiguous or they can have spaces between each other. The size of the probe sites and/or spacing between the sites can vary such that arrays can be high density, medium density or lower density. High density arrays are characterized as having sites separated by less than about 15 μm. Medium density arrays have sites separated by about 15 to 30 μm, while low density arrays have sites separated by greater than 30 μm. An array useful in some embodiments can have sites that are separated by less than 100 μm, 50 μm, 10 μm, 5 μm, 1 μm, or 0.5 μm. An apparatus or method of an embodiment can be used to image an array at a resolution sufficient to distinguish sites at the above densities or density ranges.

Further examples of commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies as described, for example, in U.S. Pat. Nos. 5,324,633; 5,744,305; 5,451,683; 5,482,867; 5,491,074; 5,624,711; 5,795,716; 5,831,070; 5,856,101; 5,858,659; 5,874,219; 5,968,740; 5,974,164; 5,981,185; 5,981,956; 6,025,601; 6,033,860; 6,090,555; 6,136,269; 6,022,963; 6,083,697; 6,291,183; 6,309,831; 6,416,949; 6,428,752 and 6,482,591, each of which is hereby incorporated by reference. A spotted microarray can also be used in a method according to an embodiment of the invention. An exemplary spotted microarray is a CodeLink™ Array available from Amersham Biosciences. Another microarray that is useful is one that is manufactured using inkjet printing methods such as SurePrint™ Technology available from Agilent Technologies.

The apparatus, systems and methods set forth herein can be used to detect the presence of a particular target molecule in a sample contacted with the microarray. This can be determined, for example, based on binding of a labeled target analyte to a particular probe or due to a target-dependent modification of a particular probe to incorporate, remove, or alter a label at the probe location. Any one of several assays can be used to identify or characterize targets using a microarray as described, for example, in U.S. Patent Application Publication Nos. 2003/0108867; 2003/0108900; 2003/0170684; 2003/0207295; or 2005/0181394, each of which is hereby incorporated by reference.

Furthermore, optical systems described herein may be constructed to include various components and assemblies as described in International Publication No. WO 2007/123744, and/or to include various components and assemblies as described in International Publication No. WO 2009/042862, both of which the complete subject matter are incorporated herein by reference in their entirety. In particular embodiments, optical systems can include various components and assemblies as described in U.S. Pat. No. 7,329,860 and WO 2009/137435, of which the complete subject matter is incorporated herein by reference in their entirety. Optical systems can also include various components and assemblies as described in U.S. Patent Application Publication No. 2010/0157086 A1, of which the complete subject matter is incorporated herein by reference in its entirety.

In particular embodiments, methods, apparatus and optical systems described herein may be used for sequencing nucleic acids. For example, sequencing-by-synthesis (SBS) protocols may be particularly applicable. In SBS, a plurality of fluorescently labeled modified nucleotides are used to sequence a plurality of clusters of amplified DNA (possibly millions of clusters) present on the surface of an optical substrate (e.g., a surface that at least partially defines a channel in a flowcell). The flowcells may contain nucleic acid samples for sequencing. The samples for sequencing can take the form of single nucleic acid molecules that are separated from each other so as to be individually resolvable, amplified populations of nucleic acid molecules in the form of clusters or other features, or beads that are attached to one or more molecules of nucleic acid. Accordingly, sequencing can be carried out on an array such as those set forth herein. The nucleic acids can be prepared such that they comprise an oligonucleotide primer adjacent to an unknown target sequence. To initiate the first SBS sequencing cycle, one or more differently labeled nucleotides, and DNA polymerase, etc., can be flowed into/through the flowcell by a fluid flow subsystem (not shown). Either a single type of nucleotide can be added at a time, or the nucleotides used in the sequencing procedure can be specially designed to possess a reversible termination property, thus allowing each cycle of the sequencing reaction to occur simultaneously in the presence of several types of labeled nucleotides (e.g. A, C, T, G). The nucleotides can include detectable label moieties such as fluorophores. Where the four nucleotides are mixed together, the polymerase is able to select the correct base to incorporate and each sequence is extended by a single base. Nonincorporated nucleotides can be washed away by flowing a wash solution through the flowcell. One or more lasers may excite the nucleic acids and induce fluorescence. The fluorescence emitted from the nucleic acids is based upon the fluorophores of the incorporated base, and different fluorophores may emit different wavelengths of emission light. A deblocking reagent can be added to the flowcell to remove reversible terminator groups from the DNA strands that were extended and detected. The deblocking reagent can then be washed away by flowing a wash solution through the flowcell. The flowcell is then ready for a further cycle of sequencing starting with introduction of a labeled nucleotide as set forth above. The fluidic and detection steps can be repeated several times to complete a sequencing run. Exemplary sequencing methods are described, for example, in Bentley et al., Nature 456:53-59 (2008), WO 04/018497; U.S. Pat. No. 7,057,026; WO 91/06678; WO 07/123744; U.S. Pat. No. 7,329,492; U.S. Pat. No. 7,211,414; U.S. Pat. No. 7,315,019; U.S. Pat. No. 7,405,281, and US 2008/0108082, each of which is incorporated herein by reference.

In some embodiments, nucleic acids can be attached to a surface, such as an inner surface of a reaction chamber, and amplified prior to or during sequencing. For example, amplification can be carried out using bridge amplification to form nucleic acid clusters on a surface. Useful bridge amplification methods are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420. Another useful method for amplifying nucleic acids on a surface is rolling circle amplification (RCA), for example, as described in Lizardi et al., Nat. Genet. 19:225-232 (1998) and US 2007/0099208 A1, each of which is incorporated herein by reference. Emulsion PCR on beads can also be used, for example as described in Dressman et al., Proc. Natl. Acad. Sci. USA 100:8817-8822 (2003), WO 05/010145, or U.S. Patent Publ. Nos. 2005/0130173 or 2005/0064460, each of which is incorporated herein by reference in its entirety.

Other sequencing techniques that are applicable for use of the methods and systems set forth herein are pyrosequencing, nanopore sequencing, and sequencing by ligation. Exemplary pyrosequencing techniques and samples that are particularly useful are described in U.S. Pat. No. 6,210,891; U.S. Pat. No. 6,258,568; U.S. Pat. No. 6,274,320 and Ronaghi, Genome Research 11:3-11 (2001), each of which is incorporated herein by reference. Exemplary nanopore techniques and samples that are also useful are described in Deamer et al., Acc. Chem. Res. 35:817-825 (2002); Li et al., Nat. Mater. 2:611-615 (2003); Soni et al., Clin Chem. 53:1996-2001 (2007) Healy et al., Nanomed. 2:459-481 (2007) and Cockroft et al., J. am. Chem. Soc. 130:818-820; and U.S. Pat. No. 7,001,792, each of which is incorporated herein by reference. In particular, these methods utilize repeated steps of reagent delivery. An instrument or method set forth herein can be configured with reservoirs, valves, fluid lines and other fluidic components along with control systems for those components in order to introduce reagents and detect signals according to a desired protocol such as those set forth in the references cited above. Any of a variety of samples can be used in these systems such as substrates having beads generated by emulsion PCR, substrates having zero-mode waveguides, substrates having integrated CMOS detectors, substrates having biological nanopores in lipid bilayers, solid-state substrates having synthetic nanopores, and others known in the art. Such samples are described in the context of various sequencing techniques in the references cited above and further in US 2005/0042648; US 2005/0079510; US 2005/0130173; and WO 05/010145, each of which is incorporated herein by reference.

Exemplary labels that can be detected in accordance with various embodiments, for example, when present on or within a support structure include, but are not limited to, a chromophore; luminophore; fluorophore; optically encoded nanoparticles; particles encoded with a diffraction-grating; electrochemiluminescent label such as Ru(bpy)32+; or moiety that can be detected based on an optical characteristic. Fluorophores that may be useful include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dyes, phycoerythin, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066, each of which is hereby incorporated by reference.

Although embodiments are exemplified with regard to detection of samples that include biological or chemical substances supported by an optical substrate, it will be understood that other samples can be imaged or have properties detected by the embodiments described herein. Other exemplary samples include, but are not limited to, biological specimens such as cells or tissues, electronic chips such as those used in computer processors, encoded particles and the like. Examples of some of the applications include microscopy, satellite scanners, high-resolution reprographics, fluorescent image acquisition, analyzing and sequencing of nucleic acids, DNA sequencing, sequencing-by-synthesis, imaging of microarrays, imaging of holographically encoded microparticles and the like.

Flow of fluids through the reaction valve may be implemented by various device(s) that are capable of controlling fluid flow and, particularly, microfluidic flow. Such devices may include valves (e.g., multi-port valves, solenoid valves, bypass valves), flow sensors, and pumps. Non-limiting examples of suitable pumps include syringe pumps, fixed volume electro-mechanical pumps, peristaltic pump, piston pumps, micropumps, piezoelectric pumps, and electro-osmotic pumps.

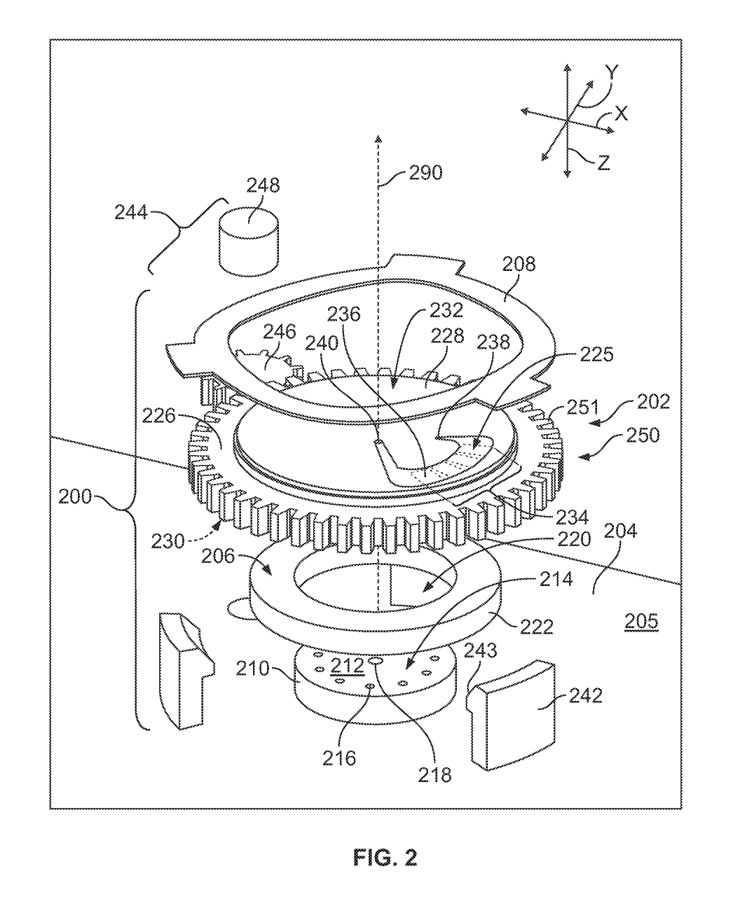

FIG. 2 is an exploded view of a fluid-selector assembly 200 that may be used for generating and/or analyzing a biological sample. The fluid-selector assembly 200 may be used as part of, and include components that are similar to, the system 100. For example, the fluid-selector assembly 200 includes a reaction valve 202, a manifold 204, a thermal element 206, and a biasing member 208. In FIG. 2, the fluid-selector assembly 200 is oriented with respect to mutually perpendicular x-, y-, and z-axes. The various components of the fluid-selector assembly 200 are configured to be stacked with respect to each other along the z-axis. In FIG. 2, the z-axis may extend parallel to the force of gravity. However, the fluid-selector assembly 200 is not required to have any particular orientation with respect to gravity.

The reaction valve 202 includes a cell stage 226 and a flowcell 228 that is coupled to the cell stage 226. The reaction valve 202 has a mating side 230 that is configured to interface with the manifold 204 and a mounting side 232 that has the flowcell 228 therealong in FIG. 2. The mating side 230 and the mounting side 232 may face in opposite directions along the z-axis. The mounting side 232 may be defined partially by a surface of the cell stage 226 and partially by a surface of the flowcell 228. As shown, the reaction valve 202 includes a sample chamber 225, which is hereinafter referred to as a flow channel 225.

As shown in FIG. 2, a channel inlet 238 and a channel outlet 240 are in fluid communication with each other through the flow channel 225. The channel inlet and outlet 238, 240 may open to the mounting side 232 and fluidly connect with the flow channel 225. A direction of flow may be from the channel inlet 238 to the channel outlet 240. However, in other embodiments, the flow may be in the opposite direction. In such cases, element 240 may be characterized as the channel inlet and element 238 may be characterized as the channel outlet.

The flow channel 225 is located proximate to or along the mounting side 232. During operation, fluid may flow into the flow channel 225 through the channel inlet 238 and exit the flow channel 225 through the channel outlet 240. In the illustrated embodiment, the flowcell 228 is optically transparent so that optical signals may be detected from at least a portion of the flow channel 225 by an imaging assembly (not shown).

The flow channel 225 includes a sample region 234. In the illustrated embodiment and others, a sample region may be a region within a flow channel or sample chamber where designated reactions are intended to occur. The sample region 234 may include an area along a surface. The surface may have, for example, biomolecules immobilized thereto that are configured to interact with reaction components that flow through the flow channel. In other embodiments, the sample region 234 may include a volume of space within the sample chamber. In some embodiments, the sample region is capable of being detected either optically or by other means. For instance, in the illustrated embodiment, the sample region 234 may be imaged. More specifically, FIG. 2 illustrates designated areas or tiles 236 that represent portions of an interior surface of the flow channel 225 that are configured to be imaged. The interior surface may be a bottom surface that defines the flow channel 225 along the sample region 234 and/or a top surface that defines the flow channel 225 along the sample region 234. In other embodiments, sensors may be located within the flow channel 225 along the sample region 234 and be configured to detect one or more properties of the substance in the sample region 234.

In certain embodiments, the sample region 234 may be a general area along the flow channel 225 where the designated reactions occur at random locations. For example, clusters of nucleic acids may be grown at random locations within a sample region. Alternatively, the sample region may be configured such that the designated reactions are more localized or occur at predetermined locations. In some cases, a surface of the flow channel may be chemically modified so that the designated reactions are more likely to occur at certain locations along the surface than others. As one example, the sample region may include a microarray having features with known locations with respect to each other. More specifically, each feature may have a distinguishable location or address that may be used to determine (e.g., through a database or table) the biomolecule of the feature.

In order to prepare the sample region 234, surfaces of the cell stage 226 and/or the flowcell 228 may be formed or chemically modified so that the designated reactions are more likely to occur at predetermined locations. For instance, surfaces of the cell stage 226 and/or the flowcell 228 may be formed with cavities or recesses (e.g., wells, channels, and the like) and/or may be chemically modified. In some cases, one or more surfaces of the cell stage 226 and/or the flowcell 228 may be patterned to have hydrophobic portions and hydrophilic portions. Alternatively or in addition to, the sample region 234 may include reaction sites that are fabricated and patterned as described in U.S. Pat. Appl. Pub. No. 2012/0316086 A1; U.S. application Ser. Nos. 13/492,661; 13/661,524; 13/783,043; and 13/787,396 and U.S. Provisional Application No. 61/625,051, filed on Apr. 16, 2012, each of which is incorporated herein by reference in its entirety.

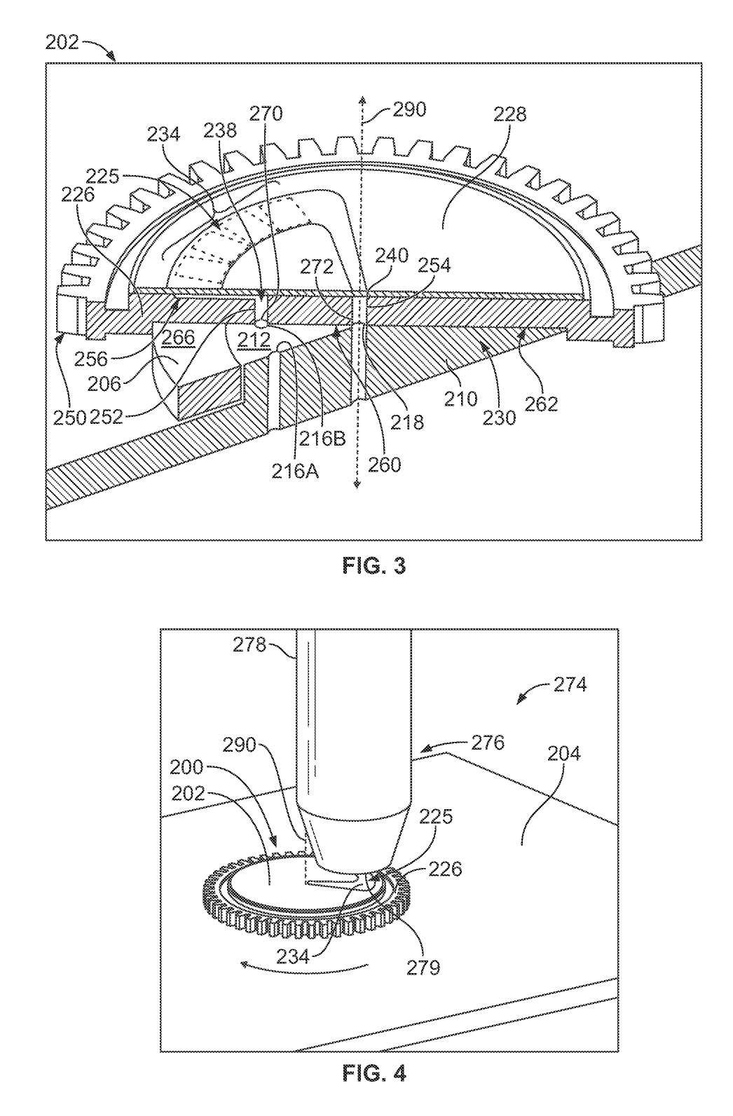

The manifold 204 has a main surface 205 and includes a base substrate or platform 210 that projects from the main surface 205. The base substrate 210 has an engagement surface 212 that extends along a xy-plane and orthogonal to the z-axis and faces the reaction valve 202. The base substrate 210 may include an array of manifold ports 214 that open to the engagement surface 212 in a direction along the z-axis. The manifold ports 214 may be distributed along the engagement surface 212 in a designated pattern that is configured with respect to a valve inlet 270 (FIG. 3) and/or a valve outlet 272 (FIG. 3) of the reaction valve 202. For example, the manifold ports 214 may include a plurality of upstream or fluid ports 216 and a waste port 218. In the illustrated embodiment, the waste port 218 is equidistant from each of the fluid ports 216, although alternative embodiments may have different spatial relationships.

The base substrate 210 is configured to function as an intermediary that provides the manifold ports 214 for transferring fluid between the reaction valve 202 and the manifold 204. In some embodiments, the base substrate 210 may also function as a platform that facilitates stable movement of the reaction valve 202. In the illustrated embodiment, each of the engagement surface 212 and a surface of the mating side 230 that interfaces with the engagement surface 212 may be dimensioned so that, when the surface of the mating side 230 and the engagement surface 212 are moved along the interface between each other, the flow channel 225 does not shift or move along the z-axis. More specifically, if the reaction valve 202 is rotated about an axis of rotation 290 that extends through the waste port 218 and the channel outlet 240, the flow channel 225 moves within an imaging plane that extends orthogonal to an optical axis (not shown) of an objective lens.

In the illustrated embodiment, the base substrate 210 has a cylindrical shape that projects from the main surface 205. The waste port 218 may be located at a geometric center of the engagement surface 212 or at a geometric center of the fluid ports 216. However, other shapes may be used for the base substrate 210. The base substrate 210 has a width or, more specifically, a diameter that is measured parallel to the xy-plane and a height that is measured along the z-axis. The base substrate 210 is sized such that the base substrate 210 may be inserted into an opening 220 of the thermal element 206. The opening 220 is defined by a thermal body 222 of the thermal element 206. The engagement surface 212 is configured to extend into the opening 220 and interface with the mating side 230 of the reaction valve 202. The mating side 230 may or may not also extend into the opening 220.

The thermal body 222 is configured (e.g., shaped) so that the thermal body 222 may engage a portion of the reaction valve 202 along the mating side 230 that is associated with the flow channel 225 or the sample region 234 therein. More specifically, the thermal body 222 is located with respect to the flow channel 225 so that a temperature within the flow channel 225 may be suitably controlled by the thermal element 206. For example, in FIG. 2, the flow channel 225 is aligned along the z-axis with the thermal body 222 regardless of the rotational position of the reaction valve 202.

Accordingly, the thermal body 222 may also be configured so that the reaction valve 202 is permitted to move with respect to the base substrate 210 (e.g., rotate). FIG. 2 illustrates one embodiment of the thermal body 222 in which the thermal body 222 is annular or ring-shaped. The curvature of the thermal body 222 is similar to the curvature of the flow channel 225. When the reaction valve 202 is stacked and movably engaged to the base substrate 210, the reaction valve 202 may also be engaged to the thermal element 206. In alternative embodiments, the thermal body 222 is not ring shaped or does not form a complete circle. Instead, the thermal body 222 may only include a portion of what is shown in FIG. 2, such as an arc portion. In such embodiments, the thermal element 206 is configured to control the temperature of the flow channel 225 only when the reaction valve 202 has a particular rotational position. In some embodiments, more than one thermal element may be used. Thus, a sample in a reaction valve may experience different temperatures at different positions along its path of travel, the positions corresponding to reaction steps where a particular temperature (provided by an appropriately placed thermal element) is desired.

When the fluid-selector assembly 200 is constructed, the thermal element 206 is positioned on the main surface 205 such that the base substrate 210 is inserted through the opening 220. The reaction valve 202 is mounted to the thermal element 206 and the engagement surface 212 and arranged such that the channel outlet 240 is in fluid communication with the waste port 218. The channel inlet 238 may be in fluid communication with one of the manifold ports 216.

As shown in FIG. 2, the reaction valve 202 may include an outer or circumferential edge 250. In the illustrated embodiment, the outer edge 250 includes teeth 251 that extend radially away from the axis of rotation 290. As the reaction valve 202 is mounted onto the engagement surface 212, the outer edge 250 of the cell stage 226 may engage coupling arms 242 of the manifold 204 that have respective fingers 243. More specifically, the coupling arms 242 may be deflected away from the reaction valve 202 as the reaction valve 202 is mounted onto the base substrate 210. When the fingers 243 clear the mounting side 232 of the reaction valve 202, the coupling arms 242 may snap back into position with the fingers 243 extending over the mounting side 232.

In the illustrated embodiment, the biasing member 208 may be coupled to the mounting side 232. The biasing member 208 may be located between a housing (not shown) and the reaction valve 202 and configured to maintain a mounting force toward the manifold 204 onto the reaction valve 202. The mounting force may facilitate maintaining a sealed interface between the reaction valve 202 and the base substrate 210 along the interface.

The fluid-selector assembly 200 may also include a positioning assembly 244. The positioning assembly 244 may include a valve-engaging element 246 that directly engages the reaction valve 202 for moving the reaction valve 202 and a motor 248 that controls movement of the valve-engaging element 246 and, consequently, the reaction valve 202. In the illustrated embodiment, the valve-engaging element 246 is a rotatable component and is shaped similar to a gear having complementary teeth for engaging the outer edge 250. The motor 248 is operatively coupled to the valve-engaging element 246 and configured to rotate the valve-engaging element 246 about an axis of rotation that is parallel to the axis of rotation 290. However, a variety of actuators or gears may be used that have different shapes. As such, the valve-engaging element 246 may rotate about an axis of rotation that is not parallel to the axis of rotation 290.

The motor 248 may be a direct drive motor. However, a variety of alternative mechanisms may be used, such as direct current (DC) motors, solenoid drivers, linear actuators, piezoelectric motors, and the like. As described in greater detail below, the positioning assembly 244 is configured to move (e.g., rotate) the reaction valve 202 in order to selectively connect the flow channel 225 to one or more of the ports 216. In some embodiments, a speed at which the reaction valve 202 is rotated is sufficiently slow to place no more than negligible centrifugal force on the fluids in the reaction valve 202. Furthermore, in many cases, the reaction valve 202 is only partially rotated. For example, in some embodiments, the rotational strokes may only rotate the reaction valve 202 at most 180°. In many cases, the rotational strokes are at most 60° or less or at most 30° or less. After rotating the reaction valve 202, the reaction valve 202 is momentarily stationary to permit a fluid to flow therethrough or to permit a sample to be detected.

In the illustrated embodiment, the cell stage 226 and the flowcell 228 are separate or discrete components. For instance, the cell stage 226 and the flowcell 228 may be fabricated from different materials and then coupled together through an adhesive or fastener thereby forming a unitary structure. Thus, the flow cell 228 can optionally be a consumable, replaceable or disposable component of the fluid-selector assembly 200. Similarly, flowcells exemplified for use in other apparatus herein can be a consumable, replaceable or disposable component of a more permanent apparatus. In alternative embodiments, the cell stage 226 and the flowcell 228 are a single continuous piece. For example, the transparent material that is used to manufacture the flowcell 228 may also be used to fabricate the features of the cell stage, including the teeth 251 of the outer edge 250. Accordingly, the reaction valve 202 may be manufactured from a single component.

The cell stage 226 and the flowcell 228, as well as other flowcells and cell stages described herein, may be fabricated from a number of different materials. By way of example only, the flow cell or the cell stage may be fabricated from one or more of silica glass, phosphate glass, borosilicate glass, photosensitive glass (e.g., Foturan or Fotoform), silicon, plastic, cyclic olefin copolymers (COC) (e.g., Topas or Zeonor), doped glass (e.g. Sift doped with quantum dots, fluorescent dyes, rare earth atoms, and other atoms), methacrylate (PMMA), polycarbonate, polystyrene, polypropylene, and poly(tetrafluoroethylene) (PTFE), metal, rubber, ceramics, and the like. At least some of the above include suitable optical properties for imaging of a sample, and other may be suitable for forming a cell stage. The flowcells and cell stages may also include elastomers or other materials suitable for forming a sealable interface. Methods of fabricating flowcells that may be suitable for embodiments set forth herein are described in U.S. Patent Application Publication Nos. 2010/0111768 and 2012/0270305, each of which is hereby incorporated by reference in its entirety. International application no. PCT/US2013/30940 (filed on Mar. 13, 2013), which is incorporated herein by reference in its entirety, also describes methods of fabricating flowcells that may be suitable for embodiments described herein.

FIG. 3 is a perspective view of a cross-section of the fluid-selector assembly 200. As shown, a cross-section of the reaction valve 202 is rotated about the axis of rotation 290 about 45° with respect to the cross-section of the base substrate 210. Two of the fluid ports 216 are shown, which are referenced as 216A, 216B, and the waste port 218 is also shown. In FIG. 3, the thermal element 206 is located proximate to the sample region 234 of the flow channel 225. The thermal element 206 has a thermal-transfer surface 266 that directly engages the cell stage 226 and is generally aligned with the flow channel 225. The cell stage 226 includes a base surface 262 that is positioned adjacent to the engagement surface 212 along an interface 260. The interface 260 may also extend between the thermal-transfer surface 266 and the base surface 262. As shown, the mating side 230 of the reaction valve 202 may be shaped to surround an outer periphery of the thermal element 206 and thereby also surround the base substrate 210.

The reaction valve 202 includes valve or channel passages 252, 254. In FIG. 3, the valve passage 252 fluidly connects the flow channel 225 and the manifold port 216B, and the valve passage 254 fluidly connects the flow channel 225 and the waste port 218. More specifically, the valve passage 252 extends between a valve inlet 270 that opens to the base surface 262 and the channel inlet 238, and the valve passage 254 extends between a valve outlet 272 and the channel outlet 240. As shown, the valve passage 252 may be configured to extend the shortest possible distance between the valve inlet 270 and the channel inlet 238. In some embodiments, the valve passage 252 has a volume that is defined between the channel inlet 238 and the valve inlet 270 and is at most 10 μl or, more particularly, at most 6 μl. This volume may be referred to as the swept volume or the dead volume of the reaction valve 202 for some embodiments.

The positioning assembly 244 (FIG. 2) is configured to engage the outer edge 250 and move the reaction valve 202 in a predetermined manner. For example, the positioning assembly 244 may move (e.g., rotate) the reaction valve 202 about the axis of rotation 290 and with respect to the base substrate 210 to fluidly disconnect the valve inlet 270 from the manifold port 216B and fluidly connect the valve inlet 270 with the manifold port 216A. In a similar manner, the positioning assembly 244 may connect the valve inlet 270 with other manifold ports by changing the rotational position of the reaction valve 202. In the embodiment that is exemplified in FIG. 3, the valve outlet 272 remains aligned and fluidly connected to the waste port 218 regardless of the rotational position of the reaction valve 202. As described herein, the different rotational positions in some embodiments may be correlated to designated positions for imaging the flow channel 225. For example, in embodiments that include one objective lens, one of the areas 236 may be imaged in each rotational position. For embodiments that include multiple objective lenses, an equal number of areas 236 may be imaged.

The flow channel 225 may be located between the flowcell 228 and the cell stage 226. In the illustrated embodiment, the cell stage 226 includes a channel recess 256 and the flowcell 228 comprises an optically transparent layer that is coupled to the cell stage 226 over the channel recess 256 thereby defining the flow channel 225. As such, the flow channel 225 may be defined partially by the flowcell 228 and partially by the cell stage 226.

In other embodiments, however, the flow channel 225 or, in particular, the sample region 234 of the flow channel 225 may be defined mostly or entirely by the flowcell 228. For example, the flowcell 228 may be formed to define the flow channel within a body of the flowcell and have a pair of ports that fluidly connect to the channel inlet 238 and the channel outlet 240 when the flowcell 228 is mounted to the cell stage 226. More specifically, the flowcell 228 may include a top layer, a bottom layer, and a channel layer located between the top and bottom layers. The top and bottom layers may be continuous layers that cover the channel layer, and the channel layer may include at least one etched channel that becomes the flow channel. Flowcells that may be used with embodiments set forth herein are described in U.S. Patent Application Publication Nos. 2010/0111768 and 2012/0270305, each of which is hereby incorporated by reference in its entirety.

In alternative embodiments, the flow channel may be located a depth within the cell stage 226 and/or may be proximate to and extend along the mating side 230. Such embodiments may be suitable for applications that do not utilize an objective lens. For example, the alternative flow channel may extend along the interface 260 between the cell stage 226 and the base substrate 210. A solid-state device configured for imaging may be positioned along the interface 260. For example, the solid-state imaging device may be separate from the reaction valve 202 and the manifold 204 or may be part of one of the reaction valve 202 or the manifold 204. Fluids from the different ports of the manifold 204 may be selected in a similar manner as described herein, but the fluid may flow proximate to the interface 260 instead of the mounting side 232. Such embodiments may also be suitable for applications that do not detect optical signals or for applications that are dedicated primarily to generating a sample without monitoring the generations.

FIG. 4 is a perspective view of a system 274 that includes the fluid-selector assembly 200 and an imaging assembly 276. In order to illustrate the spatial relationship between the reaction valve 202 and the imaging assembly 276 of the system 274 in FIG. 4, the positioning assembly 244 (FIG. 2), the coupling arms 242 (FIG. 2), and the biasing member 208 (FIG. 2) are not shown. Like the system 100, the system 274 may be configured to performing biological or chemical analysis of a substance. As shown, the imaging assembly 276 includes an objective lens 278 that is positioned proximate to the flow channel 225 for imaging at least a portion of the sample region 234. For example, the objective lens 278 may have a distal surface 279 that faces the reaction valve 202. A working distance between the distal surface 279 and a surface that defines the flow channel 225 may be less than 5000 microns. The objective lens 278 may be part of an optical train (not shown) that directs optical signals from the flow channel 225 toward a detector (not shown). The objective lens 278 may have a high NA (e.g., at least about 0.6 or others described above), the imaging assembly 276 may be configured to have a resolution as described herein. In some cases, at least a portion of this optical train may also direct excitation radiation onto the sample in the reaction valve 202.

The reaction valve 202 is configured to be rotated by the positioning assembly 244 about the axis of rotation 290 to designated rotational positions. Each of the designated rotational positions may be associated with one or more operations for generating and/or analyzing the sample. For example, the reaction valve 202 may be moved to rotational positions to at least one of (a) fluidly couple an inlet of the cell stage 226 to a port of the manifold 204 or (b) position the flow channel 225 relative to the objective lens 278. Rotational positions may also be associated with other operations. For example, in other embodiments, the rotational position may locate the sample region proximate to a thermal body, such as the thermal element 206, for controlling a temperature of the flow channel 225 to facilitate one or more reactions.

In some cases, each of (a) and (b) may be accomplished with a single motive stroke. As one example, the reaction valve 202 may be in fluid communication with a first manifold port when at a first rotational position. A single motive stroke may rotate (e.g., about 30° clockwise) the reaction valve 202 from the first rotational position to the second rotational position at which time the reaction valve 202 is both fluidly coupled to a second manifold port and also positioned for imaging by the imaging assembly 276. In other words, under some circumstances, it may not be necessary to rotate the reaction valve 202 for imaging after providing a new fluid to the flow channel 225. Instead, the new fluid may be provided to the flow channel 225 and, subsequently, the flow channel 225 may then be imaged without moving the reaction valve 202 to a new rotational position. In some embodiments, the flow channel 225 may be imaged before providing the new fluid and then imaged again after providing the new fluid.

However, it is not necessary to perform more than one action or operation at a single rotational position. For example, the reaction valve 202 may be rotated multiple times in order to image different portions of the flow channel 225 without introducing a new fluid after each image. Likewise, the reaction valve 202 may be rotated multiple times to couple the flow channel 225 to a predetermined order of fluid lines without any images being captured between subsequent rotations.

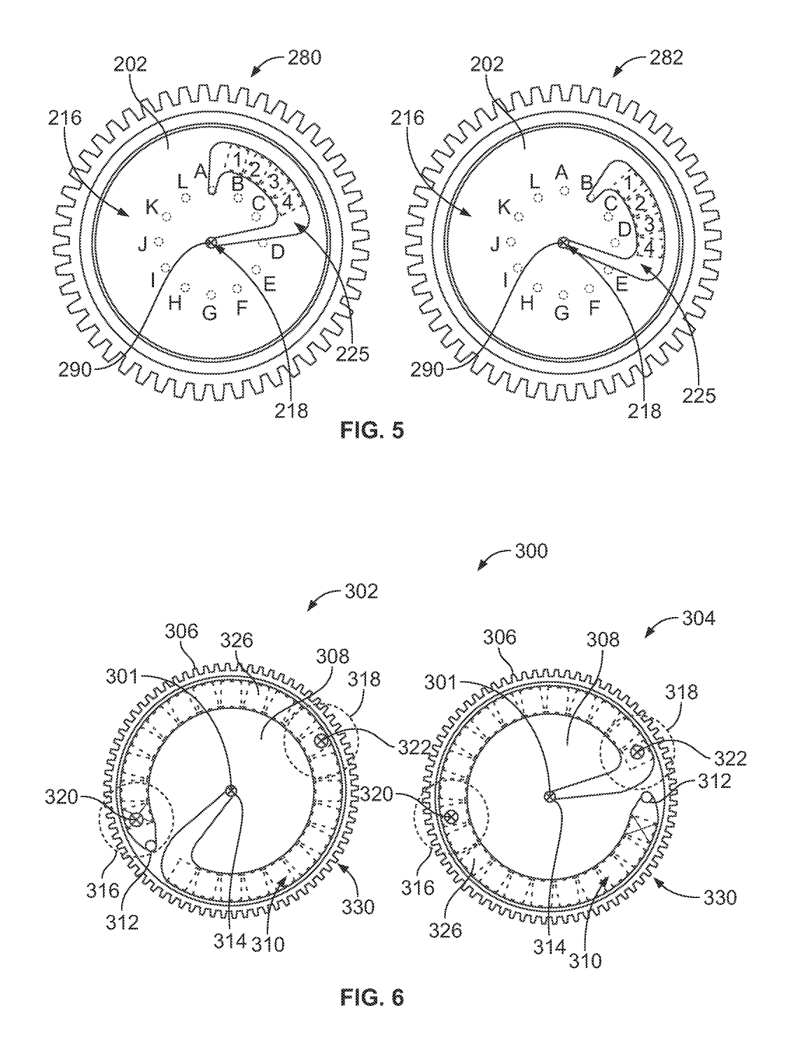

FIG. 5 is a plan view of a reaction valve 202 at first and second rotational positions 280, 282 and illustrates the different manifold ports 216 and the waste port 218 in phantom. The manifold ports 216 and the waste port 218 are located under the reaction valve 202 in FIG. 5. The manifold ports 216 have been labeled as ports A-L. As shown, the second rotational position 282 of the reaction valve 202 is about 30° clockwise with respect to the first rotational position 280.

The ports A-L may be in fluid communication with reservoirs (not shown) that contain fluids having one or more components for facilitating the designated reactions (e.g., reagents, buffer solution, analytes, etc.). In some embodiments, a one-to-one relationship exists between at least some of the ports A-L and the reservoirs. By way of example only, each of the ports A-D may be in fluid communication with only a single reservoir and each of these reservoirs may be in fluid communication with only a single port. However, in some embodiments, a single reservoir may provide fluid to multiple ports. For instance, the ports A and G may be in fluid communication with a single reservoir and, thus, may receive the same fluid. In such embodiments, a valve (not shown) may be configured to redirect the fluid to one of the ports A or G or to both of the ports A and G during operation of the system 274 (FIG. 4). In other embodiments, a single port may be in fluid communication with multiple reservoirs. Again, a valve (not shown) may be coupled to multiple fluid lines that are each in fluid communication with a different reservoir. During operation, the valve may be open for a first fluid line and closed for other fluid lines for a first period of time and then close the first fluid line and open another for a second period of time. Such a valve may also be configured to allow a plurality of fluids from a plurality of reservoirs to flow through the same port simultaneously.

As shown, the ports A-L are evenly distributed about the waste port 218 and the axis of rotation 290. For example, the ports A-L may be distributed evenly along a circle (not shown) that has the waste port 218 as the center of the circle. As such, each of the ports A-L may be located a common radial distance away from the waste port 218, but at a different circumferential location (e.g., different angle or degree of the circle). In the embodiment exemplified by FIG. 5, each of the ports A-L may be about 30° away from adjacent ports on either side.

However, the ports A-L are not required to be evenly distributed about the waste port 218. For instance, it may be useful to concentrate the ports A-L in certain regions for applications that image the flow channel 225. In particular, if the objective lens or the solid-state imaging device has a fixed position, positioning the ports A-L closer to each other may allow the flow channel 225 to be imaged more continuously or with short incremental changes while still being in fluid communication with a fluid line.

The ports A-L are also not required to be a common radial distance away from the waste port. In such embodiments, the reaction valve 202 may be both rotatable and linearly slidable. For instance, the valve inlet of the flow channel 225 may be rotated to one of the manifold ports A-L and then moved linearly to another of the manifold ports A-L. As another example in which the ports A-L are not required to be a common radial distance away from the waste port, the flow channel 225 may be in fluid communication with first and second valve inlets. More specifically, at one rotational position, the first valve inlet may be in fluid communication with the port A while the second valve inlet is not in fluid communication with any port. At another rotational position, the second valve inlet may be in fluid communication with the port B while the first valve inlet is not in fluid communication with any port.

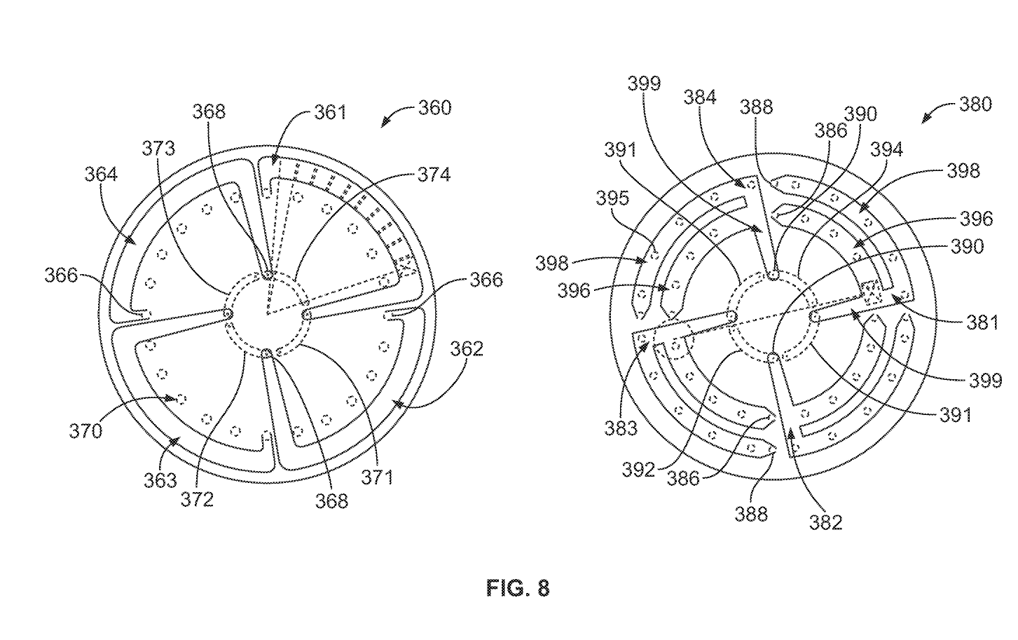

In the illustrated embodiment, the valve inlet 270 (FIG. 3) and the ports A-L have similar or identical dimensions. In an alternative embodiment, one or more of the ports A-L may be dimensioned as curved open-sided channels. For example, the port B shown in FIG. 5 may extend to and join port C. In this case, a single fluid line would provide fluid for the curved port. In such embodiments, the device inlet 270 may remain fluidly coupled to the arc-port so that fluid may continuously be provided as the reaction valve 202 rotates through a portion of its arc. Such embodiments may also allow scanning detection of the flow channel while the flow channel remains in fluid communication with the same port. However, in other embodiments, it is not necessary to flow fluid through the flow channel during imaging. In other embodiments, the waste port 218 may be an open-sided channel. Such embodiments are described with respect to the reaction valves shown in FIG. 8.

Accordingly, the valve inlet 270 (FIG. 3) is capable of fluidly connecting to any one of the ports A-L by selectively rotating the reaction valve 202 about the axis of rotation 290. As described above, the waste port 218 may remain fluidly connected to the valve outlet 272 (FIG. 3) regardless of the rotational position of the reaction valve 202.

In particular embodiments, the physical locations of the objective lens (or other appropriate component of an imaging assembly) and valve inlets are fixed relative to each other in the plane of flow cell movement and the physical locations of the ports designated areas are fixed relative to each other on the flow cell. As a result movement of the flow cell with respect to the imaging assembly and valve inlets defines the temporal order for detection of each designated area as well as the order of fluid delivery to each port on the flow cell. As such, these components can be physically located in a way that achieves a desired order for reaction and imaging steps. Furthermore, the flow cell can be moved (e.g. via rotation for a circular flow cell) at a rate that achieves a desired timeframe for each of the steps. Thus, the apparatus of the invention provides a useful device for effectively hardwiring a particular synthetic or analytical protocol into an apparatus. An advantage of particular embodiments is that a fairly complex protocol can be performed using minimal moving parts (e.g. when the only moving part is a rotating flowcell and perhaps a z-stage). However, if desired for other embodiments, relative movement can occur for several of the above components in the plane of flow cell movement in order to achieve a desired result. Whether used in a system having more or fewer moving parts, a circular flow cell is particularly useful for a repeated (i.e. cyclic) reaction since each cycle can be repeated with one lap of the flow cell and merely repeating the rotation n times can be used to carry out n cycles. As set forth in the following example, each cycle can include multiple fluidic steps and/or multiple detection steps. Table 1 below illustrates one protocol that embodiments described herein may facilitate implementing. As shown, the protocol includes a number of stages. Each stage may have one or more operations that occur during the stage. For example, Stage 1 of Table 1 is a fluidic stage in which a fluid is delivered to the flow channel 225. Stage 3 is an imaging stage in which the flow channel 225 is imaged by the imaging assembly 276 (FIG. 4). However, a stage may have more than one operation that occurs during the stage.

In particular embodiments, the imaging assembly 276 may be configured to obtain a first image of the flow channel 225 (e.g. an image of a first portion of the flow channel) when the valve inlet 270 is in fluid communication with a first manifold port and may be configured to obtain a different second image of the flow channel 225 (e.g. an image of a second portion of the flow channel) when the valve inlet 270 is fluidly disconnected from the first manifold port and, optionally, fluidly connected to a second manifold port.

Each stage may also have a designated rotational position of the reaction valve 202 or range of rotational positions for the reaction valve 202. In certain rotational positions, the flow channel 225 may be in fluid communication with one of the ports A-L. However, in other rotational positions, the flow channel 225 is not in fluid communication with any port. Such rotational positions may be used for various purposes. For example, in some cases a designated portion of the flow channel may only be imaged when the reaction valve 202 is in a rotational position in which the flow channel 225 is not fluidly connected to one of the ports. As another example, if the thermal element is localized within a predetermined region, the reaction valve 202 may be rotated to a rotational position regardless of whether the flow channel 225 is fluidly connected to a port.

A range of rotational positions may also be used during a single stage. For instance, a range of rotational positions may be used during imaging or to control the temperature within the flow channel 225 as described above. During the corresponding stage, the reaction valve 202 may be rotated continuously or incrementally to rotational positions within the range.

TABLE 1

| | | | | | |
|---|---|---|---|---|---|
| | Rotational | Port | | | |
| Type of | Position | Coupled | Type of | Temp | |
| Stage | (°) | to F.C. | Fluid | (° F.) | Time |

| | | | | |
|---|---|---|---|---|
| Stage 1 | Fluidic | 0 | A | Nucleotides |
| | | | | Solution |
| Stage 2 | Fluidic | 30 | B | Wash |
| | | | | Solution |
| Stage 3 | Image | 30-45 | — | — |
| | Area 4 | | | |
| Stage 4 | Image | 45-60 | — | — |
| | Area 3 | | | |
| Stage 5 | Image | 60-75 | — | — |
| | Area 2 | | | |
| Stage 6 | Image | 75-90 | — | — |
| | Area 1 | | | |
| Stage 7 | Fluidic | 90 | D | Cleaving |
| | | | | Solution |
| Stage 8 | Fluidic | 120 | E | Wash |
| | | | | Solution |

Repeat Stages 1-8

In each of the stages 3-6, a designated area of the flow channel 225 is imaged. The areas in FIG. 5 are referenced as areas 1-4. As shown, the designated areas 1-4 may be substantially rectangular and, as such, the areas 1-4 may be referred to as tiles. However, it is understood that the areas 1-4 may have various dimensions or geometries. In some embodiments, the imaging assembly 276 detects optical signals that are emitted from fluorescent labels in the flow channel 225 for one of the areas. As such, the imaging assembly 276 may include one or more radiation sources (e.g., lasers, LEDs, lamps, and the like) that direct an excitation radiation into the flow channel 225 to excite the fluorescent labels. In some cases, a single excitation wavelength (e.g., 532 nm) may excite multiple labels. After imaging one of the areas 1-4, the reaction valve 202 may be rotated so that a new area may be imaged.

Accordingly, the imaging assembly 276 may be capable of imaging the flow channel 225 without moving the objective lens 278 (FIG. 4) in a lateral direction along an imaging plane. More specifically, the objective lens 278 may have an optical axis that intersects the flow channel 225. The objective lens 278 may be used to image the flow channel without the objective lens 278 being moved along the xy-plane, which is orthogonal to the optical axis, between subsequent images. In order to image different portions of the flow channel 225, the positioning assembly 244 (FIG. 2) may rotate the reaction valve 202 and, thus, the flow channel 225, an arcuate distance. The positioning assembly 244 may rotate the reaction valve 202 after each of the images is obtained. Alternatively, the positioning assembly 244 may rotate the reaction valve 202 as the image(s) are being obtained. In such embodiments, the positioning and imaging assemblies 244, 276 may coordinate operations to continuously scan the flow channel 225.

FIG. 6 is a plan view of a reaction valve 300 formed in accordance with one embodiment at two different rotational positions 302, 304. The reaction valve 300 may include similar elements and features as the reaction valve 202. For example, the reaction valve 300 includes a cell stage 306 having a flowcell 308 coupled thereto. The reaction valve 300 also includes a flow channel 310 that fluidly couples a channel inlet 312 and a channel outlet 314. The channel inlet and outlet 312, 314 may be in fluid communication with a valve inlet and outlet (not shown), respectively, through corresponding valve passages (not shown). The flow channel 310 extends circumferentially around the reaction valve 300 proximate to an outer edge 330 of the cell stage 306. The flow channel 310 may encircle an axis of rotation 301 of the reaction valve 300. In FIG. 6, the reaction valve 300 in the second rotational position 304 has been rotated 160° counter-clockwise with respect to the first rotational position 302.

As indicated by phantom circles, first and second objective lenses 316, 318 of an imaging assembly (not shown) may be positioned proximate to the flow channel 310. In particular embodiments, the objective lenses 316, 318 are held in fixed positions while the reaction valve 300 is rotated along an imaging plane with respect to the objective lenses 316, 318. The imaging plane may extend orthogonal to optical axes 320, 322 of the objective lenses 316, 318, respectively.

Accordingly, embodiments set forth herein may include multiple imaging devices that are configured to image the flow channel at separate times or concurrently (e.g., at least partially overlapping time periods). FIG. 6 shows two objective lenses 316, 318, but more than two may be used in other embodiments (e.g., three, four, and more). Moreover, other embodiments may utilize solid-state imaging devices (e.g., CCD or CMOS) that are positioned immediately adjacent to the flowcell 308 instead of objective lenses. Embodiments set forth herein may be used with systems similar to those described in U.S. patent application Ser. No. 13/766,413, filed on Feb. 13, 2013, which is incorporated herein by reference in its entirety.

As shown in FIG. 6, the flow channel 310 is apportioned into several areas 326 for imaging. Each area 326 is substantially rectangular in the illustrated embodiment, but may have other geometries in other embodiments. At the first rotational position 302, the imaging assembly that includes the objective lenses 316, 318 may image a different area 326 with each of the objective lenses 316, 318. After or during the imaging for one area 326, a positioning assembly (not shown) may engage the reaction valve 300 and rotate the reaction valve 300 about the axis of rotation 301.

Similar to the embodiments described above, the channel inlet 312 may fluidly couple to one or more ports (not shown) of a manifold as the reaction valve 300 is rotated from the first rotational position 302 to the second rotational position 304 in order to receive a fluid through the corresponding port. In other embodiments, the reaction valve 300 is rotated only for positioning the flow channel 310 for imaging and the flow channel 310 does not receive any fluid between the first rotational position 302 and the second rotational position 304.

The imaging assembly may use the same image-capture parameters for each of the areas 326. For example, the imaging assembly may obtain an image of four different fluorescent signals at each area 326. In such embodiments, the reaction valve 300 may be rotated only about 180° to obtain data from all of the areas 326. Alternatively, however, the image-capture parameters associated with the objective lenses 316, 318 are different. For example, the image-capture parameters associated with the objective lens 316 may be configured to detect two types of fluorescent labels while the image-capture parameters associated with the objective lens 318 may be configured to detect two other types of fluorescent labels. In such embodiments, the reaction valve 300 may be rotated about 360° to obtain data from all of the areas 326.

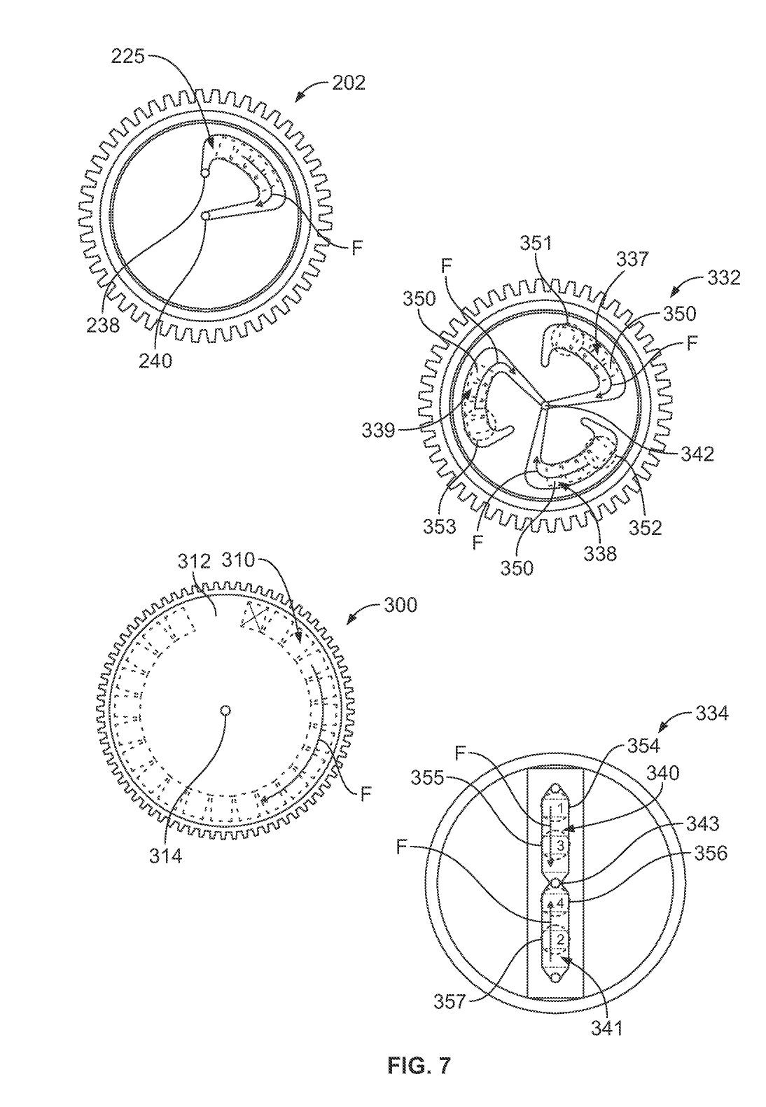

FIG. 7 includes plan views of the reaction valve 202, the reaction valve 300, and reaction valves 332, 334. Embodiments set forth herein may include or use reaction valves having a variety of configurations. For example, the reaction valves 202, 300, 332, and 334 may have different flow channel geometries and flow paths. The flow channels 225, 310, and 337-339 of the reaction valves 202, 300, and 332, respectively, are at least partially curved or arc-shaped, whereas the flow channels 340-341 of the reaction valve 334 are linear and extend radially outward (or inward) with respect to a center of the reaction valve 334. As shown in FIG. 7, each of the reaction valves 202 and 300 has a single flow channel 225, 310, respectively, in which each of the flow channels has a single channel inlet 238, 312, respectively, and a single channel outlet 240, 314, respectively.

However, the reaction valve 332 may have multiple flow channels 337-339, and the reaction valve 334 may have multiple flow channels 340-341. Each of the flow channels 337-341 has a different channel inlet, but shares a common channel outlet with another flow channel. More specifically, the flow channels 337-339 may share a common channel outlet 342, and the flow channels 340-341 may share a common channel outlet 343.

The reaction valves 332, 334 may be suitable for systems that include multiple imaging devices. For example, objective lenses 351-353 in FIG. 7 are proximate to the respective flow channels 337-339. Objective lenses 354-357 are proximate to the flow channels 340, 341. With respect to the reaction valve 332, each flow channel 337-339 has four designated areas 350 that may be scanned or imaged by a single objective lens 351-353, respectively. For example, a single designated area 350 in each of the flow channels 337-339 may be imaged and then the reaction valve 332 may be rotated for imaging another designated area 350 in each of the flow channels 337-339.

With respect to the reaction valve 334, however, each of the flow channels may be imaged by four separate objective lenses. More specifically, each of the flow channels 340, 341 is apportioned to have designated areas 1-4. When the reaction valve 334 has the rotational position as shown in FIG. 7, the flow channel 340 may have areas 1 and 3 imaged by the objective lenses 354, 355, respectively, and the flow channel 341 may have areas 4 and 2 imaged by the objective lenses 356, 357, respectively. After the first imaging operation, the reaction valve in FIG. 7 may be rotated 180°. The flow channel 341 may then have areas 1 and 3 imaged by the objective lenses 354, 355, respectively, and the flow channel 340 may have areas 4 and 2 imaged by the objective lenses 356, 357, respectively.

As shown in FIG. 7, at least some reaction valves may have flow channels with arcuate or curved channel flow paths. More specifically, a direction of fluid flow F is indicated in the flow channels 225, 310, 337-341. As shown, the reaction valves 202, 300, and 332 may have channel paths that curve about an axis of rotation. More specifically, each of the reaction valves 202, 300, and 332 may have channel paths that include substantially uniform radiuses of curvature with the axis of rotation being the center of the radius of curvature.

FIG. 8 includes plan views of reaction valves 360, 380. The reaction valve 360 includes multiple flow channels 361-364 in which each of the flow channels extends between a respective channel inlet 366 and a respective channel outlet 368. However, the flow direction may be reversed such that the channel inlet 366 becomes an outlet and the channel outlet 368 becomes an inlet. The shape of the flow channels 361-364 may be similar to the shape of the flow channel 225 (FIG. 2), although the flow channels 361-364 have a greater arcuate length than the flow channel 225. Manifold ports are shown in phantom and include fluid or component ports 370 and waste ports 371-374. Each of the waste ports 371-374 is an open-sided channel that opens to a mating side of the reaction valve 360 and has a curved or arcuate shape. In particular, the waste ports 371-374 are configured to curve along a path so that the valve outlets of the flow channels 361-364 interface with the waste ports 371-374 as the reaction valve 360 is moved (e.g., rotated). As such, each of the flow channels 361-364 remains fluidly coupled to the respective waste port 371-374 for at least a portion of the rotation of the reaction valve 360.

In the illustrated embodiment, each of the flow channels 361-364 may fluidly couple with each of the waste ports 371-374 after one complete rotation (360°) of the reaction valve 360. For example, the flow channel 361 may be in fluid communication with the waste port 371 for less than ¼(90°) of a complete rotation (e.g., about 85°), and then in fluid communication with the waste port 372 for less than ¼(90°) of a complete rotation, and then subsequently in fluid communication with the flow channels 373, 374. However, the embodiment shown in FIG. 8 of the reaction valve 360 is merely illustrative, and the number of waste ports may differ. For example, the number of waste ports may be greater than the number of flow channels. In other embodiments, the number of waste ports may be less than the number of flow channels. Furthermore, the length of the waste ports may be equal to the lengths of other waste ports as shown in FIG. 8 or the lengths of the wastes ports may be different.

With respect to the embodiments exemplified in FIG. 8, while the flow channel 361 is in fluid communication with the waste port 372, the other flow channels 362-364 may be in fluid communication with the other waste ports 373, 374, and 371, respectively. When the flow channel 361 is fluidly disconnected from the waste port 372 and fluidly connected to the next waste port 373, the other flow channels 362-364 also switch waste ports. In some embodiments, while the flow channel 361 is fluidly connected to the waste port 371, the flow channel 361 may become fluidly connected to a plurality of component ports 370. For example, the flow channel 361 may be in fluid communication with six component ports 370 while remaining in fluid communication with the waste port 371.

The reaction valve 380 also includes a plurality of flow channels 381-384 in which each of the flow channels 381-384 has a single channel outlet 390. However, each of the flow channels 381-384 also includes first and second channel inlets 386, 388. Manifold ports are shown in phantom and include two rows of fluid or component ports 395 and one row of waste ports 391-394. Each of the waste ports 391-394 is an open-sided channel that opens to a mating side of the reaction valve 380 and has a curved or arcuate shape. The flow channels 381-384 may be similarly shaped as the flow channels 361-364 of the reaction valve 360. However, the flow channels 381-384 include first and second channel branches 396, 398 that are in fluid communication with a main line 399. The reaction valve 380 and similar embodiments may provide a larger sample region.

The embodiments shown in FIG. 8 may be suitable, for example, when each of the flow channels contains a different sample for analysis. The embodiments may also be suitable for when the circumstances desire that the flow rate through the flow channels be individually controlled. For example, the flow rate through the flow channel 361 of the reaction valve 360 may be different than the flow rates in one or more of the flow channels 362-364. To this end, each of the flow channels 361-364 may be in fluid communication with a separate pumping unit or device. The flow of the fluid may be pumped through the inlets toward the outlets, or the flow of the fluid may be drawn through the outlets from the inlets.

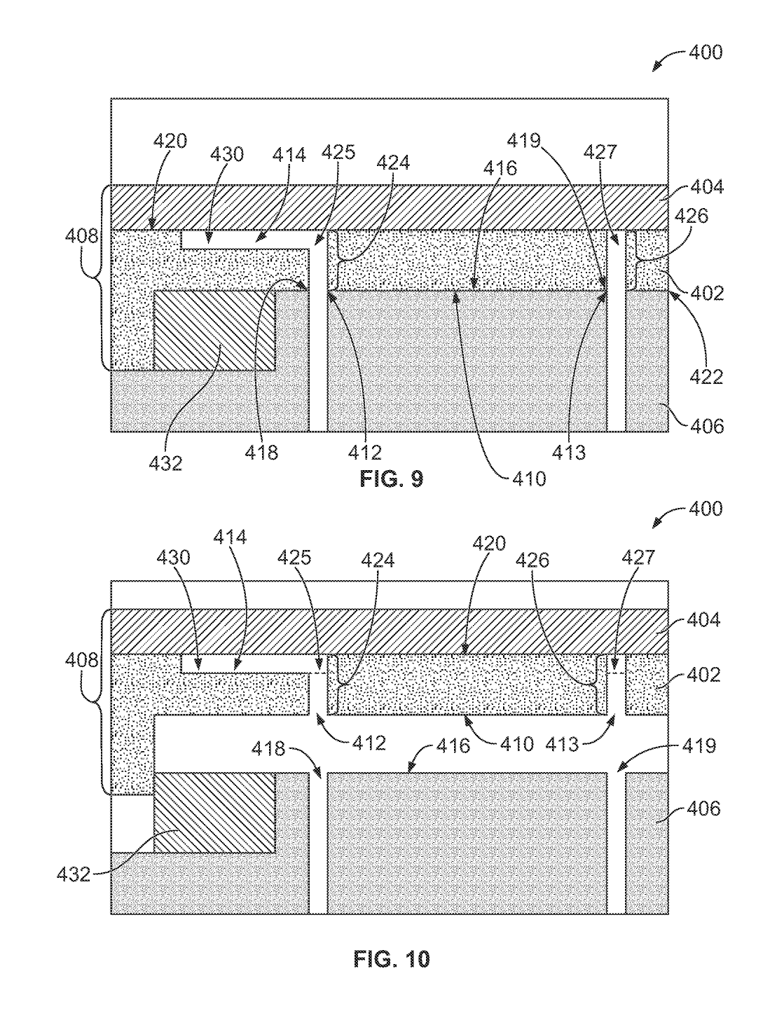

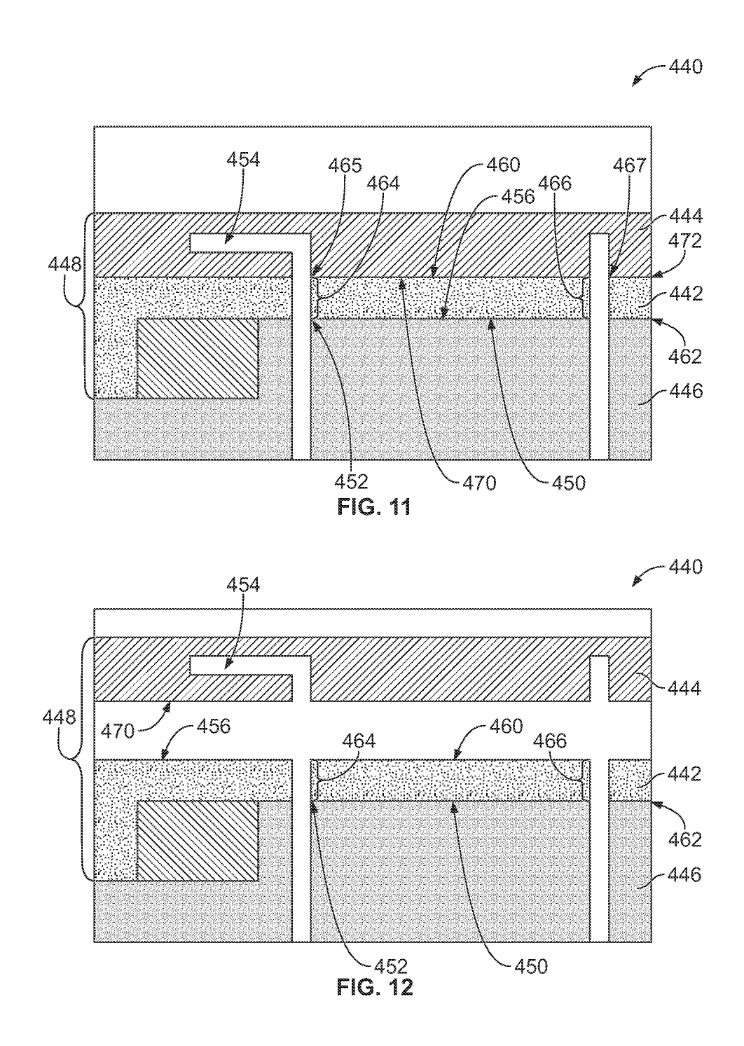

FIGS. 9-12 are side cross-sections of portions of fluid-selector assemblies. FIGS. 9-12 illustrate embodiments in greater detail and also illustrate reaction valves or flow cells being removably mounted in the fluid-selector assembly. As illustrated by FIGS. 9 and 10, the sample chamber or flow channel for at least some embodiments may be defined partially by a flowcell and partially by a cell stage. As illustrated by FIGS. 11 and 12, the sample chamber or flow channel for at least some embodiments may be defined entirely or almost entirely by a mountable flowcell. First, with respect to the fluid-selector assembly 400 shown in FIGS. 9 and 10, the fluid-selector assembly 400 includes a cell stage 402, a flowcell 404 that is coupled (e.g., mounted) to the cell stage 402, and a manifold or platform 406 that is operatively engaged to the cell stage 402.

The cell stage 402 and the flowcell 404 may be collectively referred to as a reaction valve 408. Embodiments set forth herein include reaction valves and/or flowcells that are removably coupled within the fluid-selector assembly. In such embodiments, the reaction valves and/or flowcells may be readily switched or replaced with other reaction valves/flowcells. The same manifold may be used repeatedly with a number of different reaction valves and/or flowcells. For example, in FIG. 9, the reaction valve 408 is mounted to the manifold 406. In FIG. 10, the reaction valve 408 is separated from the manifold 406, such as when the reaction valve 408 is about to be mounted onto the manifold 406 or when the reaction valve 408 is being removed from the manifold 406. As shown, the reaction valve 408 (or the cell stage 402) has a mating side 410 and a valve inlet 412 that opens to the mating side 410. The reaction valve 408 also includes a sample chamber or flow channel 414. The valve inlet 412 is in fluid communication with the sample chamber 414. The reaction valve 408 (or the cell stage 402) may also have a valve outlet 413 that opens to the mating side 410.

The manifold 406 has an engagement surface 416 and includes a manifold port 418 and a waste port 419 that open to the engagement surface 416. The engagement surface 416 and the mating side 410 of the reaction valve 408 are positioned adjacent to each other along an interface 422 (FIG. 9). Although not shown, a biasing member, such as the biasing member 208 (FIG. 2), may press the reaction valve 408 against the engagement surface 416 of the manifold 406. As described above, a positioning assembly (not shown) may be operatively coupled to at least one of the reaction valve 408 or the manifold 406 and configured to move at least one of the reaction valve 408 or the manifold 406 along the interface 422 to fluidly disconnect the valve inlet 412 from the manifold port 418 and to fluidly connect the valve inlet 412 to another manifold port (not shown). In some embodiments, the reaction valve 408 and/or the manifold 406 are rotated by the positioning assembly. In other embodiments, the reaction valve 408 and/or the manifold 406 may be moved in a linear manner by the positioning assembly.

The cell stage 402 may have a cell-receiving surface 420 and first and second passages 424, 426 that extend through the cell stage 402 and have respective ports 425, 427 located at the cell-receiving surface 420 that open to the sample chamber 414. In some embodiments, the port 425 may be referred to as a channel inlet, and the port 427 may be referred to as a channel outlet. The flowcell 404 may be coupled to the cell-receiving surface 420 in a fixed position and positioned thereon such that the ports 425, 427 are in fluid communication through the sample chamber 414.

In the illustrated embodiment, the sample chamber 414 is at least partially defined by the flowcell 404 and at least partially defined by the cell stage 402 or, more specifically, the cell-receiving surface 420. In particular, the cell-receiving surface 420 may be shaped to define a chamber recess 430. When the chamber recess 430 is covered by the flowcell 404, the chamber recess 430 may become the sample chamber 414. In some embodiments, the flowcell 404 is an optically transparent layer. The flowcell 404 may be removably mounted to the cell stage 402 or affixed to the cell stage 402. For example, an adhesive may be used to bond the flowcell 404 to portions of the cell-receiving surface 420. Also shown in FIG. 9, a thermal element 432 may be located proximate to the sample chamber 414.

Turning to FIGS. 11 and 12, the fluid-selector assembly 440 includes a cell stage 442, a flowcell 444 that is coupled to the cell stage 442, and a manifold or platform 446 that is operatively engaged to the cell stage 442. The cell stage 442 and the flowcell 444 may be collectively referred to as a reaction valve 448 when the flowcell 444 is mounted to the cell stage 442. As shown, the reaction valve 448 (or the cell stage 442) has a mating side 450 and a valve inlet 452 that opens to the mating side 450.

The cell stage 442 also has a cell-receiving surface 460 and first and second passages 464, 466 that extend through the cell stage 442 and have respective ports 465, 467 located at the cell-receiving surface 460. Also shown, the cell stage 442 may engage an engagement surface 456 of the manifold 446 along an interface 462. At least one of the cell stage 442 and the manifold 446 is movable along the interface 462.

The flowcell 444 has a coupling side 470 that engages the cell-receiving surface 460 along an interface 472. Unlike the interface 462, the flowcell 444 may be held in a fixed position with respect to the cell stage 442. However, in some embodiments, the flowcell 444 may be removably mounted to the cell stage 442 as demonstrated in FIG. 12. In such embodiments that include removable flowcells, the cell stage may be part of a system that includes manifold. As such, the cell stage and the manifold may be used repeatedly with different flowcells.

The flowcell 444 may entirely or almost entirely define a sample chamber 454 within the flowcell 444. The flowcell may be manufactured in a similar manner to the flowcells described in U.S. Patent Application Publication Nos. 2010/0111768 and 2012/0270305, each of which is hereby incorporated by reference in its entirety. For example, although not shown, the flowcell 444 may include a top layer, a bottom layer, and a channel layer that is located between the top and bottom layers. The channel layer may include at least one etched channel or slot such that, when the channel layer is sandwiched between the top and bottom layers, the etched channel becomes the sample chamber.

For embodiments in which the flowcell 444 contains the sample chamber 454, a sample may be separately prepared in the sample chamber 454 of the flowcell 444 and then the flowcell 444 may be removably mounted to the cell stage 442 for implementing the protocols described herein. Alternatively or additionally, the flowcell 444 may include reaction sites that are fabricated and patterned as described in U.S. Publication No. 2012/0316086, which is incorporated herein by reference in its entirety.

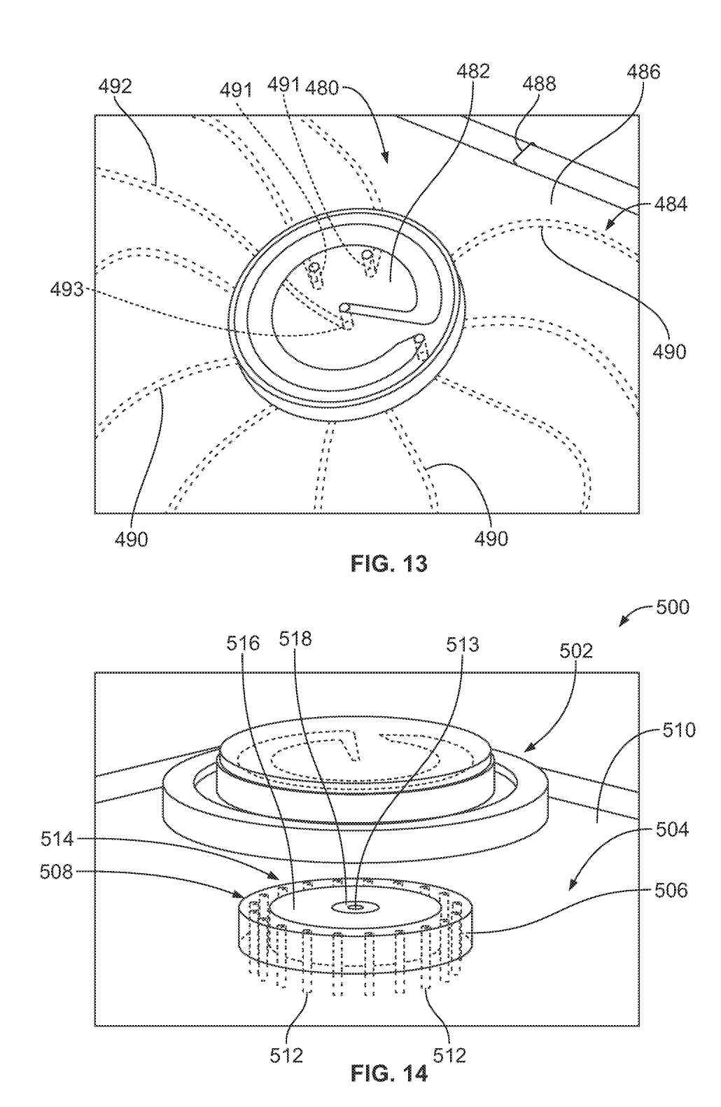

FIG. 13 is a perspective view of a portion of a fluid-selector assembly 480 that includes a reaction valve 482 that is mounted to a manifold 484. The reaction valve 482 may be similar to the reaction valve 300 (FIG. 6). The manifold 484 includes a body support 486 that is substantially planar and has a thickness 488. The manifold 484 may also include or be fluidly coupled to a plurality of incoming (or upstream) fluid lines 490 and a single outgoing (or downstream) fluid line 492. The fluid lines 490 and 492 are positioned on an opposite side of the body support 486 and are thus shown in phantom. The fluid lines 490, 492 may directly couple to manifold passages 491, 493, respectively, that extend through the manifold 484 to the reaction valve 482. The fluid lines 490, 492 may be, for examples, tubes. In other embodiments, the fluid lines 490, 492 may be formed within the body support 486. For example, such integrated fluid lines may have the length of the fluid lines 490, 492 shown in FIG. 13 but extend within the body support 486.

FIG. 14 is a perspective view of another fluid-selector assembly 500 that includes a reaction valve 502 that is configured to be coupled to a manifold 504. The manifold 504 has a base substrate 506 that includes an engagement surface 508 configured to interface with a mating side (not shown) of the reaction valve 502. The base substrate 506 may extend from a body support 510 of the manifold 504. As shown, the manifold 504 may include a plurality of manifold passages 512, 513 that extend through the body support 510 and through the base substrate 506.

The base substrate 506 may have an inner or center portion 518 and an outer (or ring) portion 520 that surrounds the inner portion 518 and defines a thermal cavity 514 therebetween. The thermal cavity 514 is sized and shaped to receive a thermal element 516. In the illustrated embodiment, the thermal cavity 514 opens to the engagement surface 508 and the thermal element 516 may be substantially flush with the engagement surface 508. The engagement surface 508 and the thermal element 516 may be substantially planar to allow the reaction valve 502 to be rotated thereon.

In FIG. 14, the thermal cavity 514 is circular (e.g., donut-shaped) and surrounds the inner portion 518 of the base substrate 506, but other geometries may be used for any of the thermal cavity 514 or the inner and outer portions 518, 520. The inner portion 518 may include the manifold passage 513. As shown, the manifold passages 512 are distributed around the thermal element 516 such that the thermal element 516 is located between the manifold passages 512 and the manifold passage 513. Each of the manifold passages 512, 513 may have a corresponding port that opens to the engagement surface 508.

In particular embodiments, the manifold passages 512 that surround the thermal element 516 are fluid passages configured to provide fluids to the reaction valve 502, and the manifold passage 513 in the inner portion 518 is a waste passage that is configured to receive an outgoing flow of fluid from the reaction valve 502. As described above, the reaction valve 502 can be selectively rotated to different rotational positions to fluidly connect the reaction valve 502 to different manifold passages 512. In the illustrated embodiment, the direction of flow is from the corresponding manifold passage 512, through the reaction valve 502, and into the manifold passage 513. In alternative embodiments, the flow may be in the opposite direction. For example, flow may be through the manifold passage 513, into the reaction valve 502, and into a selected manifold passage 512. This flow configuration may be suitable for embodiments in which a substance is generated within the reaction valve 502 and the selective rotation is used to aliquot designated volumes of the substance.

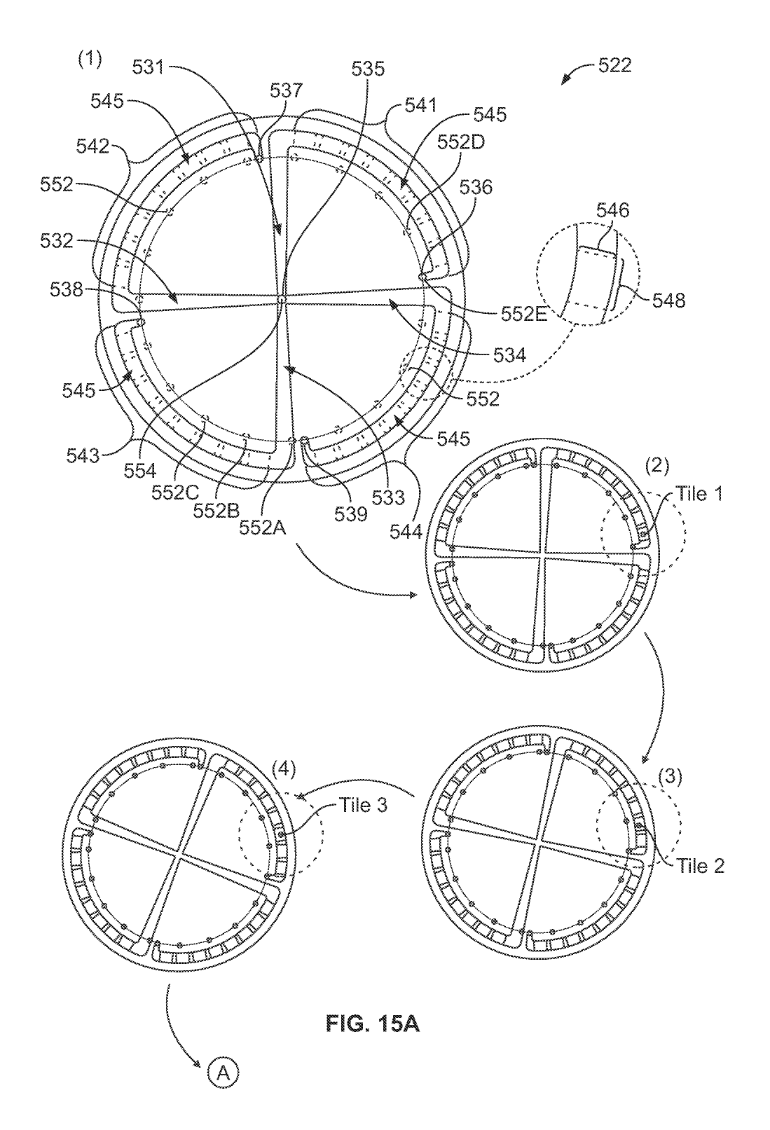

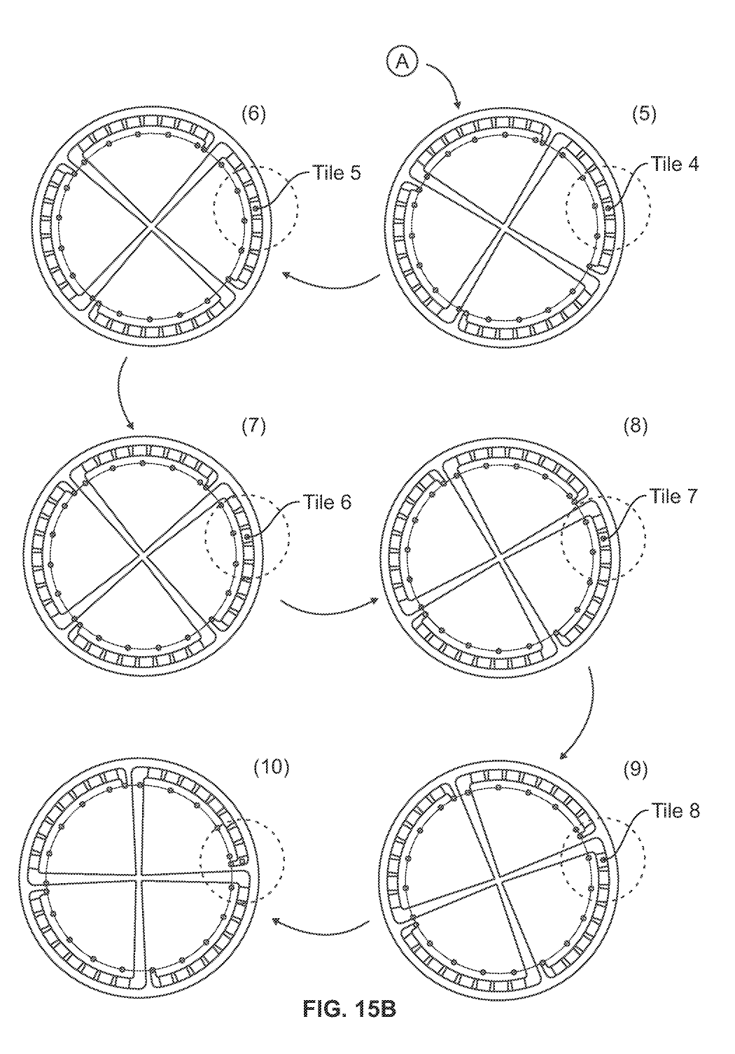

FIGS. 15A and 15B illustrate a reaction valve 522 at different rotation stages, wherein the reaction valve 522 includes four flow channels 531-534. Specifically, the reaction valve 522 is shown in the first ten rotation stages (or rotational positions) of an SBS sequencing protocol in which nucleotides are added to the DNA clusters within a sample region. The configurations shown in FIGS. 15A and 15B exemplify a physical configuration of fluidic and detection components that can carry out repeated cycles of an SBS reaction by moving only the flow cell in the x-y plane. Because the flow cell is integrated with the valve, the apparatus provides a compact design and very low dead volumes in the fluid components compared to known sequencing platforms As shown, the first ten rotation stages correlate to about ¼ of a full rotation (90°). Table 2 below provides a schedule for one complete operating cycle (e.g., one full rotation), which has thirty-six (36) rotation stages in the illustrated embodiment. Specifically, Table 2 indicates the fluid that is present in each of the flow channels 531-534 for each of the rotation stages of the reaction valve 522. The fluids marked with a (**) in Table 2 indicate that the flow channel is connected to the port that provides the marked fluid at that particular rotation stage. After the last rotation stage (i.e., rotation stage (36) in Table 2), the reaction valve 522 may return to rotation stage (1) and the schedule may be repeated. Accordingly, an embodiment may repeat the schedule shown in Table 2 numerous times (e.g., 10, 50, 100, 200, or more) during a single operating session to perform SBS sequencing in four different flow channels.

It is noted, however, that FIGS. 15A and 15B and Table 2 relate to only one particular protocol for SBS sequencing. Embodiments set forth herein may have a number of different schedules and may be used in a variety of other applications (e.g., other than SBS sequencing). Appropriate schedules and related configurations for fluidic and/or detection components can be readily determined by those skilled in the art based on the teaching and examples set forth herein.

As one example, the reaction valve 522 and the component ports can be configured for bridge amplification to form nucleic acid clusters on a surface. Useful bridge amplification methods are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420.

As another example, nucleic acid clusters in the reaction valve can be further treated to obtain a second read from the opposite end in a method known as paired end sequencing. Commercially available paired end kits and protocols are available from Illumina Inc. (San Diego, Calif.) and may be used in such embodiments. Methodology for paired end sequencing are described in PCT publication WO07010252, PCT application Serial No. PCTGB2007/003798 and US patent application publication US 2009/0088327, each of which is incorporated by reference herein. In one example, the reaction valve 522 may be rotated and the component ports 552 can be configured to accomplish the following: (a) generate clusters of nucleic acids; (b) linearize the nucleic acids; (c) hybridize a first sequencing primer and carry out repeated cycles of extension, scanning and deblocking, as set forth above; (d) “invert’ the target nucleic acids on the flow cell surface by synthesizing a complementary copy; (e) linearize the resynthesized strand; and (f) hybridize a first sequencing primer and carry out repeated cycles of extension, scanning and deblocking, as set forth above. The inversion step can be carried out be delivering reagents as set forth above for a single cycle of bridge amplification.

As shown in FIG. 15A at the rotation stage (1), each of the flow channels 531-534 are fluidly connected to a common channel outlet 535. The reaction valve 522 is configured to rotate about an axis that extends into and out of the page through the channel outlet 535. The flow channels 531-534 have respective channel inlets 536-539 and include respective sample regions 541-544, which are hereinafter referred to as sample lanes 541-544. In one or more embodiments, the sample lanes 541-544 are defined by planar surfaces (e.g., top and bottom surfaces) that extend circumferentially about the axis of rotation. For example, the top and bottom surfaces may extend continuously along the corresponding sample lane. Each of the top and bottom surfaces may be imaged or only one of the top and bottom surfaces may be imaged.

In some embodiments, a plurality of areas within a single sample lane may be sequentially imaged. The areas may be surfaces that define the sample lanes and/or spatial regions (i.e., volumes) that are defined between the surfaces. For example, each of the sample lanes 541-544 may be imaged multiple times in which each area that is imaged is located along an arcuate or circumferential path of the corresponding sample lane. For illustrative purposes, the sample lanes 541-544 include tiles 545 that are imaged according to the schedule of Table 2. Each tile represents an area (e.g., substantially two-dimensional portion of the sample lane) or region (e.g., a volume of the lane) that is imaged by the imaging assembly. One of the tiles 545 is shown in an enlarged view. The tiles 545 may be substantially rectangular having a first dimension 546 that extends substantially radially in a direction between a center of the reaction valve 522 and a perimeter of the reaction valve 522 and a second dimension 548 that extends substantially perpendicular to the first dimension 546. By way of example only, the first dimension 546 may be about 1.5 mm, and the second dimension 548 may be about 2.0 mm. As shown, each of the tiles 545 is located along an arcuate or curved path of the sample lane. Adjacent tiles may be spaced apart as shown in FIGS. 15A and 15B or, in other cases, edges of adjacent tiles may contact or overlap each other so that more area of the sample lane is imaged.

The manifold that supports the reaction valve 522 may include a plurality of component ports 552 and a waste port 554. The component ports 552 are distributed along the manifold so that the each of the channel inlets 536-539 of the respective flow channels 531-534 may, at some point, fluidly connect with each of the component ports. For example, the component ports 552 of the manifold may be located along an imaginary circle that has a center at the waste port 554. The corresponding inlet ports of the flow channels 531-534 may be located along an imaginary circle that has a center at an outlet port. Although each of the inlet ports of the flow channels 531-534 may be, at some time, fluidly connected to each of the component ports 552, the flow channels 531-534 do not necessarily receive a fluid from each of the component ports 552. For example, according to the embodiment shown in FIGS. 15A and 15B, only component ports 552A-552E provide a fluid. However, the other component ports 552 may be used during another time period, such as for sample generation. (For illustrative purposes, the component ports 552A-552E have been darkened in FIGS. 15A and 15B.

However, it is understood that the component ports 552A-552E are located under the reaction valve 522 along with other component ports 552.)

Over the course of one sequencing cycle as indicated in Table 2, each of the flow channels 531-534 receives (a) a “scan” solution, which is provided through component port 552E; (b) a “reagent” solution, which is provided through component port 552C; (c) a “cleavage” solution, which is provided through component port 552A; and (d) “wash” solutions, which are provided through component ports 552A and 552D. For example, the protocol can include a composition of fluid components and order of delivery that is used in the GA, HiSeq or MiSeq lines of DNA Sequencers from Illumina, Inc. (San Diego, Calif.). The scan solution is configured to facilitate detection of the optical signals (e.g., fluorescence) provided by the sample. For example, the scan solution may have a substantially uniform index of refraction that, in conjunction with the index of refraction of the flowcell, permits the imaging assembly (not shown) to detect the optical signals. In the context of SBS sequencing, the reagent solution includes nucleotides with fluorescent labels. The nucleotides are configured to selectively bind to sstDNA. The cleavage solution includes a deblocking reagent (e.g., enzyme) that chemically cleaves the fluorescent label and a reversible terminator from the sstDNA. The wash solution removes or dilutes components in the previous solution (e.g., the deblocking agent or the labeled nucleotides).

As shown and described in Table 2, each of the flow channels 531-534 may receive the same number of solutions and same type of solutions at different times. Nonetheless, each of the flow channels 531-534 may be subject to the same protocol. For instance, each of the flow channels 531-534 undergoes incubation periods for the same duration (albeit at different times). Each of the flow channels 531-534 may undergo a cleavage period and two wash periods for the same amount of time. Furthermore, each of the flow channels 531-534 may have an equal number of images obtained that have similar locations along the sample lanes 541-544.

TABLE 2

| Rotation | Rotational | | Fluid in | Fluid in | Fluid in | Fluid in |
|---|---|---|---|---|---|---|
| Stage | Position (°) | Tile # Imaged | FC 531 | FC 532 | FC 533 | FC 534 |
| (1) | 0.0 | N/A | Scan** | Reagent | Reagent | Scan |
| (2) | 4.5 | Tile 1 of FC 441 | Scan | Reagent | Reagent | Cleavage** |
| (3) | 14.0 | Tile 2 of FC 441 | Scan | Reagent | Reagent | Cleavage |
| (4) | 23.5 | Tile 3 of FC 441 | Scan | Reagent | Reagent | Wash** |
| (5) | 33.0 | Tile 4 of FC 441 | Scan | Reagent | Reagent | Wash |
| (6) | 42.5 | Tile 5 of FC 441 | Scan | Reagent | Reagent | Reagent** |
| (7) | 52.0 | Tile 6 of FC 441 | Scan | Reagent | Reagent | Reagent |
| (8) | 61.5 | Tile 7 of FC 441 | Scan | Reagent | Reagent | Reagent |
| (9) | 71.0 | Tile 8 of FC 441 | Scan | Wash** | Reagent | Reagent |
| (10) | 90.0 | N/A | Scan | Scan** | Reagent | Reagent |
| (11) | 94.5 | Tile 1 of FC 442 | Cleavage** | Scan | Reagent | Reagent |
| (12) | 104.0 | Tile 2 of FC 442 | Cleavage | Scan | Reagent | Reagent |
| (13) | 113.5 | Tile 3 of FC 442 | Wash** | Scan | Reagent | Reagent |
| (14) | 123.0 | Tile 4 of FC 442 | Wash | Scan | Reagent | Reagent |
| (15) | 132.5 | Tile 5 of FC 442 | Reagent** | Scan | Reagent | Reagent |
| (16) | 142.0 | Tile 6 of FC 442 | Reagent | Scan | Reagent | Reagent |
| (17) | 151.5 | Tile 7 of FC 442 | Reagent | Scan | Reagent | Reagent |
| (18) | 161.0 | Tile 8 of FC 442 | Reagent | Scan | Wash** | Reagent |
| (19) | 180.0 | N/A | Reagent | Scan | Scan** | Reagent |
| (20) | 184.5 | Tile 1 of FC 443 | Reagent | Cleavage** | Scan | Reagent |
| (21) | 194.0 | Tile 2 of FC 443 | Reagent | Cleavage | Scan | Reagent |
| (22) | 203.5 | Tile 3 of FC 443 | Reagent | Wash** | Scan | Reagent |
| (23) | 213.0 | Tile 4 of FC 443 | Reagent | Wash | Scan | Reagent |
| (24) | 222.5 | Tile 5 of FC 443 | Reagent | Reagent** | Scan | Reagent |
| (25) | 232.0 | Tile 6 of FC 443 | Reagent | Reagent | Scan | Reagent |
| (26) | 241.5 | Tile 7 of FC 443 | Reagent | Reagent | Scan | Reagent |
| (27) | 251.0 | Tile 8 of FC 443 | Reagent | Reagent | Scan | Wash** |
| (28) | 270.0 | N/A | Reagent | Reagent | Scan | Scan** |
| (29) | 274.5 | Tile 1 of FC 444 | Reagent | Reagent | Cleavage** | Scan |
| (30) | 284.0 | Tile 2 of FC 444 | Reagent | Reagent | Cleavage | Scan |
| (31) | 293.5 | Tile 3 of FC 444 | Reagent | Reagent | Wash** | Scan |
| (32) | 303.0 | Tile 4 of FC 444 | Reagent | Reagent | Wash | Scan |
| (33) | 312.5 | Tile 5 of FC 444 | Reagent | Reagent | Reagent** | Scan |
| (34) | 322.0 | Tile 6 of FC 444 | Reagent | Reagent | Reagent | Scan |
| (35) | 331.5 | Tile 7 of FC 444 | Reagent | Reagent | Reagent | Scan |
| (36) | 341.0 | Tile 8 of FC 444 | Wash** | Reagent | Reagent | Scan |

At rotation stage (1), the flow channel 531 is fluidly connected to the component port 552E, which provides the scan solution. From rotation stage (1) to rotation stage (9), the reaction valve 522 is rotated to different rotational positions to image individual tiles 545 of the flow channel 531. More specifically, the tiles 1-8 are imaged at rotation stages (2)-(9), respectively. As the sample lane 541 of the flow channel 531 is imaged during the rotation stages (2)-(9), the flow channel 534 is fluidly connected to the component ports 532A-C at rotation stages (2), (4), and (6), respectively, and each of the flow channels 532, 533 undergoes a portion of an incubation period having the reagent solution therein. At stage (10), the flow channel 532 is fluidly connected to the component port 532E and receives the scan solution. The sample lane 542 is first imaged at rotation stage (11) and is imaged at each rotation stage until rotation stage (18). As the sample lane 542 of the flow channel 532 is imaged during the rotation stages (11)-(18), the flow channel 531 is fluidly connected to the component ports 532A-C at rotation stages (11), (13), and (15), respectively, and the flow channels 533, 534 undergo a portion of the corresponding incubation periods. Table 2 provides information regarding the remainder of the operating cycle from stages (19)-(36).

As indicated in Table 2, subsequent rotation stages in which an image is obtained may differ by 9.5° (see, e.g., the rotational positions of rotation stages (2)-(9) in Table 2). The rotation of 9.5° is based in part on the viewing area of the imaging assembly (e.g., the NA of the objective lens and other factors). For each rotation stage in which an image is obtained, one or more images may be obtained. In particular embodiments, a single image of the top surface may be obtained and a single image of the bottom surface may be obtained.

To provide a specific example of a procedure that can be employed in a single operating session of the embodiment described with respect to FIGS. 15A, 15B and Table 2, each tile 545 may be imaged twice, once for the top surface and once for the bottom surface. Both images may be acquired in about one second total. As shown in Table 2, there are thirty-two images obtained in a one operating cycle for a total of about thirty-two seconds. The scan solution provided at rotation stages (1), (10), (19), and (28) may take about 0.5 seconds each for a total of about two seconds. Thus, a complete rotation of the reaction valve 522 may occur in about thirty-four seconds. Three hundred cycles may be completed in less than three hours. If each operating cycle obtains sixty-four images, each image includes a surface area of about 3.0 mm², and each 1 mm² includes about 500,000 clusters, then about 28.8 Gb may be detected in less than three hours.

During a single operating session in which three hundred operating cycles are completed, the positioning assembly, such as a motor (not shown), may rotationally actuate the reaction valve 522 more than 10,000 times or, in other words, more than 10,000 separate rotational events or strokes. The majority of these rotational strokes may be about 9.5°. However, as shown in Table 2, some of the rotational strokes may be about 4.5° or about 19.0°. The component ports 552 are positioned accordingly along the manifold.

The component ports may be located with respect to each other to allow a sufficient amount of time for the sample within the flow channel to interact with a designated fluid that has been added to the flow channel. For example, the component ports 552C and 552D are separated by about twenty-one stages. Based on the specifications of the illustrated embodiment, the locations of the component ports 552C and 552D correspond to the reagent solution incubating within the flow channel for about twenty seconds before the wash solution from the component port 552D is provided.

Accordingly, while one flow channel is being positioned for imaging, other flow channels may be exposed to conditions (e.g., fluids and/or temperatures) that are configured to achieve designated reactions, such as the extension of sstDNA in SBS sequencing. When the other flow channels have rotated around and are positioned for imaging, the sample regions in these other flow channels are prepared and the flow channels may be imaged without waiting for the designated reactions to occur. As this exemplifies, systems and apparatus of the present disclosure can provide for parallel processing of several reactions on a single reaction valve. In the case of an analytical technique, such as nucleic acid sequencing, one sample can be fluidically manipulated in a reaction valve while another sample is being detected in the same valve.

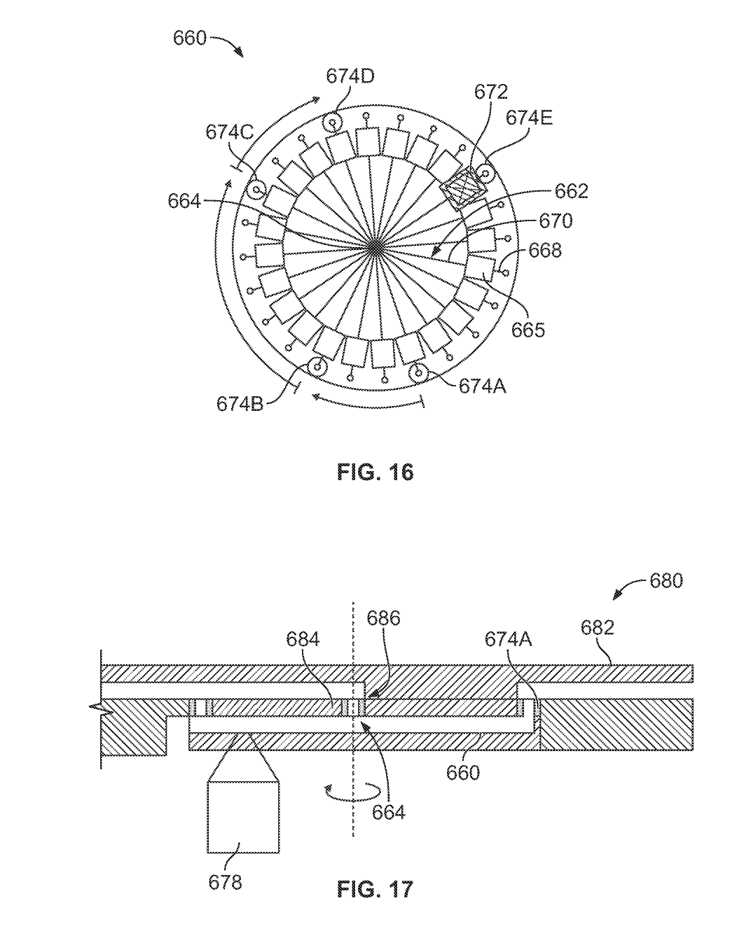

FIG. 16 is a plan view of a reaction valve 660 formed in accordance with one embodiment. The reaction valve 660 includes a plurality of flow channels 662 that extend radially from a common channel outlet 664. The flow channels 662 may include sample chambers 665 that are distributed circumferentially around the channel outlet 664 as if each of the sample chambers 665 is located along an imaginary circle that has the channel outlet 664 as its center. As shown, the flow channels 662 appear as “spokes” that extend radially between a center of the reaction valve 662 (e.g., the channel outlet 664) and a periphery of the reaction valve 622. Each of the flow channels 662 may include a flow supply line 668 and a flow drain line 670. The supply lines 668 are upstream of the sample chambers 665, and the drain lines 670 are downstream from the sample chambers 665. A direction of flow for each of the flow channels 622 is radially inward toward the channel outlet 664. Thus, the direction of flow is opposite the direction of any centrifugal forces that could be placed on fluids in the reaction valve. Typically, the speed at which the reaction valve is rotated is sufficiently slow to place no more than negligible centrifugal force on the fluids in the valve.

The sample chambers 665 are configured to be imaged by an imaging assembly 678 (FIG. 17), such as the imaging assemblies described herein. The sample chambers 665 may have different dimensions than the supply and drain lines 668, 670. For example, the sample chambers 665 may be dimensioned relative to an imaging area 672 that can be imaged by the imaging assembly. In the illustrated embodiment, the area 672 is rectangular and the sample chamber 665 is also rectangular, although with smaller dimensions. However, the sample chamber 665 may have other dimensions. For example, the dimensions may be configured to facilitate uniform fluid flow through the sample chamber 665. The reaction valve 660 is configured to be rotated by a positioning assembly (not shown) to position the sample chambers 665 within the imaging area 672.

Component ports 674A-E of a manifold 682 (FIG. 17) are also shown. The component ports 674A-E are configured to supply a reaction component to the corresponding flow channel 662 when the flow channel 662 is fluidly connected to the port. In one embodiment, the sample chambers 665 are evenly distributed about the center of the reaction valve and the imaging event for each of the sample chambers 665 requires the same amount of time (e.g., one second). In such embodiments, the reaction valve 660 may be continuously rotated in a stepwise fashion (e.g., rotate, pause for one second, rotate, pause for one second, and so on). As such, the incubation periods in which a reaction component remains in the flow channel may be based on the location of the component ports 674A-E relative to each other and the time required for image acquisition. Embodiments, such as those shown in FIG. 16, may be suitable for applications in which each sample chamber 665 receives a different sample.

FIG. 17 is a side cross-section of a system 680 formed in accordance with one embodiment that includes the reaction valve 660, the imaging assembly 678, and the manifold 682. The manifold 682 includes the component ports 674A-E, although the component port 674A is only shown in FIG. 17. In the illustrated embodiment, imaging assembly 678 is located under the reaction valve 660, which is rotatably coupled to and under the manifold 682. A gasket 684 is shown that fluidly connects a waste port 686 and the channel outlet 664. The gasket 684 may comprise an elastomeric material that seals the fluidic interconnection between the reaction valve 660 and the manifold 682.

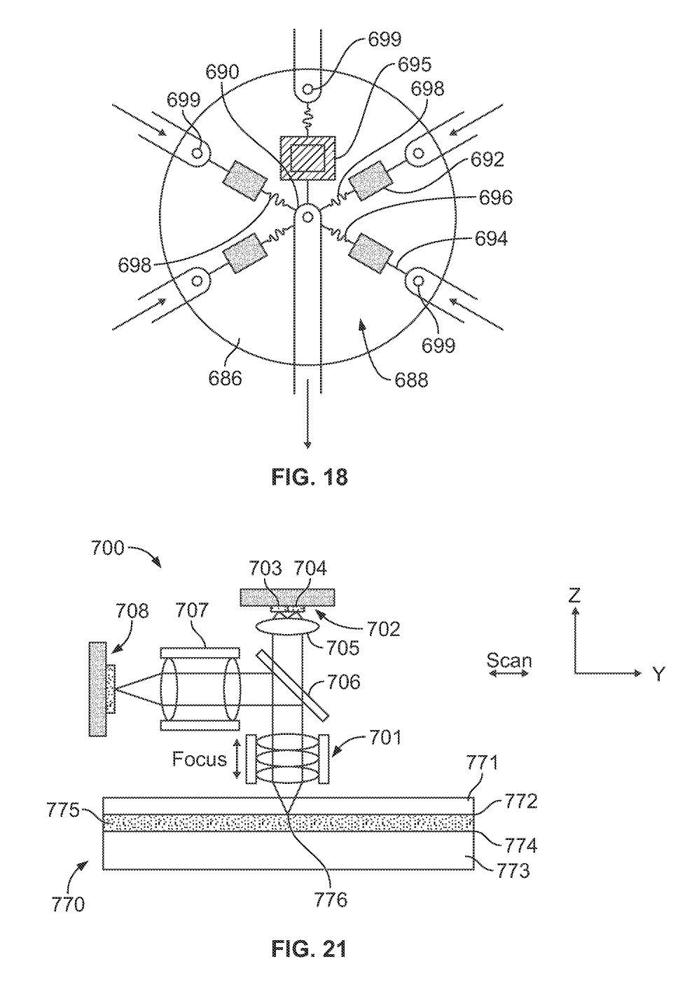

FIG. 18 is a plan view of a reaction valve 686 formed in accordance with one embodiment. The reaction valve 686 may be similar to the reaction valve 660 (FIG. 16). For example, the reaction valve 686 includes a plurality of flow channels 688 that extend radially from a common channel outlet 690. The flow channels 688 include sample chambers 692 that are distributed circumferentially around the channel outlet 690. Like the reaction valve 660, the flow channels 688 appear as “spokes” that extend radially between a center of the reaction valve 686 (e.g., the channel outlet 690) and a periphery of the reaction valve 686. Each of the flow channels 688 may include a flow supply line 694 and a flow drain line 696. The supply lines 694 are upstream the sample chambers 692, and the drain lines 696 are downstream the sample chambers 692. The sample chambers 692 are configured to be imaged by an imaging assembly (not shown), such as the imaging assemblies described herein. A rectangle shown in FIG. 18 represents an imaging area 695.

As shown, the drain lines 696 may include or be formed to provide flow restrictors 698. A flow restrictor 698 may be a portion of the drain line 696 that includes an element that obstructs fluid to impede the flow of the fluid through the corresponding sample chamber 692 or may be a portion of the drain line 696 that is dimensioned to impede the flow of the fluid through the sample chamber 692. In some embodiments, the flow restrictors 698 may be configured differently for the different flow channels 686 such that the flow rate through the flow channels 686 may be different.

A manifold (not shown) may include a plurality of component ports 699. In some embodiments, each of the component ports 699 may be pumped by a separate pumping device (not shown), such as a syringe pump or piston. Each of the pumping devices may be configured to provide, in conjunction with the corresponding flow restrictor 696, a designated flow rate through the sample chamber 692. As such, the flow rate through each flow channel 688 may be controlled and the flow rate may be different in at least some of the flow channels 688.

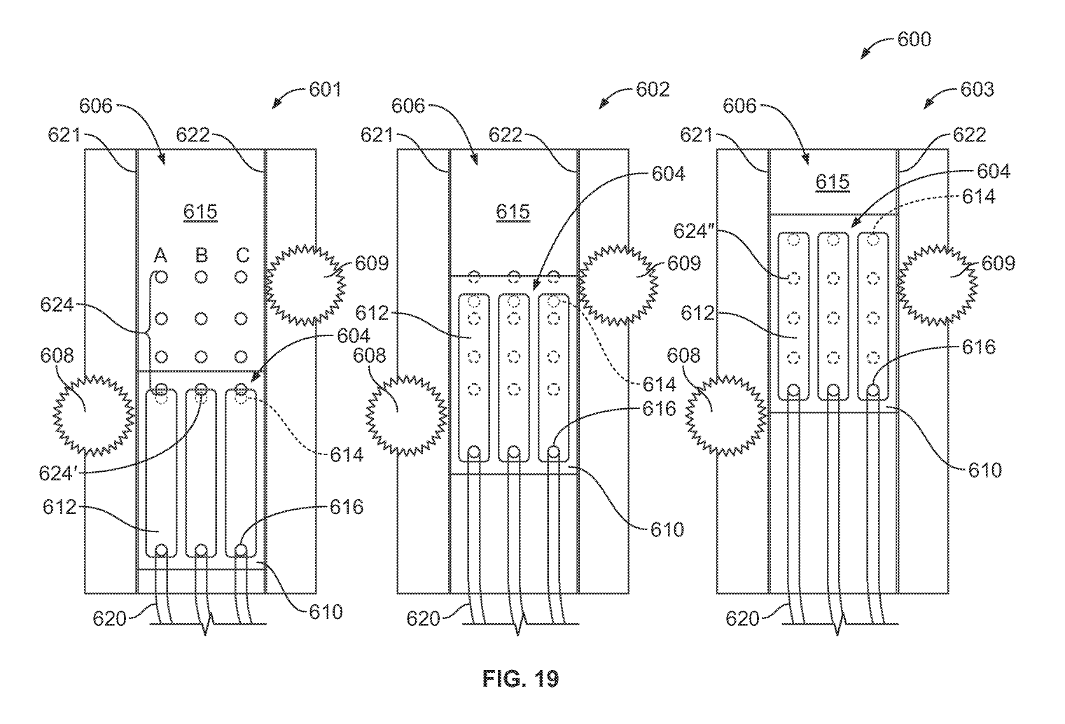

Although reaction valves are exemplified herein in the context of circular design and rotational movement, it will be understood that non-circular designs can be employed along with an appropriate direction of movement. FIGS. 19 and 20 illustrate embodiments in which at least one of the reaction valve and the manifold is moved in a linear direction. For example, FIG. 19 shows a fluid-selector assembly 600 at different translation stages 601-603. The fluid-selector assembly 600 includes a reaction valve 604, a manifold 606, and rotatable actuators 608, 609. The rotatable actuators 608, 609 are configured to rotate in opposite directions to move the reaction valve 604. The rotatable actuators 608, 609 may be engaged to motors, such as those described above. The reaction valve 604 and the manifold 606 are slidably coupled to each other. In FIG. 20, the translation stage 601 occurs before the translation stage 602, which occurs before the translation stage 603.

The reaction valve 604 includes a cell stage 610 and a plurality of flowcells 612 mounted on the cell stage 610. In the illustrated embodiment, the reaction valve 604 includes three flowcells 612 and each of the flowcells 612 extends in a linear manner and defines a flow channel between a channel inlet 614 and a channel outlet 616. In the illustrated embodiment, the channel inlets 614 of the flowcells 612 open toward an interface (not shown) between the reaction valve 604 and the manifold 606, and the channel outlets 616 open in an opposite direction away from the manifold 606. For example, the channel inlets 614 may extend through a bottom of the flowcell, and the channel outlets 616 may extend through a top of the flowcell. As shown in FIG. 19, each of the channel outlets 616 is fluidly connected to a fluid line 620.

The manifold 606 has an engagement surface 615 and may include a pair of opposing tracks or rails 621, 622 that mounted thereon. The tracks 621, 622 may define a linear path that the reaction valve 604 is configured to move along. The tracks 621, 622 engage opposite edges of the reaction valve 604. As shown with respect to the translation stage 601 in FIG. 19, the manifold 606 may include an array of manifold ports 624 that open to the engagement surface 615. The array includes three columns A-C of manifold ports 624. Each flowcell 612 is configured to align with the manifold ports 624 of a corresponding column. As such, the manifold ports 624 of each column are configured to fluidly connect with the channel inlet 614 of the corresponding flowcell 612 that is aligned with the column.

For example, the translation stage 601 shows each of the channel inlets 614 of the flowcells 612 being almost fluidly connected to a first manifold port 624′. As described herein with respect to rotatable embodiments, when the channel inlets 614 are fluidly connected to the first manifold ports 624′, then a fluid may be provided to the flow channels of the flowcells 612. At some point, the positioning assembly (not shown) may rotate the actuators 608, 609, which engage and move the reaction valve 604 in a linear direction toward the subsequent manifold ports 624. More specifically, the reaction valve 604 may slide along the interface between the reaction valve 604 and the engagement surface 615 of the manifold 606.

The positioning assembly may be configured to selectively position the reaction valve 604 relative to the manifold 606. The positioning assembly may include the actuators 608 and 609. In some embodiments, the actuators 608, 609 are configured to engage the reaction valve 604 and continue to the move the reaction valve 604 even after the other actuator is no longer engaged to the reaction valve 604. For example, the translation stage 601 may represent a starting position of the reaction valve 604. The actuator 608 at the translation stage 601 is engaged to the reaction valve 604, but the actuator 609 at the translation stage 601 is not engaged to the reaction valve 604. However, at some point after the reaction valve 604 has been moved (e.g., translated) by the actuator 608, the actuator 609 may engage and continue to move the reaction valve 604 in the linear direction.

As in other embodiments, the positioning assembly may be configured to position the reaction valve 604 so that designated areas of the flowcells 612 may be imaged. For example, at the translation stage 603, the channel inlets 614 have passed the last manifold ports 624″. However, the actuator 609 may continue to move the reaction valve 604 in the linear direction in order to position the flowcells for imaging. In some embodiments, a single objective lens (not shown) is aligned with and configured to image only one of the flowcells 612. However, alternative embodiments may include multiple objective lenses for at least one of the flowcells.

As has been exemplified for circular reaction valves, a linear reaction valve can be used for a cyclic or repeated process. Two or more steps of each cycle can be completed by movement of the linear reaction valve in a first direction. The linear reaction valve can then move in another direction (e.g. in the opposite direction) to facilitate one or more other steps of the cycle. In some embodiments, all steps of a cycle can be completed with movement of the reaction valve in one direction and a second cycle occurs as a result of a change of direction. The different steps or different cycle can occur as the linear reaction valve moves in a direction that is different from the first direction. Alternatively or additionally, the linear reaction valve can move in the different direction to a particular position and then resume movement along the first direction to repeat the two or more steps or to repeat the cycle. An advantage of a circular reaction valve is that a direction change is not necessary to repeat steps or cycles. However, it will be understood that direction changes can occur for a circular reaction valve in order to accommodate a particular schedule of fluid delivery and/or detection. The directional changes set forth above can occur whether the valve, manifold, detector or other component of the apparatus set forth herein is moved relative to other components of the apparatus.

FIG. 20 shows a fluid-selector assembly 640 at different translation stages 641-643. The fluid-selector assembly 640 includes a movable manifold 646 and a reaction valve 644 slidably mounted to an engagement surface 655 of the manifold 646. The manifold 646 is operatively engaged to a rotatable actuator 648. The actuator 648 is configured to engage and move the manifold 646 with respect to the reaction valve 644 so that channel inlets 654 of the reaction valve 644 align with a fluidly connect to ports 664 of the manifold 646. Accordingly, the reaction valve 644 may remain stationary and the manifold 646 may move with respect to it.

FIG. 21 illustrates an optical layout for an imaging assembly 700 formed in accordance with one embodiment. The imaging assembly 700 may be used with the various embodiments described herein. The imaging assembly 700 is configured to image one or more areas of a flowcell 770. The flowcell 770 has an upper layer 771 and a lower layer 773 that are separated by a fluid-filled flow channel 775. At least the upper layer 771 may be optically transparent so that the imaging assembly 700 may be focused to an area 776 on an inner surface 772 of the upper layer 771. In some embodiments, the imaging assembly 700 can be re-focused onto the inner surface 774 of the lower layer 773. One or both of the surfaces 772, 774 can include a sample region having an array of features where designated reactions occur.

The imaging assembly 700 includes an objective lens 701 that is configured to direct excitation radiation from a radiation source 702 to the flowcell 770 and to direct emission from the flowcell 770 to a detector 708. In the exemplary layout, excitation radiation from the radiation source 702 passes through a lens 705 then though a beam splitter 706 and then through the objective lens 701 on its way to the flowcell 770. In the embodiment shown, the radiation source 702 includes two light emitting diodes (LEDs) 703 and 704, which produce radiation at different wavelengths from each other. The emission radiation from the flowcell 770 is captured by the objective lens 701 and is reflected by the beam splitter 706 through conditioning optics 707 and to the detector 708 (e.g. a CMOS sensor). The beam splitter 706 functions to direct the emission radiation in a direction that is orthogonal to the path of the excitation radiation. The position of the objective lens 701 can be moved in the z dimension to alter focus of the imaging assembly 700. As described herein, the flowcell 770 may be moved transverse to the optical axis so that the imaging assembly 700 may image different areas 776 of the flowcell 770. As set forth previously herein the imaging assembly can include an optical compensator for detection of different surfaces within the flow cell (see, for example, U.S. Pat. No. 8,039,817, which is incorporated herein by reference in its entirety)

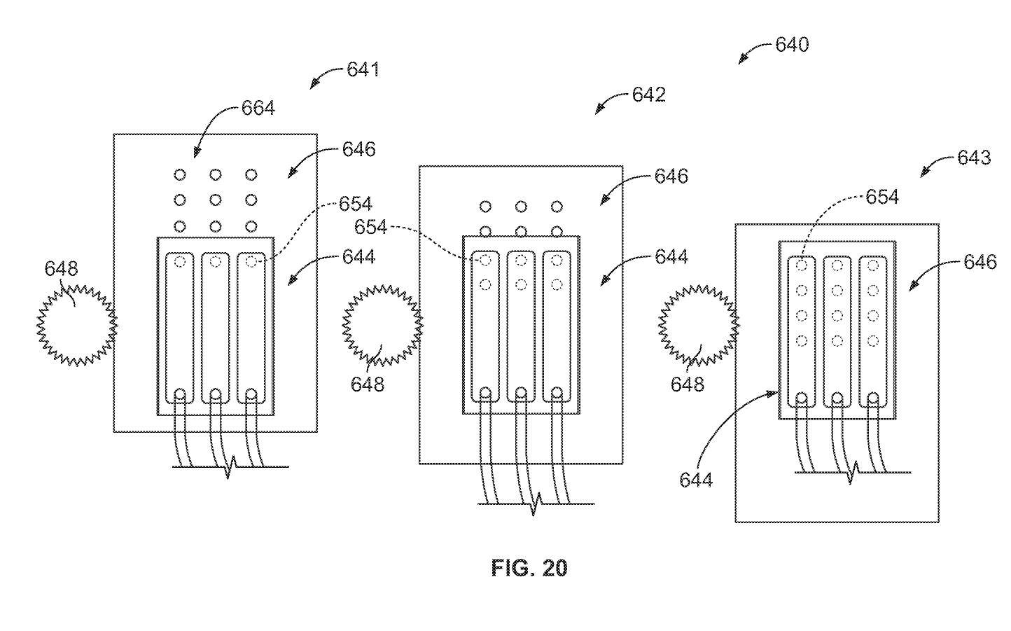

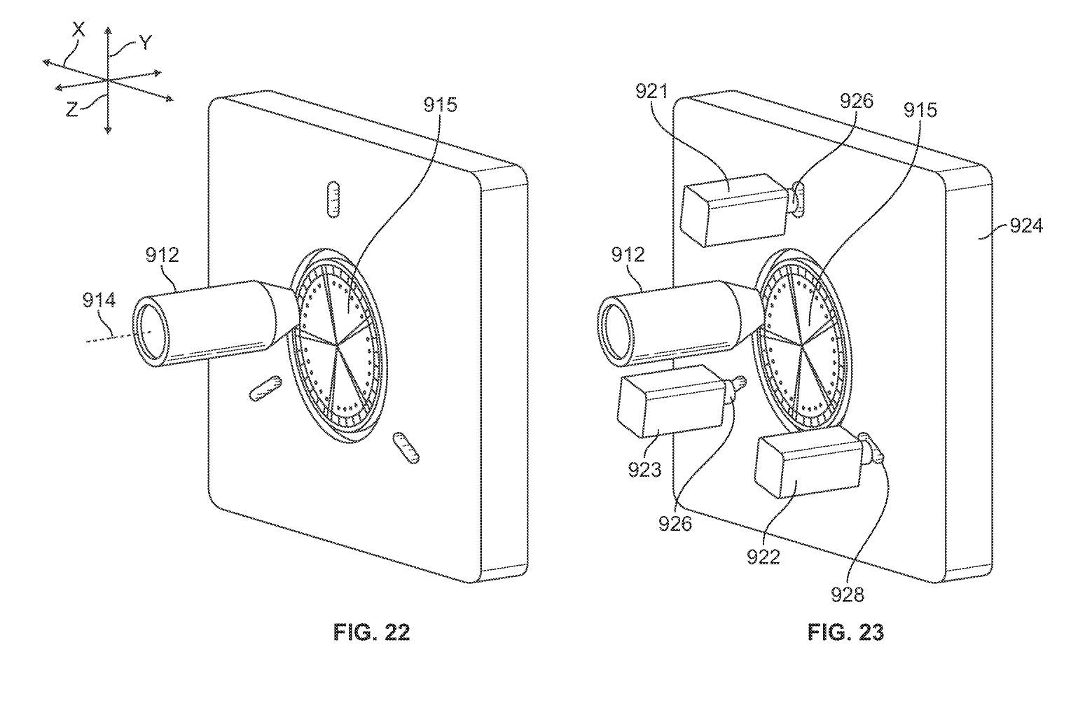

FIGS. 22-25 illustrate different alignment-control mechanisms that may be performed by the various embodiments described herein. In particular, FIGS. 22-25 demonstrate different methods of controlling the tip/tilt orientation and/or the z-position (i.e. focus position) of a reaction valve 915 having a sample region that is being imaged. When the sample region of the reaction valve 915 is imaged, the portion of the sample region that is imaged is “in focus” when the portion coincides with the imaging plane of the imaging assembly. The imaging plane is determined by the imaging assembly and, in particular, the optics in the optical train of the imaging assembly. However, due to various tolerances in the manufacturing and assembly of the reaction valve 915 and other components of the system (e.g., manifold, stage), the portion of the sample region that is imaged may not always coincide with the imaging plane of the imaging assembly.

FIGS. 22 and 23 demonstrate active mechanisms for controlling the tip/tilt alignment and/or focus of the imaging assembly. As shown in FIG. 22, an objective lens 912, which is part of the optical train, has an optical axis 914 that extends parallel to a z-axis. The imaging plane extends parallel to a plane defined by x and y axes, which are perpendicular to each other and the z-axis. The embodiment shown in FIG. 22 is configured to adjust or re-orient the imaging plane. For example, the objective lens 912 may be configured to move linearly along the z-axis (e.g., closer to and further away from the reaction valve 915), move laterally along and/or rotate about the x-axis, and move laterally along and/or rotate about the y-axis. In particular embodiments, the objective lens 912 is not moved laterally and is only movable along the z-axis or rotatable about the x or y-axes. The objective lens 915 may be moved using one or more motors, such as those described above with respect to the positioning assembly, which may be programmed to move the objective lens 912 in a designated manner to improve the alignment. The imaging assembly may also move optical components (not shown) in the optical train of the imaging assembly. For example, the imaging assembly may utilize a compensator for imaging a top surface of the flow channel. Other components of the optical train may be moved or repositioned. One or more methods or systems for moving the objective lens 915 or other optical components of the optical train or otherwise re-orienting the imaging plane may be described in U.S. Patent Appl. Publ. Nos. 2013/0023422; 2011/0220775; 2009/0272914; and 2010/0157086 and in U.S. Pat. Nos. 7,329,860 and 8,039,817, each of which is incorporated herein by reference in its entirety.

The embodiment shown in FIG. 23 utilizes stage-positioning actuators 921-923 that directly engage a stage 924 that holds the reaction valve 915. The stage 924 may be part of the manifold that directly couples to the reaction valve 915 or may hold the manifold. As shown, the actuators 921-923 include posts 926 that project along the z-axis and directly engage the stage 924. In the illustrated embodiment, the posts 926 directly engage recesses 928 that are located along the stage 924. The posts 926 are configured to move along the z-axis in a designated manner to reposition the stage 924 and thereby move the reaction valve 915. The posts 926 may also be configured to move the stage 924 along the z-axis in order to adjust the focus of the imaging assembly.

The embodiments shown in FIGS. 22 and 23 may be utilized at a predetermined frequency and/or when a predetermined event occurs. For example, the embodiments may be used each time the reaction valve 915 is loaded onto the stage 924, and/or each time a new tile is positioned for imaging, and/or each time an insufficient focus score of an image is determined by the system.

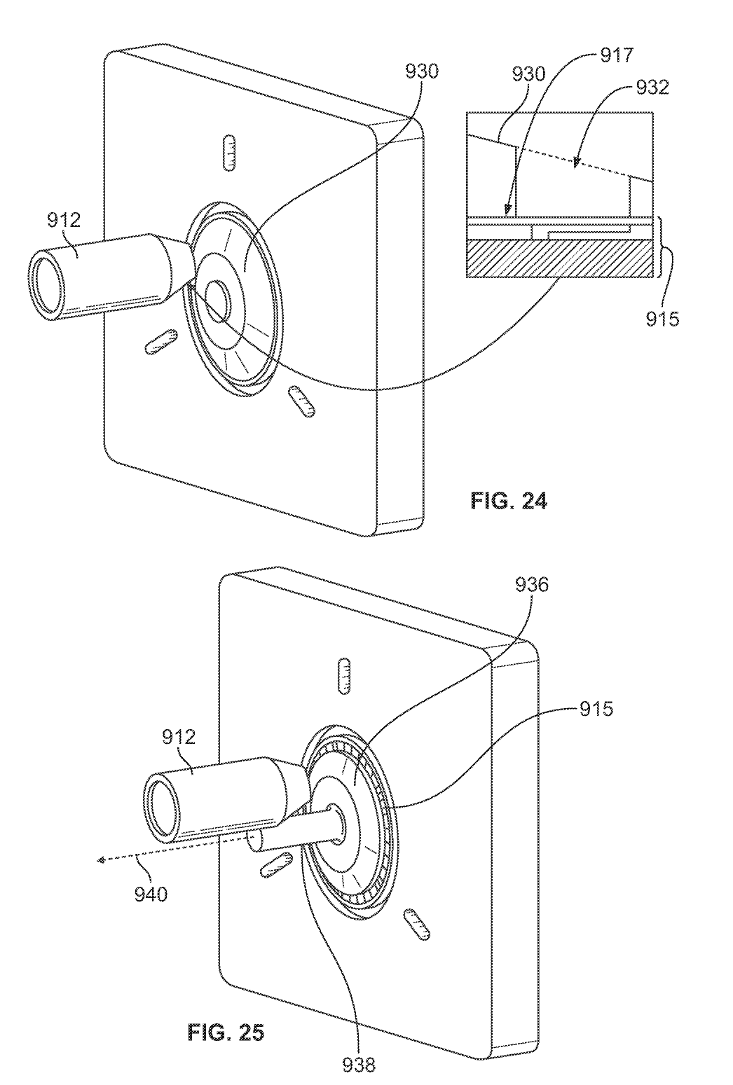

FIGS. 24 and 25 illustrate embodiments that utilize a reference element that directly engages an exterior surface of the reaction valve 915. In particular, the reference element may directly engage an exterior surface of the flowcell or the coverslip that defines the flow channel of the reaction valve 915. In some embodiments, the reference element directly engages at least three separate points of the exterior surface.

For example, with respect to FIG. 24, a reference element 930 is provided. The reference element 930 may be a cap or cover that spans across and covers at least a portion of the exterior surface of the reaction valve 915. In the embodiment of FIG. 24, the reaction valve 915 is movable with respect to the reference element 930. For example, the reference element 936 may have a fixed position (e.g., known position) with respect to the objective lens 912 and engage the reaction valve 915 along a low-friction interface 917 that permits the reaction valve 915 to be rotated. As shown in the cross-sectional view in FIG. 24, the reference element 930 may have a window 932 that permits the objective lens 912 to direct optical signals and receive optical signals therethrough. In some embodiments, the reference element 930 may also facilitate controlling a temperature of the flow channel.

In FIG. 24, the reference element 930 covers almost the entire exterior surface of the reaction valve 915. In alternative embodiments, the reference element 930 may only engage, for example, an outer rim of the reaction valve 915 or may only engage three separate points of the exterior surface.

FIG. 25 illustrates a reference element 936 that is configured to rotate with the reaction valve 915. For example, the reference element 936 may be secured to the reaction valve 915. As shown, the reference element 936 has a post or handle 938 that is configured to rotate about an axis of rotation 940 that extends parallel to the z-axis (or the optical axis of the objective lens 912). In some embodiments, the reference element 936 may be moved by the positioning assembly (e.g., a motor) and, as such, may be used as part of the positioning assembly to rotate the reaction valve 915. Like the reference element 930, the reference element 936 may have other configurations.

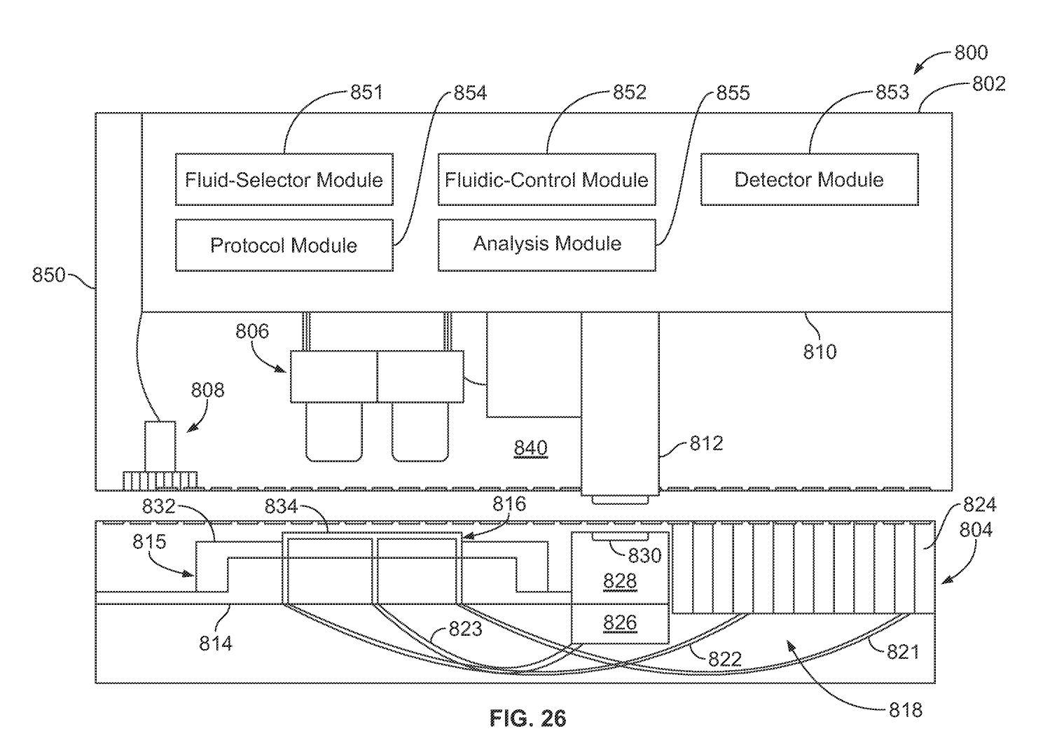

FIG. 26 is a schematic view of a system 800 configured for biological or chemical analysis formed in accordance with one embodiment. The system 800 may include one or more of the various imaging assemblies, positioning assemblies, reaction valves, and manifolds described herein. As shown, the system 800 includes a workstation 802 and a cartridge 804 that is configured to be removably loaded into the workstation 802. In the illustrated embodiment, the workstation 802 includes an imaging assembly 806, a positioning assembly 808, a computing system 810, and a pump assembly 812. The imaging assembly 806 and the positioning assembly 808 may be similar or identical to the other imaging and positioning assemblies described herein.

In the illustrated embodiment, the cartridge 804 includes a fluid-selector assembly 815 that includes a manifold 814 and a reaction valve 816 that is operatively coupled to the manifold 814. The cartridge 804 may also include a fluidic system 818 that has fluid lines 821-823, component reservoirs 824, a waste reservoir 826, and a pneumatic assembly 828 having a gas port 830 configured to engage the pump assembly 812. In some embodiments, the fluidic system 818 also includes the manifold 814 and, optionally, the reaction valve 816. Accordingly in some embodiments, the detection or imaging component of the system can be considered to be a ‘dry’ instrument as it will not have fluidic components absent the cartridge being present. The cartridge in this embodiment provides the entirety of the fluidic to the system. It will be understood that the system can however partition any number of fluidic components to one or both of the components to achieve a desired performance. Although FIG. 26 shows representative illustrations or blocks of the various components of the system 800 and general spatial relationships among the various components, it is understood that FIG. 26 is merely schematic or representative and that the system 800 may take various forms and configurations.

The reaction valve 816 includes a cell stage 832 and a flowcell 834 that is mounted to the cell stage 832. In some embodiments, the cell stage 832 is removably mounted to the manifold 814. In the same or other embodiments, the flowcell 834 may be removably mounted to the cell stage 832. The cell stage 832, the flowcell 834, and the manifold 814 may operate in a similar manner as the other cell stages, flowcells, and manifolds described herein and have similar features. For example, in the illustrated embodiment, the reaction valve 816 is rotatable about the manifold 814.

The cartridge 804 is configured to be loaded into a receiving cavity 840 of the workstation 802. For example, the cartridge 804 may be positioned under the workstation 802 as shown in FIG. 26 and then the workstation 802 may be lowered and/or the cartridge 804 may be raised so that the cartridge 804 is moved into the receiving cavity 840. The workstation 802 and/or the cartridge 804 may include one or more features or mechanisms (not shown) for guiding and aligning the cartridge with respect to the workstation 802. More specifically, when the cartridge 804 is loaded into the workstation 802, various components of the cartridge 804 and the workstation 802 may be operatively coupled to each other. More specifically: (a) the objective lenses 842, 844 may be positioned proximate to the flowcell 834 for imaging one or more sample regions of the flowcell 834; (b) the positioning assembly 808 may engage the cell stage 832 for rotating the reaction valve 816; (c) the pump assembly 812 may engage the gas port 830 for controlling/drawing fluids through the system; and, optionally, (d) a communicative connection (not shown) between the cartridge 804 and the workstation 802 may be established. With respect to the communicative connection, the cartridge 804 and the workstation 802 may communicate with each other wirelessly and/or electrical contacts (not shown) of the cartridge 804 and the workstation 802 may engage each other when the cartridge 804 is loaded.

The cartridge 804 may be disposable or at least partially disposable. For example, the cartridge 804 may include the fluidic system 818 in which the reservoirs 824 are pre-loaded with reaction components. In some embodiments, the flowcell 834 or the reaction valve 816 having the flowcell 834 may be loaded onto the manifold 814 (e.g., by a user of the system 800). The cartridge 804 may then be loaded into the workstation 802.

In some embodiments, the workstation 802 and the cartridge 804 provide the user with a “dry instrument” in which, for example, the user is not responsible for individually establishing fluidic connections or supplying the reservoirs with fluids. Instead, the system 800 may offer a more plug-and-play type capabilities in which the cartridge 804 can be loaded into the workstation 802.

The workstation 802 may be similar to a bench-top device or desktop computer. For example, at least a majority of the systems and components for conducting the designated reactions can be within a common housing 850 of the workstation 802. In other embodiments, the workstation 802 includes one or more components, assemblies, or systems that are remotely located from the workstation 802 (e.g., a remote database). The workstation 802 may include various components, assemblies, and systems (or sub-systems) that interact with each other to perform one or more predetermined methods or protocols for biological or chemical analysis.

For example, the computing system 810 may communicate with the various components, assemblies, and systems (or sub-systems) of the workstation 802. The computing system 810 may include a fluid-selector module 851, a fluidic-control module 852, a detector module 853, a protocol module 854, and an analysis module 855. Although the modules 851-855 are represented by separate blocks, it is understood that each of the modules may be hardware, software, or a combination of both and that each of the modules may be part of the same component, such as a processor. Alternatively, at least one the modules 851-855 may be part of a separate processor. Moreover, each of the modules 851-855 may be configured to communicate with each other or coordinate commands/instructions for performing a particular function.

The computing system 810 and/or the modules 851-855 may include any processor-based or microprocessor-based system, including systems using microcontrollers, reduced instruction set computers (RISC), application specific integrated circuits (ASICs), field programmable gate array (FPGAs), logic circuits, and any logic-based device that is capable of executing functions described herein. The above examples are exemplary only, and are thus not necessarily intended to limit the definition and/or meaning of the terms modules or computing system. In the exemplary embodiment, the computing system 810 and/or the modules 851-855 execute a set of instructions that are stored in one or more storage elements, memories, or modules in order to generate a sample, obtain detection data, and/or analyze the detection data.

The set of instructions may include various commands that instruct the workstation 802 to perform specific operations such as the methods and processes of the various embodiments described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

The software may be in various forms such as system software or application software. Further, the software may be in the form of a collection of separate programs, or a program module within a larger program or a portion of a program module. The software also may include modular programming in the form of object-oriented programming.

The computing system 810 is illustrated conceptually as a collection of modules, but may be implemented utilizing any combination of dedicated hardware boards, DSPs, processors, etc. Alternatively, the computing system 810 may be implemented utilizing an off-the-shelf PC with a single processor or multiple processors, with the functional operations distributed between the processors. As a further option, the modules described herein may be implemented utilizing a hybrid configuration in which certain modular functions are performed utilizing dedicated hardware, while the remaining modular functions are performed utilizing an off-the-shelf PC and the like. The modules also may be implemented as software modules within a processing unit. One or more of the computational modules can be located, for example, in a network or in a cloud computing environment.

In some embodiments, the detector module 853 may command the imaging assembly 806 to image a portion of the reaction valve 816, which may include commanding an excitation source to direct an incident light onto the portion of the reaction valve 816 to excite labels in the sample. For embodiments in which the reaction valve is moved with respect to the objective lens of the imaging assembly. The detector module 853 may obtain position data to facilitate registering the images in analysis. For example, the reaction valve, rotor, or other rotating part may include an encoder that transmits signals to the computing system 810 that are indicative of a rotational position of a component (e.g., the reaction valve or rotor). An approximate position of an image with respect to the reaction valve may be determined from the signals. In addition, one or more images obtained may include fiducial marks (or reference marks) within the image that indicate a rotational position of the reaction valve and, hence, the surface that is being imaged. The fiducial marks may be physical markings (e.g., scores, recesses, grooves, etched surfaces, embossings) that are made in the flowcell or along the surface of the cell stage. Alternatively or in addition to, the fiducial markings may include changes in color along the surface. The images may be the same sample images from which data about the sample is determined or, alternatively, a separate imaging system may be used.

In some embodiments, registration of the images is also performed by a processing unit (e.g., the detector or analysis modules). For example, in the case of SBS sequencing, each image includes numerous point sources of light from DNA clusters. Images captured from the same channel position will exhibit point sources from substantially the same locations within the image. As such, after an image is obtained, the image may be analyzed to determine or confirm the channel position of the image with respect to the reaction valve.

Although the above described methods and mechanisms of determining a position of the image are described with reference to rotating valves, the same teachings may be applied to valves that move in a linear fashion.

In alternative embodiments, in which the detection data does not include images, the detector module 853 may command detectors (not shown) to measure data of the sample at predetermined times or when predetermined conditions are satisfied.

Also shown, the fluid-selector module 851 may command the positioning assembly 808 to move one of the reaction valve 816 or the manifold 814. In the illustrated embodiment, the fluid-selector module 851 may command the positioning assembly 808 to rotate the reaction valve 816 to fluidly connect it with another manifold port and/or to position the reaction valve 816 for imaging. The fluidic-control module 852 may command the various pumps and valves of the system 800 to control a flow of fluids, such as the pump assembly 812 and/or the pneumatic assembly 828. As an example, the fluidic-control module 852 may command the pump assembly 828 to draw fluid through the waste port of the manifold 814.

The protocol module 854 may include instructions for coordinating the operations of the system 800 so that a designated protocol may be executed. The protocol module 854 may also be configured to command any thermal control elements to control a temperature of the fluid. By way of example only, protocol module 854 may be a sequencing-by-synthesis (SBS) module configured to issue various commands for performing sequencing-by-synthesis processes. In some embodiments, the protocol module 854 may also process detection data. After generating the amplicons through bridge PCR, the protocol module 854 may provide instructions to linearize or denature the amplicons to make sstDNA and to add a sequencing primer such that the sequencing primer may be hybridized to a universal sequence that flanks a region of interest. Each sequencing cycle extends the sstDNA by a single base and is accomplished by modified DNA polymerase and a mixture of four types of nucleotides delivery of which can be instructed by the protocol module 854. The different types of nucleotides have unique fluorescent labels, and each nucleotide has a reversible terminator that allows only a single-base incorporation to occur in each cycle. After a single base is added to the sstDNA, the protocol module 854 may instruct a wash step to remove nonincorporated nucleotides by flowing a wash solution through the flowcell. The protocol module 854 may further instruct an excitation source assembly and the imaging assembly to perform an image session(s) to detect the fluorescence in each of the four channels (i.e., one for each fluorescent label). After imaging, the protocol module 854 may instruct delivery of a deblocking reagent to chemically cleave the fluorescent label and the terminator from the sstDNA. The protocol module 854 may instruct a wash step to remove the deblocking reagent and products of the deblocking reaction. Another similar sequencing cycle may follow.

Exemplary protocol steps that can be coordinated by protocol module 854 include fluidic and detection steps used in reversible terminator-based SBS methods, for example, asset forth herein or described in US Patent Application Publication No. 2007/0166705 A1, US Patent Application Publication No. 2006/0188901 A1, U.S. Pat. No. 7,057,026, US Patent Application Publication No. 2006/0240439 A1, US Patent Application Publication No. 2006/0281109 A1, PCT Publication No. WO 05/065814, US Patent Application Publication No. 2005/0100900 A1, PCT Publication No. WO 06/064199 and PCT Publication No. WO 07/010251, each of which is incorporated herein by reference in its entirety. Exemplary reagents for reversible terminator-based SBS are described in U.S. Pat. No. 7,541,444; U.S. Pat. No. 7,057,026; U.S. Pat. No. 7,414,116; U.S. Pat. No. 7,427,673; U.S. Pat. No. 7,566,537; U.S. Pat. No. 7,592,435 and WO 07/135368, each of which is incorporated herein by reference in its entirety. Protocol steps and reagents used in commercial sequencing platforms such as the GA, HiSeq and MiSeq platforms from Illumina, Inc. (San Diego, Calif.) can also be used.

In some embodiments, the protocol module 854 may be configured to issue various commands for performing the steps of a pyrosequencing protocol. Exemplary steps include those set forth below and in the references cited below. Pyrosequencing detects the release of inorganic pyrophosphate (PPi) as particular nucleotides are incorporated into the nascent strand (Ronaghi, M. et al. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M. et al. (1998) “A sequencing method based on real-time pyrophosphate.” Science 281(5375), 363; U.S. Pat. No. 6,210,891; U.S. Pat. No. 6,258,568 and U.S. Pat. No. 6,274,320, the disclosures of which are incorporated herein by reference in their entireties. In pyrosequencing, released PPi can be detected by being immediately converted to adenosine triphosphate (ATP) by ATP sulfurylase, and the level of ATP generated is detected via luciferase-produced photons. In this case, the reaction valve 816 may include millions of wells where each well has a single capture bead having clonally amplified sstDNA thereon. Each well may also include other smaller beads that, for example, may carry immobilized enzymes (e.g., ATP sulfurylase and luciferase) or facilitate holding the capture bead in the well. The protocol module 854 may be configured to issue commands to run consecutive cycles of fluids that carry a single type of nucleotide (e.g., 1st cycle: A; 2nd cycle: G; 3rd cycle: C; 4th cycle: T; 5th cycle: A; 6th cycle: G; 7th cycle: C; 8th cycle: T; and on). When a nucleotide is incorporated into the DNA, pyrophosphate is released thereby instigating a chain reaction where a burst of light is generated. The burst of light may be detected by the detector assembly. Detection data may be communicated to the analysis module 855 for processing.

In some embodiments, the user may provide user inputs through the user interface to select an assay protocol to be run by the system 800. In other embodiments, the system 800 may automatically detect the type of reaction valve 800 that has been inserted into the workstation 802 and confirm with the user the assay protocol to be run. Alternatively, the system 800 may offer a limited number of assay protocols that could be run with the determined type of reaction valve 816. The user may select the desired assay protocol, and the system 800 may then perform the selected assay protocol based on preprogrammed instructions.

The analysis module 855 may be configured to analyze detection data that is obtained by the system 800. Although not shown, the workstation 802 may also include a user interface that interacts with the user. For example, the user interface may include a display to display or request information from a user and a user input device to receive user inputs. In some embodiments, the display and the user input device are the same device (e.g., touch-sensitive display).

In some embodiments, nucleic acids can be attached to a surface and amplified prior to or during sequencing. Protocol module 854 can include instructions for the fluidic steps involved in an amplification process. For example, instructions can be provided for a bridge amplification technique used to form nucleic acid clusters on a surface. Useful bridge amplification methods are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420. Another useful method for amplifying nucleic acids on a surface is rolling circle amplification (RCA), for example, as described in Lizardi et al., Nat. Genet. 19:225-232 (1998) and US 2007/0099208 A1, each of which is incorporated herein by reference. Emulsion PCR on beads can also be used, for example as described in Dressman et al., Proc. Natl. Acad. Sci. USA 100:8817-8822 (2003), WO 05/010145, or U.S. Patent Publ. Nos. 2005/0130173 or 2005/0064460, each of which is incorporated herein by reference in its entirety.

In some embodiments, the system 800 is configured to be operated with minimal user intervention. For example, the generating and analyzing operations may be conducted in an automated manner by an assay system. In some cases, a user may only load the cartridge 804 and activate the workstation 802 to perform the protocol.

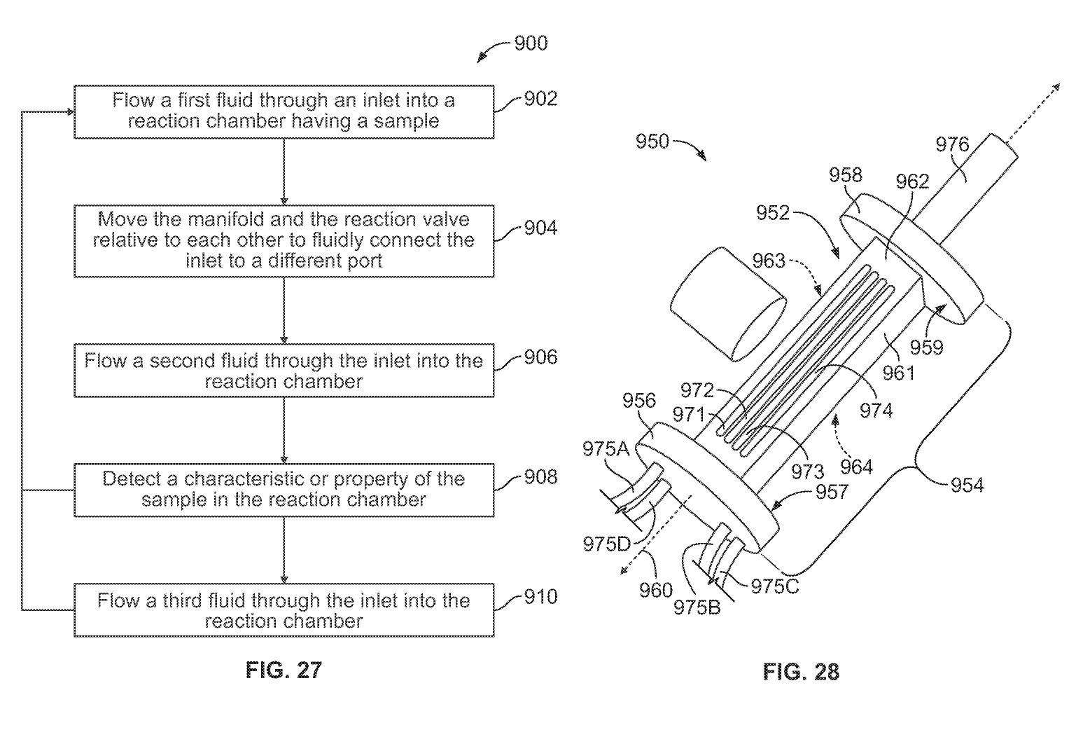

FIG. 27 is a flowchart of a method 900 of at least one of generating or analyzing a biological or chemical sample using a reaction valve that includes a sample chamber. The reaction valve may be positioned adjacent to a manifold along an interface between the reaction valve and the manifold. The reaction valve may have a valve inlet, and the manifold may have a plurality of ports. The reaction valve and the manifold may be similar or identical to the reaction valves and manifolds described herein.

The method 900 may include flowing at 902 a first fluid into the sample chamber, such as a flow channel. The first fluid may flow through a first port of the manifold before the sample chamber. The first fluid may include a reaction component. In particular embodiments, the reaction component is a labeled nucleotide having, optionally, a reversible terminator. The method 900 may also include moving at 904 the manifold and the reaction valve relative to each other along the interface to fluidly disconnect the valve inlet from the first port and to fluidly connect the valve inlet with a second port. The method may also include flowing at 906 a second fluid through the second port and into the sample chamber. The second fluid may include another reaction component or may include a wash solution. At 908, the sample chamber may be imaged. In particular embodiments, a surface that defines the sample chamber may be imaged.

The operations 902, 904, 906, and 908 may be repeated a plurality of times. For example, the operations may be completed to conduct a nucleic acid sequencing protocol. After the imaging operation 908, the method 900 may include flowing at 910 a third fluid into the sample chamber to remove a component of the sample. For example, the third fluid may include an enzyme for cleaving the reversible terminator. In some cases, the imaging operation 908 may include imaging a first area in the sample chamber, moving the manifold and the reaction valve relative to each other, and imaging a second area in the sample chamber.

FIGS. 28-32 illustrate additional embodiments of fluid-selector assemblies. In various implementations, such as those described above, the surface or region that is detected (e.g., imaged) extends along a plane that is orthogonal to the axis of rotation of the reaction valve. FIGS. 28-32 illustrate embodiments in which the surface or region that is detected extends along a plane that is parallel to an axis of rotation. For example, FIG. 28 is a perspective view of a fluid-selector assembly 950 formed in accordance with one embodiment. The fluid-selector assembly 950 includes a reaction valve 952 and a manifold 954 having first and second base substrates 956, 958. The first and second base substrates 956, 958 are spaced apart from each other with respective engagement surfaces 957, 959 that face each other. The reaction valve 952 is configured to extend between and engage the engagement surfaces 957, 959 of the base substrates 956, 958.

The reaction valve 952 is configured to rotate about an axis of rotation 960 that extends through the reaction valve 952. The reaction valve 952 may have at least one exterior side that faces radially away from the axis of rotation 960 and includes at least one sample chamber or flow channel therealong. For example, the reaction valve 952 includes four exterior sides 961-964 that each has four flow channels 971-974. As such, in the illustrated embodiment, the reaction valve 952 has a rectangular shape. However, in other embodiments, the reaction valve 952 may have any suitable number of exterior sides with any suitable number of flow channels. For example, the reaction valve 952 may have a substantially cylindrical shape such that the reaction valve 952 has only a single, continuous exterior side. In addition, a cross-section of the reaction valve 952 taken transverse to the axis of rotation 960 may have any polygonal shape. For example, the cross-section may be a triangle, quadrilateral (e.g., rectangle or square as shown in FIG. 30), pentagon, hexagon, heptagon, octagon, 9-gon, 10-gon, et seq. The exterior sides of the cross-section may be planar or have a designated curvature. In the illustrated embodiment, the exterior sides 961-964 have the same dimensions. In other embodiments, at least two exterior sides may be different.

In the illustrated embodiment, the flow channels 971-974 are linear and extend parallel to each other. More specifically, the flow channels 971-974 are elongated with the greatest dimension extending parallel to the axis of rotation 960. However, flow channels may have a variety of dimensions. For example, in other embodiments, the flow channels may have paths that are non-linear. The flow channels may have paths that are, for at least one portion thereof, skew to the axis of rotation 960. In some embodiments, the greatest dimension may be measured along a line that is skew to the axis of rotation 960.

The first and second base substrates 956, 958 may remain stationary as the reaction valve 952 is rotated. The reaction valve 952 has opposite mating sides that slidably engage the engagement surfaces 957, 959 along respective interfaces as the reaction valve 952 is rotated. Similar to the fluid-selector assemblies described above, the reaction valve 952 and the first and second base substrates 956, 958 may have respective ports. For example, ports 1001-1004 of the first base substrate 956 are shown in FIG. 30, and ports 1011-1014 of the reaction valve 952 are shown in FIG. 30. The ports 1011-1014 are rotated with the reaction valve 952 and are configured to selectively connect with the ports 1001-1004 of the base substrate 956 based on the rotational position of the reaction valve 952. In a similar manner, ports (not shown) of the reaction valve 952 may fluidly connect to ports (not shown) of the base substrate 958.

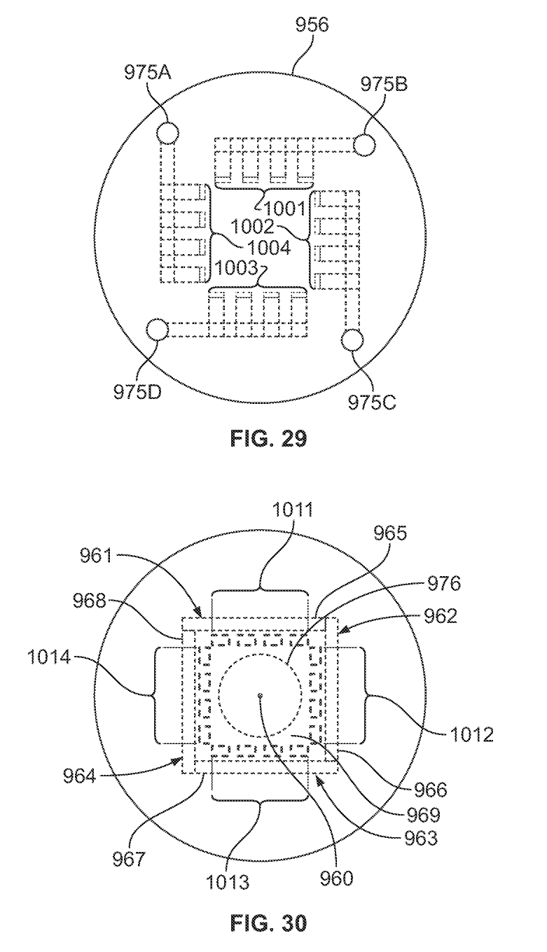

As shown in FIG. 28, fluid lines (e.g., tubes) 975A-975D may couple to the base substrate 956. A waste line 981 may couple to the base substrate 958. Each of the fluid lines 975A-975D may provide a different reaction component to the reaction valve 952. Accordingly, the base substrates 956, 958 may have passages that interconnect the fluid lines and flow channels. For example, a solution that flows through the fluid line 975A may flow into the first base substrate 956 and be divided by four passages (shown in FIG. 29) that branch out from each other and fluidly connect with one of the flow channels depending on the rotational position of the reaction valve 952. As shown in FIG. 29, the fluid line 975A fluidly connects to the ports 1004, the fluid line 975B fluidly connects to the ports 1001, the fluid line 975C fluidly connects to the ports 1002, and the fluid line 975D fluidly connects to the ports 1003.

As shown in FIG. 28, the reaction valve 952 may be operably connected to a rotor 976 that is configured to rotate about the axis of rotation 960. In the illustrated embodiment, the rotor 976 is an elongated cylinder that has the axis of rotation 960 extending through a geometric center thereof. The rotor 976 may be operably coupled to a motor (not shown) that rotates the rotor 976.

FIGS. 29 and 30 are end views of the flow-selector assembly 950. FIG. 29 shows the base substrate 956 with the ports 1001-1004 in phantom. FIG. 30 is an end view of the base substrate 956 with the reaction valve 952 in phantom. As shown in FIG. 30, the reaction valve 952 includes flowcells 965-968, which may include the exterior sides 961-964, respectively, of the reaction valve 952. Similar to other flowcells described herein, each of the flowcells 965-968 may entirely define the flow channels 971-974 or the flowcells 965-968 may be coverslips that define the flow channels 971-974 with surfaces of a cell stage 969. As shown, the rotor 976 is connected to the cell stage 969.

Returning to FIG. 28, the reaction valve 952 may be selectively rotated to (a) selectively connect the flow channels 971-974 to the fluid lines 975A-975D and (b) position the flow channels 971-974 with respect to an objective lens 982. In the illustrated embodiment, the objective lens 982 is capable of moving along a plane that the exterior side 963 extends along. After imaging the flow channels of the exterior side 963, the reaction valve 952 may be rotated to position the flow channels of another exterior side.

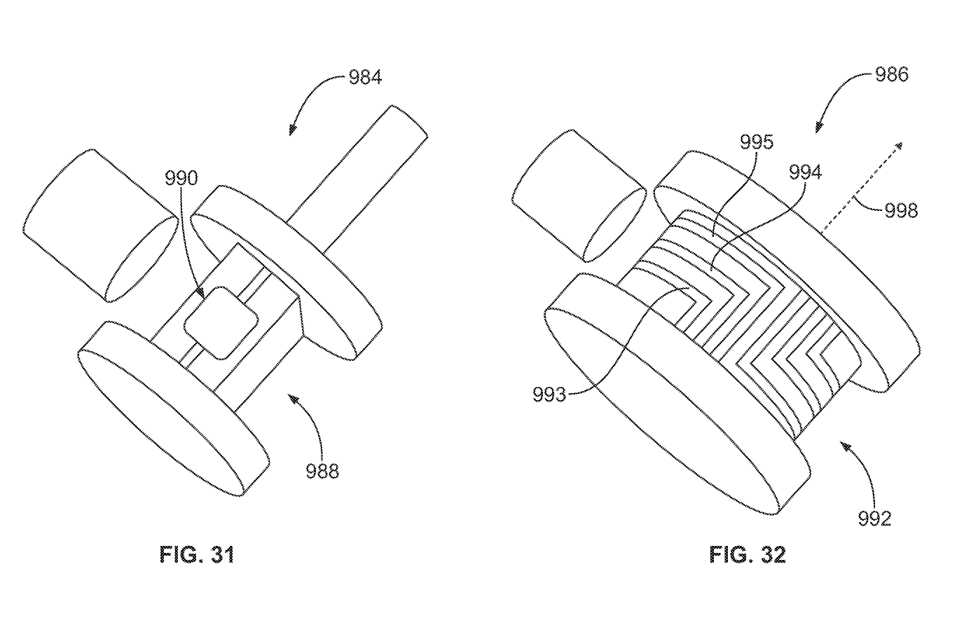

FIGS. 31 and 32 illustrated perspective views of flow-selector assemblies 984 and 986, which may be similar to the flow-selector assembly 950. As shown in FIG. 31, the flow-selector assembly 984 includes a reaction valve 988 having a flow channel 990. The flow channel 990 may be similar to the flow channels 662 described above and include a supply line, sample chamber, and drain or waste line. The sample chamber may be rectangular-shaped and may be sized according to the field of view of the objective lens.

FIG. 32 illustrates a reaction valve 992 that is substantially cylindrical or disc-shaped and has flow channels 993-995. The flow channels 993-995 may extend circumferentially around an axis of rotation 998. In some embodiments, the radius of curvature of the channel surface that is imaged by the imaging assembly may be sufficient such that the entire field of view may be in focus. In other embodiments, the imaging assembly may scan a line of the flow channel as the reaction valve 992 is rotated.

As used herein, an element or step recited in the singular and proceeded with the word “a” or “an” should be understood as not excluding plural of said elements or steps, unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means plus-function format and are not intended to be interpreted based on 35 U.S.C. §112, sixth paragraph, unless and until such claim limitations expressly use the phrase “means for” followed by a statement of function void of further structure.

1-33. (canceled)

34. A system comprising:
a reaction valve including a sample chamber;
a positioning assembly operatively coupled to the reaction valve and configured to rotate the reaction valve about an axis of rotation; and
an imaging assembly positioned proximate to the sample chamber of the reaction valve and configured to obtain at least one image of the sample chamber, the positioning assembly configured to rotate the reaction valve to move the sample chamber relative to the imaging assembly, the positioning assembly configured to at least one of (a) rotate the reaction valve after the imaging assembly has imaged the sample chamber or (b) rotate the reaction valve as the imaging assembly images the sample chamber.

35. The system of claim 34, wherein the imaging assembly has an imaging plane, the sample chamber moving along the imaging plane as the reaction valve is rotated.

36. The system of claim 34, wherein the at least one image includes first, second, and third images and the imaging assembly is configured to sequentially obtain the first, second, and third images, the positioning assembly is configured to rotate the reaction valve after the first image is obtained to position the sample chamber for the second image and is also configured to rotate the reaction valve after the second image is obtained to position the sample chamber for the third image.

37. The system of claim 36, wherein the positioning assembly is configured to rotate the sample chamber an arcuate distance after each of the first and second images is obtained.

38. The system of claim 34, further comprising a manifold having first and second manifold ports, wherein the positioning assembly is configured to rotate the reaction valve from a first rotational position to a second rotational position, the reaction valve being in fluid communication with the first manifold port when the reaction valve is in the first rotational position and being in fluid communication with the second manifold port when the reaction valve is in the second rotational position.

39. The system of claim 38, wherein the at least one image includes first and second images, the reaction valve being in the first rotational position when the first image is obtained and in a different rotational position when the second image is obtained.

40. The system of claim 34, wherein the imaging assembly includes an objective lens positioned proximate to the sample chamber for imaging the sample chamber.

41. The system of claim 34, wherein the imaging assembly includes first and second Objective lenses positioned proximate to the sample chamber for imaging the sample chamber, the objective lenses imaging different portions of the sample chamber.

42. The system of claim 34, wherein the at least one image includes a wide-field image.

43. A method of imaging a sample in a reaction valve, the method comprising:
providing a reaction valve having a sample chamber and an imaging assembly configured to image the sample chamber;
rotating the reaction valve about an axis of rotation to a first rotational position, the sample chamber moving relative to the imaging assembly;
imaging the sample chamber; and
rotating the reaction valve about the axis of rotation to a second rotational position.

44. The method of claim 43, wherein the imaging assembly has an imaging plane, the sample chamber moving along the imaging plane as the reaction valve is rotated.

45. The method of claim 43, wherein the imaging assembly comprising an objective lens for imaging the sample chamber, wherein the objective lens is not moved in a lateral direction along the imaging plane.

46. The method of claim 43, wherein the imaging operation includes imaging multiple portions of the sample chamber concurrently.

47-56. (canceled)

57. A method of at least one of generating or analyzing a biological or chemical sample using a reaction valve that includes a sample chamber, the reaction valve being positioned adjacent to a manifold along an interface between the reaction valve and the manifold, the reaction valve having a valve inlet and the manifold having a plurality of ports, the valve inlet and the plurality of ports opening to the interface, the method comprising:
(a) flowing, through a first port of the manifold, a first fluid into the sample chamber, the first fluid including a reaction component;
(b) moving the manifold and the reaction valve relative to each other along the interface to fluidly disconnect the valve inlet from the first port and to fluidly connect the valve inlet with the second port;
(c) flowing a second fluid through the second port and into the sample chamber; and
(d) imaging a portion of the sample chamber.

58. The method of claim 57, further comprising repeating (a)-(d) a plurality of times.

59. The method of claim 57, wherein the reaction component includes at least one labeled nucleotide configured to react with the sample.

60. The method of claim 59, further comprising flowing a third fluid into the sample chamber after (d) to remove a component of the sample.

61. The method of claim 60, wherein flowing a third fluid into the sample chamber includes cleaving the component of the sample, the component being a reversible terminator.

62. The method of claim 58, wherein (d) includes:
imaging a first portion of the sample chamber prior to or during step (b); and imaging a second portion of the sample chamber during or after step (b).

63. The method of claim 58, wherein the sample includes nucleic acids and the method comprises performing sequencing-by-synthesis (SBS) to obtain sequencing information about the sample.
